SEQUENCE LISTING (6 sequences)

SEQ ID NO: 1 - DNA, 1515 bp, Homo sapiens
gatcgtgtaggccggccgcaccatggggggcagcccagcccagccgcggtaaacgccgacctccgccgccgcccgcgccgcgtctgccccctccgctgcggctctctggacgccatcccctcctcacctcgaagccaacatgaaggagacccggggctacggaggggatgcccccttctgcacccgcctcaaccactcctacacaggcatgtgggcgcccgacggttccgccgaggcgcggggcaacctcacgcgccctccagggtctggcgaggattgcggatcggtgtccgtggccttcccgatcaccatgctgctcactggtttcgtgggcaacgcactggccatgctgcttgtgtcgcgcagctaccggcgccgggagagcaagcgcaagaagtccttcctgctgtgcatcggctggctggcgctcaccgacctggtcgggcagcttctcaccaccccggtcgtcatcgtcgtgtacctgtccaagcagcgttgggagcacatcgacccgtcggggcggctctgcacctttttcgggctgaccatgactgttttcgggctctcctcgttgttcatcgccagcgccatggcggtcgagcgggcgctggccatcagggcgccgcactggtatgcgagccacatgaagacgcgtgccacccgcgctgtgctgctcggcgtgtggctggccgtgctcgccttcgccctgctgccggtgctgggcgtgggccagtacaccgtccagtggcccgggacgtggtgcttcatcagcaccgggcgagggggcaacgggactagctcttcgcataactggggcaaccttttcttcgcctctgcctttgccttcctggggctcttggcgctgacagtcaccttttcctgcaacctggccaccattaaggccctggtgtcccgctgccgggccaaggccacggcatctcagtccagtgcccagtggggccgcatcacgaccgagacggccattcagcttatggggatcatgtgcgtgctgtcggtctgctggtctccgctcctgataatgatgttgaaaatgatcttcaatcagacatcagttgagcactgcaagacacacacggagaagcagaaagaatgcaacttcttcttaatagctgttcgcctggcttcactgaaccagatcttggatccttgggtttacctgctgttaagaaagatccttcttcgaaagttttgccagatcaggtaccacacaaacaactatgcatccagctccacctccttaccctgccagtgttcctcaaccttgatgtggagcgaccatttggaaagataatgaaagaacggagttggacattttattgcaattcctgcttccctgaatttgcatatttcttcccacctgagaaggataattatatattttaatttggattatttcttcattttatcttttatttaatgattgttttgtcagtaatacccatggagatcaaatttattattataatccatgcctctgaatattagattggtttc

SEQ ID NO: 2 - protein, 390 aa, Homo sapiens [sequence omitted]

SEQ ID NO: 3 - DNA, 459 bp, Homo sapiens
atgcctgcccacagcctggtgatgagcagcccggccctcccggccttcctgctctgcagcacgctgctggtcatcaagatgtacgtggtggccatcatcacgggccaagtgaggctgcggaagaaggcctttgccaaccccgaggatgccctgagacacggaggcccccagtattgcaggagtgaccccgacgtggaacgctgcctcagggcccaccggaacgacatggagaccatctaccccttccttttcctgggcttcgtctactcctttctgggtcctaacccttttgtcgcctggatgcacttcctggtcttcctcgtgggccgtgtggcacacaccgtggcctacctggggaagctgcgggcacccatccgctccgtgacctacaccctggcccagctcccctgcgcctccatggctctgcagatcctctgggaagcggcccgccacctgtga

SEQ ID NO: 4 - protein, 152 aa, Homo sapiens
MPAHSLVMSSPALPAFLLCSTLLVIKMYVVAIITGQVRLRKKAFANPEDALRHGGPQYCRSDPDVERCLRAHRNDMETIYPFLFLGFVYSFLGPNPFVAWMHFLVFLVGRVAHTVAYLGKLRAPIRSVTYTLAQLPCASMALQILWEAARHL

SEQ ID NO: 5 - DNA, 4465 bp, Homo sapiens
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 6 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

(19) United States
(12) Patent Application Publication
(10) Pub. No.: US 2011/0008258 A1
(43) Pub. Date: Jan. 13, 2011

(54) METHODS RELATING TO BREATHING DISORDERS

(75) Inventors: Eric Herlenius, Stockholm (SE); Per-Johan Jakobsson, Stockholm (SE); Annika Hofstetter Olsson, New York, NY (US)

Correspondence Address: BIRCH STEWART KOLASCH & BIRCH, PO BOX 747, FALLS CHURCH, VA, 22040-0747, US

(73) Assignee: SAMSARA MEDICIN AB, Stockholm (SE)

(21) Appl. No.: 12/742,342

(22) PCT Filed: Nov. 12, 2008
(86) PCT No.: PCT/GB2008/003856

§ 371 Date: Sep. 14, 2010

Related U.S. Application Data
(60) Provisional application No. 60/987,217, filed on Nov. 12, 2007.

(51) Int. Cl. A61K 31/7088; A61K 31/165; A61K 31/404; A61K 31/42; A61K 31/415; A61K 31/34; G01N 33/88; G01N 33/68; A61K 49/00; A61P 11/00

(52) U.S. Cl. 424/9.2; 514/44 R; 514/618; 514/418; 514/378; 514/406; 514/473; 436/129; 436/86; 436/93

(57) ABSTRACT

Methods for treating breathing disorders by inhibition of the induced PGE₂ pathway in a mammalian subject, methods for assessing apnea, hypoxic ischemic encephalopathy or perinatal asphyxia by detecting an elevated level of PGE₂, or a metabolite thereof, in a sample from the subject compared with a control level, and in vitro and in vivo screening methods for medicaments for treating breathing disorders are disclosed.

### FIELD OF THE INVENTION

The present invention relates to methods for treating breathing disorders, such as apnea, to diagnostic and screening methods and compositions for use in such methods.

### BACKGROUND TO THE INVENTION

Apnea and Sudden Infant Death Syndrome (SIDS) represent major medical concerns in the neonatal population, and infection may play a crucial role in their pathogenesis. Apnea is a common presenting sign of infection in neonates, and mild viral or bacterial infection precedes death in the majority of SIDS victims (1, 2, 111).

Children with non-optimal or delayed brainstem respiratory control such as preterm infants (all during their first year of life and several also beyond early childhood), children with Congenital Central Hypoventilation Syndrome (CCHS) (79), Rett's Syndrome and Prader Willi Syndrome (PWS) (80) have periodic irregular breathing with apnea that are increased during sleep as well as during infectious episodes when the resulting apnea can be, and sometimes is, fatal if external- or auto-resuscitation does not occur.

In children that die in SIDS mild infection often precedes death and emerging evidence indicates that brainstem dysfunction and failure to auto resuscitate from hypoxic events are associated with the majority of these unexplained deaths (81, 82).

In older children and adults there is an increased risk for potentially fatal respiratory dysfunction in children and adults with acquired or congenital impaired respiratory control e.g., Rett's Syndrome and PWS, but also children and adults with sleep apnea syndrome and adults with Parkinson's disease have an impaired respiratory control and often die in association with an infection (83). Respiratory disorders (respiratory insufficiency or infections) have been identified as the most common cause of death among PWS children (107). Moreover, snoring and obstructive sleep apnea syndrome (OSAS) in children may lead to disturbed sleep and impaired neurocognitive development, resulting in long-term dysfunction. This is worsened by respiratory infection and prevalence of additive risk factors such as smoking in the environment and asthma (108-111).

Potentially deleterious and life threatening breathing disorders are common also in the adult population. Hence, an impaired ventilatory response to hypoxia may play a critical role in Parkinson's disease, sleep-related breathing disorders such as sleep-apneic syndrome and OSAS in adults.

Pro-inflammatory cytokines such as interleukin-1β (IL-1β) may serve as key mediators between these events (3). IL-1β is produced during an acute phase immune response to infection and inflammation and evokes a variety of sickness behaviours (for review, see (4)). Previous studies indicate that this immunomodulator also alters respiration and autoresuscitation (5-10). IL-1β induces expression of the immediate-early gene c-fos in respiration-related regions of the brainstem such as the nucleus tractus solitarius (NTS) and rostral ventrolateral medulla (RVLM) (11). However, IL-1β is a large lipophobic protein that does not readily diffuse across the blood-brain barrier. Furthermore, the NTS and RVLM do not appear to express IL-1 receptor mRNA (12), and IL-1β does not alter brainstem respiration-related neuronal activity in vitro (5).

We previously showed that indomethacin, a non-specific COX inhibitor, attenuates the respiratory depression induced by IL-1β (5). PGE₂ itself depresses breathing in fetal and newborn sheep in vivo (17-19) and inhibits respiration-related neurons in vitro (5). Neonatal urinary prostanoid excretion has been investigated in preterm and term infants (112) and a relationship identified between PGE-M and apnea in preterm infants (113).

Indomethacin has been used previously to treat apnea of prematurity (45). However, indomethacin causes multiple adverse effects in the newborn population (46). Adverse effects associated with indomethacin use in neonates may include drug-induced reductions in renal, intestinal, and cerebral blood flow (46). Caffeine is used in the treatment of respiratory dysfunction as are continuous positive airway pressure (CPAP) and supplemental oxygen. Furthermore, acute treatment with naloxone (an opioid receptor antagonist) has also been used. However, there is a clear need for treatment modalities of breathing disorders, particularly for treatment of apnea.

### DISCLOSURE OF THE INVENTION

The present inventors have now discovered that the induced PGE₂ pathway is a key regulator of the respiratory response to infection and hypoxia (see also 114). The induced PGE₂ pathway is depicted in FIG. 6 herein.

IL-1β binds to IL-1 receptors on vascular endothelial cells of the blood-brain barrier and induces cyclooxygenase-2 (COX-2) and microsomal prostaglandin E synthase-1 (mPGES-1) activity (for review, see (13)). COX-2 catalyzes the formation of prostaglandin H₂ (PGH₂) from arachidonic acid, and mPGES-1 subsequently catalyzes the synthesis of prostaglandin E₂ (PGE₂) from PGH₂. PGE₂ is then released into the brain parenchyma where it recently has been shown to mediate several central effects of IL-1β, e.g., fever induction (14), behavioural responses (15), and neuroendocrine changes (16). As described further herein, prostaglandin also mediates the ventilatory effects of IL-1β (54). Furthermore, E-prostanoid receptor subtype 3 (EP3R) receptors for PGE₂ are located in respiration-related regions of the brainstem: the NTS and RVLM (20, 21).

As described herein, IL-1β adversely affects central respiration via mPGES-1 activation and PGE₂ binding to brainstem EP3R, resulting in increased apnea frequency and failure to autoresuscitate after a hypoxic event. Breathing disorders associated with the induced PGE₂ pathway may, therefore, be ameliorated by targeting this pathway at one or more sites, such as by inhibiting COX-2, inhibiting mPGES-1 and/or inhibiting EP3R.

Accordingly, in one aspect the present invention provides a method of treating a breathing disorder in a mammalian subject, comprising administering to a subject in need of treatment a therapeutically effective amount of a composition comprising: an inhibitor of E-prostanoid receptor subtype 3 (EP3R); an inhibitor of microsomal prostaglandin E synthase-1 (mPGES-1); and/or a selective inhibitor of cyclooxygenase-2 (COX-2).

The ability to block the precise pathway involved in the induction of breathing disorders, such as apnea, using a composition that targets one or more steps in the inducible PGE₂ pathway described herein is expected to minimise the deleterious effects associated with less selective therapies. For example, by targeting COX-2 selectively, mPGES-1 and/or EP3R, a breathing disorder as described further herein may be ameliorated while minimising adverse side effects, such as those associated with use of the non-selective COX inhibitor indomethacin.

In a further aspect the present invention provides a composition for use in a method of treating a breathing disorder in a mammalian subject, wherein the composition comprises: an inhibitor of EP3R; an inhibitor of mPGES-1; and/or a selective inhibitor of COX-2.

In a further aspect the present invention provides use of a composition in the manufacture of a medicament for treating a breathing disorder in a mammalian subject, wherein the composition comprises: an inhibitor of EP3R; an inhibitor of mPGES-1; and/or a selective inhibitor of COX-2.

In a further aspect the present invention provides a method of assessing susceptibility to, or presence of, a breathing disorder in a mammalian subject, comprising
detecting the level of prostaglandin-E₂ (PGE₂), or a metabolite thereof, in a sample from the mammal, and
comparing the level in the sample with a control level of PGE₂, or the metabolite thereof,
wherein an elevated level of PGE₂, or the metabolite thereof, in the sample compared with the control level of PGE₂, or the metabolite thereof, indicates susceptibility to, or presence of, a breathing disorder in the subject.

The present inventors provide evidence herein for the central role of PGE₂ in breathing disorders such as apnea and diminished autoresuscitation following hypoxia. In particular, increased levels of PGE₂ and/or metabolites thereof in cerebrospinal fluid (CSF) and/or in urine are associated with increased apnea frequency and decreased ability to autoresuscitate following hypoxia. A correlation between C-reactive protein (CRP) levels, PGE₂ levels and apnea, indicates that monitoring PGE₂ levels and/or metabolites thereof alone or in conjunction with markers of infection, such as CRP, can provide diagnostic benefits in relation to breathing disorders and susceptibility thereto. The rapid synthesis of PGE₂ in response to cytokine and hypoxic stimulation make it particularly useful in the diagnosis and surveillance of breathing disorders in mammals, such as of increased apneas in infants, due to suspected infection or asphyxia.

The present inventors have surprisingly found that levels of urinary prostaglandin metabolites (u-PGEM) are elevated in infants with ongoing infection and associated apnea, children with PWS and a sub-population of adults having sleep apnea (including those having a high apnea index). The ability to derive a measure of PGE₂ levels using a specific and sensitive assay on urine provides a non-invasive method for prediction and assessment of breathing disorders (particularly apnea) that may be applied to a surprisingly large range of patient age groups. Among infants having an infection and associated apnea, the elevation of u-PGEM levels appears to occur at an earlier stage than elevation of CRP levels. Thus, assessment of levels of PGE₂ and/or metabolites thereof in a biological sample (e.g. urine, blood or CSF) offers advantages for diagnosis, treatment and management of patients having infection-associated inflammation and breathing dysfunction in comparison with assessment of levels of CRP.

Accordingly, the present invention provides a method of assessing the presence of and/or severity of apnea in a human subject, comprising
detecting the level of one or more PGE₂ metabolites in a urine sample obtained from the subject, and
comparing the level in the sample with a control level of said one or more PGE₂ metabolites,
wherein a level of said one or more PGE₂ metabolites that is at least 20%, at least 50%, at least 100% or at least 200% greater in the sample compared with the control level of said one or more PGE₂ metabolites indicates the presence of and/or greater severity of apnea in the subject. In certain cases the human subject has obstructive sleep apnea syndrome (OSAS), Prader-Willi Syndrome, Congenital Hypoventilation Syndrome and/or Rett's Syndrome. In certain cases the human subject is greater than 16 years of age; between 1 and 16 years of age; or between 0 and 1 year of age.

The present inventors describe herein the elevation of PGE₂ in subjects following birth asphyxia and the correlation of PGE₂ with hypoxic ischemic encephalopathy (HIE). These results show that PGE₂ and metabolites thereof provide a powerful prognostic marker for neurological damage caused by a deficit in perinatal cerebral oxygen delivery. Moreover, the results indicate that the degree of hypoxia a subject has been exposed to is reflected in levels of PGE₂ and metabolites thereof detected in a sample (e.g. a CSF, urine or blood sample).

Accordingly, in a further aspect the present invention provides a method of assessing susceptibility to, or presence of, hypoxic ischemic encephalopathy (HIE) in a mammalian subject, comprising
detecting the level of prostaglandin-E₂ (PGE₂), or a metabolite thereof, in a sample from the subject, and
comparing the level in the sample with a control level of PGE₂, or the metabolite thereof,
wherein an elevated level of PGE₂, or the metabolite thereof, in the sample compared with the control level of PGE₂ indicates susceptibility to, or presence of, HIE in the subject.

In a further aspect the present invention provides a method of assessing hypoxia or severe hypoxia-asphyxia (such as perinatal asphyxia) to which a mammalian subject has been subjected, comprising
detecting the level of prostaglandin-E₂ (PGE₂), or a metabolite thereof, in a sample from the subject, and
comparing the level in the sample with a control level of PGE₂, or the metabolite thereof,
wherein an elevated level of PGE₂, or the metabolite thereof, in the sample compared with the control level of PGE₂ indicates that the subject has been subjected to hypoxia or hypoxia-asphyxia (such as perinatal asphyxia).

In a further aspect the present invention provides a method for identifying a substance for use in treating a breathing disorder in a mammal, comprising assaying a test substance for the ability to inhibit the induced PGE₂ pathway, for example assaying a test substance for the ability to inhibit one or more of the following:
(a) COX-2-mediated synthesis of PGH₂;
(b) mPGES-1-mediated conversion of a cyclic endoperoxide substrate of mPGES-1 into a product which is the 9-keto, 11α hydroxy form of the substrate; and
(c) EP3R agonist-mediated activation of EP3R,
wherein inhibition of the induced PGE₂ pathway, for example inhibition of one or more of (a), (b) and (c), indicates that the test substance is a substance for use in treating a breathing disorder in a mammal.

A test substance found to have the ability to inhibit the induced PGE₂ pathway may be formulated into a composition comprising one or more further components, such as a pharmaceutically acceptable excipient. Such a composition may be used in a method of treating a breathing disorder in a mammal.

The realization of the central importance of the induced PGE₂ pathway and its contribution to breathing disorders such as apnea (see FIG. 6), provides the basis for identifying agents that may have therapeutic utility in the treatment of breathing disorders. In particular, a method of screening a test substance for the ability to inhibit one or more of the following:
(a) COX-2-mediated synthesis of PGH₂;
(b) mPGES-1-mediated conversion of a cyclic endoperoxide substrate of mPGES-1 into a product which is the 9-keto, 11α hydroxy form of the substrate; and
(c) EP3R agonist-mediated activation of EP3R,
   may be carried out using one or more in vitro assays. Screening test substances for inhibitory activity may be scaled-up more readily than a screening method that relies on measuring effects of a test substance on an animal model of a breathing disorder. This may be advantageous where an initial in vitro screen is carried out prior to screening test substances in an animal model of a breathing disorder. In this way, promising substances with suitable in vitro pharmacological activity may be selected for further investigation in vivo.

In a further aspect the present invention provides a method for identifying a substance for use in treating a breathing disorder in a mammal, comprising:
administering a test substance to a test mammal, wherein the test substance is an inhibitor of the induced PGE₂ pathway, for example an inhibitor of EP3R, an inhibitor of mPGES-1 and/or a selective inhibitor of COX-2; and
determining the severity of a sign or symptom of a breathing disorder in the test mammal compared to the sign or symptom in a control mammal to which the test substance has not been administered,
wherein a lower severity of the sign or symptom of the breathing disorder in the test mammal than in the control mammal indicates that the test substance is a substance for use in treating a breathing disorder in a mammal.

The method of this aspect of the invention may further comprise an earlier stage, which stage comprises determining whether a test substance has the ability to inhibit the induced PGE₂ pathway, such as the ability to act as an inhibitor of EP3R, an inhibitor of mPGES-1 and/or a selective inhibitor of COX-2.

A test compound found to have the ability to lower the severity of a sign or symptom of a breathing disorder and thereby treat a breathing disorder may be formulated into a composition comprising one or more further components, such as a pharmaceutically acceptable excipient. Such a composition may be used in a method of treating a breathing disorder in a mammal.

In a further aspect the present invention provides a method of inducing respiratory depression in a mammal, comprising administering to the mammal an effective amount of a composition comprising: an E-prostanoid receptor subtype 3 (EP3R) agonist that is other than PGE₂, a microsomal prostaglandin E synthase-1 (mPGES-1) activator and/or a selective cyclooxygenase-2 (COX-2) activator.

Induction of respiratory depression in a mammal may have particular utility in the study of breathing disorders. For example, induction of respiratory depression in a mammal may be useful in the provision of an animal model of breathing disorders such as apnea, hypoxia and/or diminished autoresuscitation. Such models may be useful in testing whether EP3R or mPGES-1 activation occurs in animal models for apnea, such as sleep apnea, and Parkinson's disease, such as respiratory dysfunction associated with Parkinson's disease.

PGE₂, released during hypoxia, may have acute neuroprotective effects, for example, through stimulating EP3R-Gᵢ-activation and subsequent lowering of cAMP and reduction of neuronal activity leading to increased brain resistance to acute hypoxia.

The present invention includes the combination of the aspects and preferred features described except where such a combination is clearly impermissible or is stated to be expressly avoided. These and further aspects and embodiments of the invention are described in further detail below and with reference to the accompanying examples and figures.

### DESCRIPTION OF THE FIGURES

FIG. 1 shows IL-1β and anoxia rapidly inducing brainstem mPGES-1. mPGES-1 activity in the microsomal fraction of cortex and brainstem, including endothelial cells of the blood-brain barrier (BBB), was analyzed in 9 d-old mice (n=33) treated with IL-1β or vehicle and subjected to normoxia or normoxia plus anoxia (100% N₂, 5 min). A) In wildtype mice, mPGES-1 activity was measured at 90 min after NaCl (Control) or 90 min and 180 min after IL-1β treatment. Higher endogenous mPGES-1 activity was observed in the brainstem compared to cortex in control mPGES-1^{+/+} mice. In addition, IL-1β induced mPGES-1 activity in a time-dependent manner. B) At 90 min, IL-1β-treated mice exhibited approximately two-fold higher activity in the brainstem compared to saline-treated mice. Anoxia also significantly induced mPGES-1 activity. Moreover, the effects of IL-1β and transient anoxic exposure were additive. When IL-1β-treated mice were exposed to anoxia, four-times higher activity was observed in the brainstem compared to control mice. However, mice with genetic deletion of mPGES-1 gene displayed negligible activity in response to IL-1β and anoxia. Data are presented as mean±SEM. ** P<0.01; *** P<0.001.

FIG. 2 shows IL-1β depression of respiration via mPGES-1 activation. Using whole-body flow plethysmography, basal respiration and the ventilatory response to hyperoxia were examined in 9 d-old mPGES-1 WT mice (n=66) and mPGES-1 KO mice (n=34) following i.p. administration of either IL-1β (n=52) or NaCl (n=48). A) Plethysmograph recordings illustrate breathing during normoxia and hyperoxia in wildtype mice given NaCl or IL-1β (5 s period, breath amplitude 1 μl/s). B, C) All mice responded to hyperoxia with a reduction in respiratory frequency (f_{R}, breaths/min). IL-1β depressed f_{R} to a greater extent than NaCl in mPGES-1^{+/+} mice, whereas IL-1β did not alter respiration during normoxia or hyperoxia in mPGES-1^{-/-} mice. mPGES-1^{+/+} mice exhibited a greater respiratory depression during hyperoxia compared to mPGES-1^{-/-} mice. Data are presented as mean±SEM. * P<0.05 compared to mPGES-1^{+/+} mice given NaCl.

FIG. 3 shows IL-1β reduction of anoxic survival via mPGES-1. 9 d-old mPGES-1^{+/+} mice (n=37) and mPGES-1^{-/-} mice (n=20) were exposed to 5 min anoxia (100% N₂) at 80 min after peripheral administration of IL-1β (n=29) or vehicle (n=28). A) Plethysmograph recording of mPGES-1^{+/+} mouse given NaCl depicting the initial hyperpnea and subsequent gasping response to anoxia. The mouse autoresuscitated after 100% O₂ was administered. B) Plethysmograph recording of mPGES-1^{+/+} mouse given IL-1β showing the brief hyperpnea period and subsequent gasping response to anoxia. The mouse failed to autoresuscitate after 100% O₂ was administered. The number of gasps (C) tended to differ between groups (Wilcoxon X², P=0.06). When comparing treatment effects within each genotype, IL-1β decreased the number of gasps in wildtype mice, whereas this effect was not observed in mice lacking mPGES-1. D) IL-1β reduced the survival rate anoxic compared to NaCl in mPGES-1^{+/+} mice, but not in mPGES-1^{-/-} mice. Data are presented as mean±SEM. * P<0.05; ** P<0.01.

FIG. 4 shows PGE₂ depression of brainstem respiratory activity and induction of apnea via brainstem EP3 receptors. Respiration was examined in neonatal mice with EP3R^{+/+} (n=13) and EP3R^{-/-} (n=25) genotypes following administration of PGE₂ (n=19) or NaCl (n=19). A) PGE₂ was injected (icy) at 0 min followed by normoxia and a 1 min hyperoxic challenge in newborn EP3R^{+/+} (▪) and EP3R^{-/-} (□) mice. The EP3R^{+/+} mouse exhibited a lower respiratory frequency (f_{R}, breaths/min) and an irregular respiratory rhythm with elevated coefficient of variation (C.V.) during normoxia and hyperoxia due to apneic breathing. In the EP3R^{-/-} mouse, basal f_{R} did not decrease following the post-anesthesia period, and there was less variability in the respiratory pattern. No temperature difference or dependency was observed during the first 20 min after icy administration of PGE₂. B) Plethysmograph recordings (10 s periods with breath amplitude of 1 μl/s) demonstrate apnea episodes in response to PGE₂ during normoxia in an EP3R^{+/+} mouse, but not in an EP3R^{-/-} mouse. C) In EP3R^{+/+} mice, PGE₂ induced more apneas during normoxia and hyperoxia compared to vehicle. This effect of PGE₂ was not observed in EP3R^{-/-} mice. D) In “en bloc” brainstem spinal-cord preparations from 2-3 d old EP3R^{+/+} pups (▪, n=5), PGE₂ (20 μg/l) reversibly depressed respiratory rhythm generation to 64±5% of control frequency (f_{R}) (ANOVA repeated measures design, P<0.01). PGE₂ did not affect respiratory activity in preparations from EP3R^{-/-} mice (□, n=6). E) In transverse medullary sections, respiration-related neurons within the rostral ventrolateral medulla (RVLM) ventral to the nucleus ambiguus (NA) and including the preBötzinger complex co-express NK1R and EP3R. Both NK1R and EP3R expression are exhibited. The arrows indicate EP3R and NK1R co-localization in some RVLM respiration-related neurons. F) NK1R, but no EP3R, expression was identified in an EP3R^{-/-} mouse. Scale bar=100 μm. Data are presented as mean±SEM. * P<0.05 compared to EP3R^{+/+} mice given NaCl.

FIG. 5 shows correlation of PGE₂ in cerebrospinal fluid with apnea index in neonates. Cerebrospinal fluid (CSF) was collected from infants in the neonatal intensive care unit who had clinical indications for lumbar puncture (n=12, mean postnatal age 16±4 d, mean gestational age 32±2 week). Infants then underwent a cardiorespiratory recording (duration 9.2±2.4 h). PGE₂ concentrations in the CSF were analyzed using a standardized enzyme immunoassay (EIA) protocol and correlated to the infectious marker C-reactive protein (CRP) and apnea index (# apneas/h). Central PGE₂ concentrations were positively correlated to the CRP levels in blood (P=0.01). Moreover, a striking association was observed between central PGE₂ concentrations and apnea index (P<0.05). Here, we distinguish between undetectable levels of PGE₂ (0±0 pg/ml) compared to high levels of PGE₂ (52±22 pg/ml). Data are presented as mean±SEM.

FIG. 6 depicts a model for IL-1β-induced respiratory depression and autoresuscitation failure via a prostaglandin E₂-mediated pathway. During a systemic immune response, the pro-inflammatory cytokine interleukin-1β (IL-1β) is released into the peripheral blood stream. It binds to its receptor (IL-1R) located on endothelial cells of the blood-brain barrier (BBB). Activation of IL-1R induces the synthesis of prostaglandin H₂ (PGH₂) from arachidonic acid (AA) via cyclooxygenase-2 (COX-2) and the synthesis of prostaglandin E₂ (PGE₂) from PGH₂ via the rate limiting enzyme microsomal prostaglandin E synthase-1 (mPGES-1). PGE₂ is released into the brain parenchyma and binds to its EP3 receptor (EP3R) located in respiratory control regions of the brainstem, e.g., nucleus of the solitary tract (NTS) and the rostral ventrolateral medulla (RVLM). This results in depression of central respiration-related neurons and breathing, which may fatally decrease the ability to gasp and autoresuscitate during hypoxic events.

FIG. 7 A) Correlation of PGE₂-metabolite concentration in CSF with the degree of asphyxia and adverse outcome in human infants. The PGE₂-metabolite in CSF was obtained during lumbar puncture taken <24 hours after birth and correlates to Hypoxic Ischemic Encephalopathy (HIE). B) Correlation of PGE₂-metabolite concentration in CSF with the APGAR score at 5 minutes after birth of human infants.

FIG. 8 shows urinary prostaglandin metabolite (u-PGEM) levels for healthy control adults vs. adults with obstructive sleep apnea syndrome. Measurements made by triple quadropole mass spectrometry-tetranor PGEM method (PGE metabolites expressed as pmol PGEM/μg creatinine). The apnea group displays a far greater diversity of values compared with the controls, including a sub-group with much higher levels of PGEM (dotted ellipse).

FIG. 9 shows urinary prostaglandin (u-PGEM) levels for healthy control children vs. children having Prader-Willi Syndrome (PWS) (3-16 years of age). Measurements made by triple quadropole mass spectrometry-tetranor PGEM method (PGE metabolites expressed as pmol PGEM/μg creatinine). The PWS group exhibits significantly elevated u-PGEM levels compared with the controls.

FIG. 10 shows urinary prostaglandin (u-PGEM) levels for healthy control infants (1 month-1 year of age) vs. infants with ongoing inflammation, virus bronchiolitis and associated apnea. Measurements made by triple quadropole mass spectrometry-tetranor PGEM method (PGE metabolites expressed as pmol PGEM/μg creatinine). The apnea and inflammation group exhibits significantly elevated u-PGEM levels compared with the controls.

### DETAILED DESCRIPTION OF THE INVENTION

### Breathing Disorder

The invention contemplates a range of breathing disorders that involve aberrant central control of respiration and/or ventilation. In particular, the breathing disorder may involve abnormal—such as irregular or decreased—breathing frequency, fewer and/or shorter gasps, decreased tidal volume and/or impaired breathing response to hypoxia. The breathing disorder may be periodic breathing.

### Apnea

The breathing disorder may be apnea. Apnea means a cessation of breathing, which may be temporary or permanent. Apnea may be determined by, for example, impedance pneumography and recorded via an event monitoring system, as described further herein. Apnea frequency may be defined as the number of events exceeding a pre-determined apnea threshold. Definitions are known to vary depending on the age of the subject under consideration. In some embodiments, such as when the mammal is a human infant of less than five years of age, apnea may be defined as a ≧10 sec reduction of the mean impedance signal amplitude during the preceding 0.5 s to less than 16% of the mean amplitude measured during the preceding 25 s. In other embodiments, such as when the mammal is a human adult, apnea may be defined as >10 sec pause in breathing. In certain embodiments, apnea may be defined as a respiratory pause exceeding two respiratory cycles.

Sleep-Related Breathing Disorder

The breathing disorder may be a disorder that occurs during sleep. Sleep apnea in infants may, in severe cases, be associated with increased risk of sudden infant death syndrome (SIDS). Also contemplated herein is adult sleep apnea, which may include snoring.

### Periodic Breathing

Sleep disordered breathing is characterized by periodic breathing, episodes of hypoxia and repeated arousals from sleep; symptoms include excessive daytime sleepiness, impairment of memory, learning and attention. Both intermittent hypoxia and sleep fragmentation can independently lead to neuronal defects in the hippocampus and pre frontal cortex; areas closely associated with neural processing of memory and executive function.

Periodic breathing, or alternating periods of hyperpnea and apnea, is a common breathing pattern in premature infants. Clinically important apnea of prematurity is almost always associated with periodic breathing. The periods of hypopnea may decrease PaO₂, this in young children or patients with previously affected brainstem respiratory centres, may decrease breathing. This occurs via a hypoxic induced depression of brainstem respiratory centres mediated partly by adenosine and PGE2 release (54, 85). The periods of hyperpnea or hyperventilation may decrease PaCO₂ and reduce the stimulus to breathe, resulting in apnea.

The late preterm infant continues to have a slightly blunted ventilatory response to CO₂, spends more than 50% of sleep time in REM, and continues to have apnea and periodic breathing, with a prevalence of 10% compared with 60% in infants born at less than 1500 g.

True periodic breathing or apnea emerges when the segments of the cycle with the lowest depth of breathing actually become pauses—apnea.

In neonates, children and adults sleep disordered periodic breathing and intermittent hypoxia is associated with neural deficit, and such lesions may lead to cognitive dysfunction (92, 93).

### Failure to Autoresuscitate

The breathing disorder may be failure to autoresuscitate following a hypoxic event. Autorescusciation is the brain's ability to arouse itself from sleep or severe hypoxic depression of breathing movements with a forceful regular inspirational gasping during prolonged hypoxia. This enables the body and blood saturation to regain its oxygenation.

Mammals typically exhibit a biphasic response to anoxia with an initial increase in ventilation (i.e. hypernea) followed by a hypoxic ventilatory depression (i.e. primary apnea, gasping, secondary apnea). Administration of oxygen following hypoxia then leads to autoresuscitation. Failure to autoresuscitate following hypoxia may lead to death without intervention.

### SIDS

The breathing disorder may be a disorder that results in sudden infant death syndrome (SIDS). SIDS (also known as “cot death”) is the sudden unexpected death of an infant, generally under two years old. The cessation of breathing and failure to autoresuscitate, which may occur during sleep, may lead to death described as SIDS. Thus, a breathing disorder of particular severity may lead to a sudden unexpected death. In certain embodiments, the present invention specifically contemplates breathing disorders of a severity sufficient to result in a sudden unexpected death.

### Infection-Related Breathing Disorder

The breathing disorder may be associated with viral and/or bacterial infection. Various infection-related markers may be increased during infection, such as CRP, white blood cell count and proinflammatory cytokines, including IL-1β, which may indicate that the breathing disorder has an infection-related component.

In certain embodiments of the invention the breathing disorder may be an IL-1β-related breathing disorder. IL-1β is produced during an acute phase immune response to infection and inflammation. As disclosed herein, IL-1β acts on IL-1 receptors on vascular endothelial cells of the blood brain barrier and induces COX-2, leading to stimulation of the induced PGE₂ pathway and ultimately central respiratory depression resulting in increased apnea frequency and failure to autoresuscitate after a hypoxic event. Elevated blood levels of IL-1β compared with a control level of IL-1β, may indicate that the breathing disorder is an IL-1β-related breathing disorder.

In certain embodiments the mammal or mammalian subject may be a human suffering from acquired or congenital impaired respiratory control, including an autonomic dysfunction disorder, e.g. Prader Willi Syndrome (PWS), congenital hypoventilation syndrome (“CCHS”, also known as “Ondine's curse”) and/or Rett's Syndrome. Infants having PWS, CCHS or Rett's Syndrome are at increased risk of death due to respiratory dysfunction during infectious events.

### Hypoxic Ischemic Encephalopathy

Hypoxic ischemic encephalopathy (HIE) is the term used to designate the condition of a full term infant who has experienced a perinatal deficit in cerebral oxygen delivery leading to disruption of cerebral energy metabolism (97). This condition can lead to death or severe neurological sequelae.

Studies of the cerebral energy metabolism with magnetic resonance spectroscopy have lead to the hypothesis that after a primary disruption of oxygen delivery to the brain cells there occurs a secondary phase of neuronal loss that can be delayed for hours or days (98, 99), which has also been shown in animal studies (100). This delay in neuronal damage is believed to be due in part to the release of inflammatory mediators into the immediate environment in response to the injury.

Interactions between the nervous and immune systems are important in many aspects of disease. Neither the pathophysiology nor the etiology of HIE is fully understood. Recently other causes than hypoxia-ischemia have been emphasized, such as intrauterine or neonatal inflammation (101, 102) and attention has turned to cytokines as mediators of the injury (103). There is also evidence supporting the involvement of inflammatory cascade in the pathogenesis of ischemic brain injury (104). Cytokines secreted by astrocytes and microglia plays a particular role as mediators of this inflammatory response and they are thought to be among the many diverse signals that can trigger apoptosis in the brain following perinatal asphyxia and contribute to neuronal cell death. However, as elsewhere in the body, certain cytokines in the CNS might function early on to amplify the disease process and later on to attenuate it. The rapid synthesis of PGE₂ in response to cytokine and hypoxic stimulation may make it particularly useful in the diagnosis and surveillance of infants that has been exposed to birth asphyxia.

As described further herein (see particularly Example 7 below), it has now been found that PGE₂ is released in the brain as a result of perinatal asphyxia. This suggests that mPGES-1 is rapidly activated and involved in the response to severe hypoxia in mammals, such as humans and mice. The discovery of the role of the induced PGE₂ pathway in the response to hypoxia, such as perinatal asphyxia, provides a target for therapeutic intervention as well as a diagnostic tool, particularly for newborn infants that have been subjected to perinatal asphyxia.

### Mammal

In accordance with any aspect of the present invention the mammal or mammalian subject may be an adult, child or an infant, such as a neonate. The mammal or mammalian subject is preferably a human. In certain embodiments, the human may be of any age or of a particular age range, such as under 16 years of age, under ten years of age, 0 to 5 years of age and 0 to 24 months of age. In certain cases the subject is a human child having autonomic dysfunction disorders such as in PWS, CCHS or Rett's Syndrome. Thus in accordance with any aspect of the present invention, the subject may be a human (infant, child or adult) having familial dysautonomia or a human (infant, child or adult) with breathing and assosciated autonomic disturbances originating in the brainstem of unknown etiology.

In certain cases the subject is a human child (0-18 years of age) suffering from OSAS. The subject may be a human infant of 0-25 weeks postnatal age and 28-36 weeks gestational age. In certain embodiments the human may be an adult, such as over 18 years of age. The mammal may be an adult human suffering from sleep apnea (e.g. OSAS, snoring) and/or Parkinson's disease. As a result of studies described herein, there is an indication that elevated u-PGEM may be particularly important for increasing the susceptibility to and/or severity of apnea (including sleep apnea) among a sub-population of adults having OSAS and a body mass index (BMI) of no greater than 30. BMI is calculated by dividing a subject's weight in kg by the square of his or her height in metres. Thus, a subject having a BMI>30 is typically considered obese. In certain embodiments in accordance with any aspect of the invention the subject may be an adult human having a BMI>30.

### Induced PGE₂ Pathway

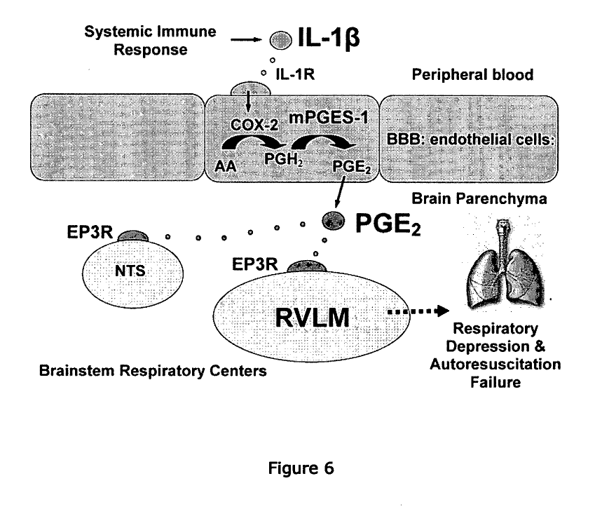

The present invention contemplates manipulation of the induced PGE₂ pathway for therapeutic treatment of breathing disorders as defined herein. The inventors have discovered that the induced PGE₂ pathway is implicated in causing increased apnea frequency and failure to autoresuscitate after a hypoxic event. The induced PGE₂ pathway is depicted in FIG. 6. During a systemic immune response, the pro-inflammatory cytokine IL-1β is released into the peripheral blood stream. It binds to its receptor (IL-1R) located on endothelial cells of the blood-brain barrier. Activation of IL-1R induces the synthesis of PGH₂ from arachidonic acid via COX-2 and the synthesis of PGE₂ from PGH₂ via the rate limiting enzyme mPGES-1. PGE₂ is released into the brain parenchyma and binds to EP3R located in respiratory control regions of the brainstem, e.g., nucleus of the solitary tract (NTS) and the rostral ventrolateral medulla (RVLM).

The present invention contemplates manipulation, such as pharmacological manipulation, of the induced PGE₂ pathway at one or more sites in order to block or reduce downstream effects on the respiratory control regions of the brainstem. The induced PGE₂ pathway may be inhibited at any point that has the effect of blocking or reducing downstream effects on the respiratory control regions of the brainstem. In particular, the induced PGE₂ pathway may be blocked by inhibiting COX-2, mPGES-1 and/or EP3R as further described herein.

### Inhibitor of the Induced PGE₂ Pathway

An inhibitor of the induced PGE₂ pathway has the ability to block or reduce downstream effects on the respiratory control regions of the brainstem. The inhibitor may act at any point in the induced PGE₂ pathway directly or indirectly. For example, the inhibitor may:
(a) directly interact with a polypeptide that participates in the pathway (an “induced PGE₂ pathway polypeptide”), for example a COX-2 polypeptide, an mPGES-1 polypeptide and/or an EP3R polypeptide;
(b) indirectly interacting with a polypeptide that participates in the pathway, for example by binding to and inhibiting an activator of a COX-2 polypeptide, an mPGES-1 polypeptide and/or an EP3R polypeptide; and/or
(c) interfering with expression of a gene that encodes an induced PGE₂ pathway polypeptide, for example down regulating expression (e.g. transcription and/or translation) of a COX-2-encoding gene, an mPGES-1-encoding gene and/or an EP3R-encoding gene.

### EP3R

An E-prostanoid receptor subtype 3 (EP3R) polypeptide has the ability to bind an EP3R agonist, such as PGE₂, and to signal downstream, such as signalling via a G-protein. The human and mouse EP3R amino acid sequences have previously been reported (84, the disclosure of which is expressly incorporated herein by reference). The human EP3R nucleotide sequence has been deposited in the GenBank database (Accession No. L26976, the disclosure of which is expressly incorporated herein by reference). An EP3R polypeptide preferably comprises or consists of the human EP3R amino acid sequence of SEQ ID NO: 2. However, an EP3R polypeptide may be a homologue from a non-human mammal, such as a mouse or other rodent. The EP3R polypeptide may be a variant or derivative of the human EP3R protein wherein one or more amino acids are altered by insertion, deletion or substitution. Preferably, the EP3R polypeptide comprises an amino acid sequence that has at least 70%, more preferably 80%, yet more preferably 90%, yet more preferably 95%, most preferably 99% amino acid identity to the full-length amino acid sequence of SEQ ID NO: 2, and has the ability to bind an EP3R agonist, such as PGE₂, and to signal downstream. In some embodiments, the EP3R polypeptide may be isolated.

Activation of human EP3R causes a decrease in [cAMP]ᵢ and modest increases in [Ca⁺⁺]ᵢ (84). Reduction of cAMP has been shown to decrease the firing amplitude and rate in respiration-related brainstem neurons and thus breathing activity (85). In neurons, activation of EP3R may hinder neurite extension via a protein kinase C-independent Rho-activation pathway (86, 87). Furthermore, EP3R are highly expressed in the kidney where EP3R activation exerts a vasoconstrictor effect (88).

An EP3R polypeptide may be an active portion which is less than the full-length EP3R polypeptide having the amino acid sequence of SEQ ID NO: 2, but which retains its essential biological activity. In particular, the active portion is capable of binding an EP3R agonist, such as PGE₂, and signalling downstream, such as signalling via a G-protein.

An EP3R-encoding gene may comprise a nucleotide sequence that encodes an EP3R polypeptide as defined herein. The EP3R-encoding gene may comprise a nucleotide sequence having at least 70%, more preferably 80%, yet more preferably 90%, yet more preferably 95%, most preferably 99% nucleotide sequence identity to the full-length nucleotide sequence of SEQ ID NO: 1.

### Inhibitor of EP3R

An inhibitor of EP3R prevents or reduces EP3R-mediated effects on brainstem respiratory control regions, such as preventing or reducing EP3R-mediated apnea, respiratory depression and/or autoresuscitation failure.

The invention contemplates the use of a number of different types of inhibitor of EP3R. For example, the inhibitor of EP3R may be an antagonist which binds to an EP3R polypeptide as defined herein and prevents or decreases agonist-induced (such as PGE₂-induced) downstream signalling (including G-protein-coupled signalling). Furthermore, the inhibitor may act indirectly by binding to and inhibiting an activator of an EP3R polypeptide. Also contemplated are inhibitors of EP3R that down regulate expression of an EP3R-encoding gene as defined herein (e.g. by inhibiting transcription and/or translation of an EP3R-encoding gene).

Examples of inhibitors that bind to an EP3R polypeptide include specific binding members, such as antibody molecules, and small molecules that compete with PGE₂ for binding to an EP3R polypeptide. Examples of inhibitors that down regulate expression of an EP3R-encoding gene include nucleic acid molecules that are complementary to an EP3R-encoding gene or a portion thereof and double stranded RNA corresponding to the sequence of a gene encoding EP3R or a fragment thereof. Inhibitors that down regulate expression of an EP3R-encoding gene also include ribozyme and/or triple helix agents. Further details of a number of different classes of inhibitor, including small molecules, specific binding members and nucleic acids are described herein.

### Small Molecule Inhibitors of EP3R

The present invention contemplates use of organic or inorganic compounds of up to around 2000 Daltons, such as 50-1000 Daltons, which bind to an EP3R polypeptide and prevent or reduce agonist-induced (such as PGE₂-induced) downstream signalling, such as G-protein signalling. The small-molecule inhibitor of EP3R may be an antagonist that binds to an EP3R polypeptide competitively, such that it competes for binding to the same site as PGE₂, or that binds non-competitively. The small-molecule EP3R antagonist will preferably be centrally acting (i.e. is able to cross the blood brain barrier). However, small-molecule EP3R antagonists that are not able to cross the blood brain barrier are also contemplated and may be delivered centrally, e.g. by intracerebroventricular (i.c.v.) administration.

The small-molecule EP3R antagonist may comprise (2E)-N-[(5-bromo-2-methoxyphenyl)sulfonyl]-3-[5-chloro-2-(2-naphthylmethyl)phenyl]acrylamide (L826266) or a pharmaceutically acceptable salt thereof.

Further small molecule EP3R antagonists may be identified using screening methods described further herein.

### Specific Binding Member Inhibitors of EP3R

In some embodiments, the inhibitor of EP3R may be a specific binding member which binds an EP3R polypeptide as defined herein and prevents or reduces agonist-induced (such as PGE₂-induced) downstream signalling, such as G-protein signalling.

In some embodiments, the specific binding member may be an antibody molecule. In other embodiments, the specific binding member may comprise an antigen-binding site within a non-antibody molecule, e.g. a set of CDRs in a non-antibody protein scaffold.

By “antibody molecule”, it is meant an immunoglobulin whether natural or partly or wholly synthetically produced. It has been shown that fragments of a whole antibody can perform the function of binding antigens. Thus reference to an antibody molecule covers a full antibody and also covers any polypeptide or protein comprising an antibody binding fragment.

Examples of binding fragments are (i) the Fab fragment consisting of VL, VH, CL and CH1 domains; (ii) the Fd fragment consisting of the VH and CH1 domains; (iii) the Fv fragment consisting of the VL and VH domains of a single antibody; (iv) the dAb fragment (55) which consists of a VH domain; (v) isolated CDR regions; (vi) F(ab′)₂ fragments, a bivalent fragment comprising two linked Fab fragments (vii) single chain Fv molecules (scFv), wherein a VH domain and a VL domain are linked by a peptide linker which allows the two domains to associate to form an antigen binding site (56-57); (viii) bispecific single chain Fv dimers (WO 93/11161) and (ix) “diabodies”, multivalent or multispecific fragments constructed by gene fusion (WO94/13804; 58). Fv, scFv or diabody molecules may be stabilised by the incorporation of disulphide bridges linking the VH and VL domains (59). Minibodies comprising a scFv joined to a CH3 domain may also be made (60).

### Nucleic Acid Inhibitors of EP3R

The present invention also includes the use of techniques known in the art for the down regulation of EP3R gene expression. These include the use RNA interference (RNAi).

In humans, EP3R is encoded by a gene having the nucleotide sequence of SEQ ID NO: 1. The human EP3R amino acid sequence is shown in SEQ ID NO: 2. The nucleotide sequence may be employed in the design of nucleic acid molecules that are capable of down regulating expression of an EP3R-encoding gene, as further described herein.

Small RNA molecules may be employed to regulate gene expression. These include targeted degradation of mRNAs by small interfering RNAs (siRNAs), post transcriptional gene silencing (PTGs), developmentally regulated sequence-specific translational repression of mRNA by micro-RNAs (miRNAs) and targeted transcriptional gene silencing.

A role for the RNAi machinery and small RNAs in targeting of heterochromatin complexes and epigenetic gene silencing at specific chromosomal loci has also been demonstrated. Double-stranded RNA (dsRNA)-dependent post transcriptional silencing, also known as RNA interference (RNAi), is a phenomenon in which dsRNA complexes can target specific genes of homology for silencing in a short period of time. It acts as a signal to promote degradation of mRNA with sequence identity. A 20-nt siRNA is generally long enough to induce gene-specific silencing, but short enough to evade host response. The decrease in expression of targeted gene products can be extensive with 90% silencing induced by a few molecules of siRNA.

In the art, these RNA sequences are termed “short or small interfering RNAs” (siRNAs) or “microRNAs” (miRNAs) depending in their origin. Both types of sequence may be used to down-regulate gene expression by binding to complimentary RNAs and either triggering mRNA elimination (RNAi) or arresting mRNA translation into protein. siRNA are derived by processing of long double stranded RNAs and when found in nature are typically of exogenous origin. Micro-interfering RNAs (miRNA) are endogenously encoded small non-coding RNAs, derived by processing of short hairpins. Both siRNA and miRNA can inhibit the translation of mRNAs bearing partially complimentary target sequences without RNA cleavage and degrade mRNAs bearing fully complementary sequences.

The siRNA ligands are typically double stranded and, in order to optimise the effectiveness of RNA mediated down-regulation of the function of a target gene, it is preferred that the length of the siRNA molecule is chosen to ensure correct recognition of the siRNA by the RISC complex that mediates the recognition by the siRNA of the mRNA target and so that the siRNA is short enough to reduce a host response.

miRNA ligands are typically single stranded and have regions that are partially complementary enabling the ligands to form a hairpin. miRNAs are RNA genes which are transcribed from DNA, but are not translated into protein. A DNA sequence that codes for a miRNA gene is longer than the miRNA. This DNA sequence includes the miRNA sequence and an approximate reverse complement. When this DNA sequence is transcribed into a single-stranded RNA molecule, the miRNA sequence and its reverse-complement base pair to form a partially double stranded RNA segment. The design of microRNA sequences is discussed in (61).

Typically, the RNA ligands intended to mimic the effects of siRNA or miRNA have between 10 and 40 ribonucleotides (or synthetic analogues thereof), more preferably between 17 and 30 ribonucleotides, more preferably between 19 and 25 ribonucleotides and most preferably between 21 and 23 ribonucleotides. In some embodiments of the invention employing double-stranded siRNA, the molecule may have symmetric 3′ overhangs, e.g. of one or two (ribo)nucleotides, typically a UU of dTdT 3′ overhang. Based on the disclosure provided herein, the skilled person can readily design of suitable siRNA and miRNA sequences, for example using resources such as Ambion's siRNA finder, see http://www.ambion.com/techlib/misc/siRNA_finder.html. siRNA and miRNA sequences can be synthetically produced and added exogenously to cause gene downregulation or produced using expression systems (e.g. vectors). In a preferred embodiment the siRNA is synthesized synthetically.

Longer double stranded RNAs may be processed in the cell to produce siRNAs (see for example (62)). The longer dsRNA molecule may have symmetric 3′ or 5′ overhangs, e.g. of one or two (ribo)nucleotides, or may have blunt ends. The longer dsRNA molecules may be 25 nucleotides or longer. Preferably, the longer dsRNA molecules are between 25 and 30 nucleotides long. More preferably, the longer dsRNA molecules are between 25 and 27 nucleotides long. Most preferably, the longer dsRNA molecules are 27 nucleotides in length. dsRNAs 30 nucleotides or more in length may be expressed using the vector pDECAP (63).

Another alternative is the expression of a short hairpin RNA molecule (shRNA) in the cell. shRNAs are more stable than synthetic siRNAs. A shRNA consists of short inverted repeats separated by a small loop sequence. One inverted repeat is complimentary to the gene target. In the cell the shRNA is processed by DICER into a siRNA which degrades the target gene mRNA and suppresses expression. In a preferred embodiment the shRNA is produced endogenously (within a cell) by transcription from a vector, such as an adenovirus vector of the invention. shRNAs may be produced within a cell by transfecting the cell with a vector encoding the shRNA sequence under control of a RNA polymerase III promoter such as the human H1 or 7SK promoter or a RNA polymerase II promoter. Alternatively, the shRNA may be synthesised exogenously (in vitro) by transcription from a vector. The shRNA may then be introduced directly into the cell. Preferably, the shRNA molecule comprises a partial sequence of an EP3R-encoding gene. Preferably, the shRNA sequence is between 40 and 100 bases in length, more preferably between 40 and 70 bases in length. The stem of the hairpin is preferably between 19 and 30 base pairs in length. The stem may contain G-U pairings to stabilise the hairpin structure.

siRNA molecules, longer dsRNA molecules or miRNA molecules may be made recombinantly by transcription of a nucleic acid sequence, preferably contained within a vector. Preferably, the siRNA molecule, longer dsRNA molecule or miRNA molecule comprises a partial sequence of an EP3R-encoding gene.

In one embodiment, the siRNA, longer dsRNA or miRNA is produced endogenously (within a cell) by transcription from a vector. The vector may be introduced into the cell in any of the ways known in the art. Optionally, expression of the RNA sequence can be regulated using a tissue specific promoter. In a further embodiment, the siRNA, longer dsRNA or miRNA is produced exogenously (in vitro) by transcription from a vector.

In one embodiment, the vector may comprise a full or partial nucleic acid sequence of an EP3R-encoding gene in both the sense and antisense orientation, such that when expressed as RNA the sense and antisense sections will associate to form a double stranded RNA. Preferably, the vector comprises the nucleic acid sequence of SEQ ID NO: 1; or a variant or fragment thereof. In another embodiment, the sense and antisense sequences are provided on different vectors. Preferably, the vector comprises the nucleic acid sequence of SEQ ID NO: 1, or a variant or fragment thereof.

Alternatively, siRNA molecules may be synthesized using standard solid or solution phase synthesis techniques which are known in the art. Linkages between nucleotides may be phosphodiester bonds or alternatives, for example, linking groups of the formula P(O)S, (thioate); P(S)S, (dithioate); P(O)NR′2; P(O)R′; P(O)OR6; CO; or CONR′2 wherein R is H (or a salt) or alkyl (1-12C) and R6 is alkyl (1-9C) is joined to adjacent nucleotides through —O— or —S—. Modified nucleotide bases can be used in addition to the naturally occurring bases, and may confer advantageous properties on siRNA molecules containing them.

For example, modified bases may increase the stability of the siRNA molecule, thereby reducing the amount required for silencing. The provision of modified bases may also provide siRNA molecules which are more, or less, stable than unmodified siRNA.

The term ‘modified nucleotide base’ encompasses nucleotides with a covalently modified base and/or sugar. For example, modified nucleotides include nucleotides having sugars which are covalently attached to low molecular weight organic groups other than a hydroxyl group at the 3′ position and other than a phosphate group at the 5′ position. Thus modified nucleotides may also include 2′ substituted sugars such as 2′-O-methyl-; 2-O-alkyl; 2-O-allyl; 2′-S-alkyl; 2′-S-allyl; 2′-fluoro-; 2′-halo or 2′-azido-ribose, carbocyclic sugar analogues α-anomeric sugars; epimeric sugars such as arabinose, xyloses or lyxoses, pyranose sugars, furanose sugars, and sedoheptulose.

Modified nucleotides are known in the art and include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N6-methyladenine, 4-acetylcytosine, 5-(carboxyhydroxylmethyl) uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1- methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N6-isopentenyladenine, uracil-5-oxyacetic acid methyl ester, psueouracil, 2-thiocytosine, 5-methyl-2 thiouracil, 2-thiouracil, 4-thiouracil, 5methyluracil, N-uracil-5-oxyacetic acid methylester, uracil 5-oxyacetic acid, queosine, 2-thiocytosine, 5-propyluracil, 5-propylcytosine, 5-ethyluracil, 5ethylcytosine, 5-butyluracil, 5-pentyluracil, 5-pentylcytosine, and 2,6,diaminopurine, methylpsuedouracil, 1-methylguanine, 1-methylcytosine.

Methods relating to the use of RNAi to silence genes in C. elegans, Drosophila, plants, and mammals are known in the art (WO 01/29058; WO 99/32619; 64-74, all of which are expressly incorporated herein by reference).

A ribozyme that down regulates expression of an EP3R-encoding gene is preferably specific for the RNA sequence of an EP3R-encoding gene, such as the EP3R-encoding gene having the DNA sequence of SEQ ID NO: 1. Ribozymes are nucleic acid molecules, actually RNA, which specifically cleave single-stranded RNA, such as mRNA, at defined sequences, and their specificity can be engineered. Hammerhead ribozymes may be preferred because they recognise base sequences of about 11-18 bases in length, and so have greater specificity than ribozymes of the Tetrahymena type which recognise sequences of about 4 bases in length, though the latter type of ribozymes are useful in certain circumstances. References on the use of ribozymes include Marschall, et al. 1994; Hasselhoff, 1988 and Cech, 1988.

mPGES-1

A microsomal prostaglandin E synthase-1 (mPGES-1) polypeptide has the ability to catalyse PGE₂ synthesis from PGH₂ in the presence of glutathione. mPGES-1 polypeptide preferably comprises or consists of the human mPGES-1 amino acid sequence of SEQ ID NO: 4. However, an mPGES-1 polypeptide may be a homologue from a non-human mammal, such as a mouse or other rodent. The mPGES-1 polypeptide may be a variant or derivative of the human mPGES-1 protein wherein one or more amino acids are altered by insertion, deletion or substitution. Preferably, the mPGES-1 polypeptide comprises an amino acid sequence that has at least 70%, more preferably 80%, yet more preferably 90%, yet more preferably 95%, most preferably 99% amino acid identity to the full-length amino acid sequence of SEQ ID NO: 4, and has the ability to catalyse PGE₂ synthesis from PGH₂ in the presence of glutathione. In some embodiments, the mPGES-1 polypeptide may be isolated.

The coding sequence of human mPGES1 cDNA is shown below as SEQ ID NO: 3. The full cDNA with untranslated 5′ and 3′ ends is available at GenBank accession No. NM_{—}004878.3

An mPGES-1 polypeptide may be an active portion which is less than the full-length mPGES-1 polypeptide having the amino acid sequence of SEQ ID NO: 4, but which retains its essential biological activity. In particular, the active portion has the ability to catalyse PGE₂ synthesis from PGH₂ in the presence of glutathione.

An mPGES-1-encoding gene may comprise a nucleotide sequence that encodes an mPGES-1 polypeptide as defined herein. The mPGES-1-encoding gene may comprise a nucleotide sequence having at least 70%, more preferably 80%, yet more preferably 90%, yet more preferably 95%, most preferably 99% nucleotide sequence identity to the full-length nucleotide sequence of SEQ ID NO: 3.

Inhibitor of mPGES-1

An inhibitor of mPGES-1 prevents or reduces mPGES-1-mediated synthesis of PGE₂. An inhibitor of mPGES-1 may prevent or reduce mPGES-1-mediated elevation of PGE₂ levels, particularly PGE₂ levels in blood brain barrier endothelial cells and/or brain parenchyma. By preventing or reducing PGE₂ synthesis, mPGES-1 inhibitors may ameliorate apnea, respiratory depression and/or autoresuscitation failure mediated by the induced PGE₂ pathway.

The invention contemplates the use of a number of different types of inhibitor of mPGES-1. For example, an inhibitor may bind to an mPGES-1 polypeptide as defined herein in order to disrupt its catalytic function, such inhibitors include competitive inhibitors which bind the active catalytic site of the mPGES-1 polypeptide and allosteric inhibitors which bind the mPGES-1 polypeptide at a site remote from the active catalytic site. Furthermore, the inhibitor may act indirectly by binding and inhibiting an activator of an mPGES-1 polypeptide. Also contemplated are inhibitors of mPGES-1 that down regulate expression of an mPGES-1-encoding gene (e.g. by inhibiting transcription and/or translation of an mPGES-1-encoding gene).

Examples of inhibitors that bind to an mPGES-1 polypeptide include specific binding members, such as antibody molecules, and small molecules that bind to an mPGES-1 polypeptide competitively or non-competitively. Examples of inhibitors that down regulate expression of an mPGES-1-encoding gene include nucleic acid molecules that are complementary to an mPGES-1-encoding gene or a portion thereof and double stranded RNA corresponding to the sequence of a gene encoding mPGES-1 or a fragment thereof. Inhibitors that down regulate expression of an mPGES-1-encoding gene also include ribozyme and/or triple helix agents. Further details of a number of different classes of inhibitor, including small molecules, specific binding members and nucleic acids are described herein.

Small Molecule Inhibitors of mPGES-1

A small molecule mPGES-1 inhibitor may bind to an mPGES-1 polypeptide and prevent or limit mPGES-1 polypeptide conversion of a cyclic endoperoxide substrate into a product which is the 9-keto, 11α hydroxyl form of the substrate. The small molecule may bind to the active site of an mPGES-1 polypeptide or a remote site, and may bind reversibly or irreversibly.

A number of compounds have been found to inhibit the mPGES-1 enzyme, including leukotriene C4, NS-398, sulindac sulfide with IC₅₀ values of 5, 20 and 80 μM, respectively (75, the disclosure of which is expressly incorporated herein by reference). Also, 15-deoxy-Δ12,14-PGJ₂, arachidonic acid, docosahexaenoic acid, eicosapentaenoic acid and 3-[tert-Butylthio-1-(4-chlorobenzyl)-5-isopropyl-1H-indol-2-yl]-2,2-dimethylpropionic acid (MK-886) were all reported to inhibit mPGES with similar IC₅₀ values of 0.3 μM (76-77).

Further small molecule mPGES-1 inhibitors may be identified using screening methods described further herein.

Specific Binding Member Inhibitors of mPGES-1

In some embodiments, the mPGES-1 inhibitor may be specific binding member which binds an mPGES-1 polypeptide as defined herein and prevents or reduces mPGES-1-mediated conversion of a cyclic endoperoxide substrate into a product which is the 9-keto, 11α hydroxyl form of the substrate.

The specific binding member inhibitor of mPGES-1 may be an antibody molecule. Different types of antibody molecules are described above in relation to specific binding member inhibitors of EP3R. The antibody molecule may be as described therein, except that the antibody molecule will bind an mPGES-1 polypeptide rather than an EP3R polypeptide.

Nucleic Acid Inhibitors of mPGES-1

The present invention also contemplates inhibitors that down regulate expression of an mPGES-1-encoding gene.

In humans, mPGES-1 is encoded by a gene having the nucleotide sequence of SEQ ID NO: 3. The human mPGES-1 amino acid sequence is shown in SEQ ID NO: 4. The nucleotide sequence may be employed in the design of nucleic acid molecules that are capable of down regulating expression of an mPGES-1-encoding gene, as further described above in relation to inhibitors of EP3R, except that nucleic acid molecules will down regulate expression of an mPGES-1-encoding gene rather than an EP3R-encoding gene. References to a sequence, partial sequence or complementary sequence of an EP3R-encoding gene, therefore, apply to a sequence, partial sequence or complementary sequence of an mPGES-1-encoding gene, mutatis mutandis.

### COX-2

A cyclooxygenase-2 (COX-2) polypeptide has the ability to catalyse PGH₂ synthesis from arachidonic acid. The amino acid sequence of human COX-2 has been deposited at GenBank accession No. NP_{—}000954 (which is expressly incorporated herein by reference) and also shown below as SEQ ID NO: 6. A COX-2 polypeptide preferably comprises or consists of the human COX-2 amino acid sequence of SEQ ID NO: 6. However, a COX-2 polypeptide may be a homologue from a non-human mammal, such as a mouse or other rodent. The COX-2 polypeptide may be a variant or derivative of the human COX-2 protein wherein one or more amino acids are altered by insertion, deletion or substitution. Preferably, the COX-2 polypeptide comprises an amino acid sequence that has at least 70%, more preferably 80%, yet more preferably 90%, yet more preferably 95%, most preferably 99% amino acid identity to the full-length amino acid sequence of SEQ ID NO: 6, and has the ability to catalyse PGH₂ synthesis from arachidonic acid. In some embodiments, the COX-2 polypeptide may be isolated.

A COX-2 polypeptide may be an active portion which is less than the full-length COX-2 polypeptide having the amino acid sequence of SEQ ID NO: 6, but which retains its essential biological activity. In particular, the active portion has the ability to catalyse PGH₂ synthesis from arachidonic acid.

The cDNA sequence of human COX-2 has been deposited at GenBank (accession No. NM_{—}000963, which is expressly incorporated herein by reference) and is shown below as SEQ ID NO: 5. The coding sequence is from nucleotides 135 to 1949, marked bold.

A COX-2-encoding gene may comprise a nucleotide sequence that encodes an COX-2 polypeptide as defined herein. The COX-2-encoding gene may comprise a nucleotide sequence having at least 70%, more preferably 80%, yet more preferably 90%, yet more preferably 95%, most preferably 99% nucleotide sequence identity to the coding region of the nucleotide sequence of SEQ ID NO: 5 or of the coding region thereof (nucleotides 135 to 1949 of SEQ ID NO: 5).

### Selective Inhibitor of COX-2

A selective inhibitor of COX-2 prevents or reduces COX-2-mediated synthesis of PGH₂. A selective inhibitor of COX-2 may prevent or reduce COX-2-mediated elevation of PGH₂ levels and thereby ameliorate apnea, respiratory depression and/or autoresuscitation failure mediated by the induced PGE₂ pathway.

Furthermore, a selective inhibitor of COX-2 has greater inhibitory activity against COX-2 as compared with its inhibitory activity against COX-1. The selectivity of the inhibitor of COX-2 will generally decrease adverse effects associated with non-selective COX inhibition, such as effects caused by inhibition of important constitutive COX-1 activity. A selective inhibitor of COX-2 may have 2-fold or more, such as 5 or 10-fold greater inhibitory activity against COX-2 than COX-1. Thus, the IC₅₀ value of the selective inhibitor of COX-2 may be 2-fold lower, preferably 5-fold or 10-fold lower than the IC₅₀ value of the same inhibitor for COX-1.

The invention contemplates the use of a number of different types of selective inhibitor of COX-2. For example, an inhibitor may bind to a COX-2 polypeptide as defined herein in order to disrupt its catalytic function, such inhibitors include competitive inhibitors which bind the active catalytic site of the COX-2 polypeptide and allosteric inhibitors which bind the COX-2 polypeptide at a site remote from the active catalytic site. Furthermore, the inhibitor may act indirectly by binding and inhibiting an activator of a COX-2 polypeptide. Also contemplated are inhibitors of COX-2 that down regulate expression of a COX-2-encoding gene (e.g. by inhibiting transcription and/or translation of an COX-2-encoding gene).

Examples of inhibitors that bind to a COX-2 polypeptide include specific binding members, such as antibody molecules, and small molecules that bind to a COX-2 polypeptide competitively or non-competitively. Examples of inhibitors that down regulate expression of a COX-2-encoding gene include nucleic acid molecules that are complementary to a COX-2-encoding gene or a portion thereof and double stranded RNA corresponding to the sequence of a gene encoding a COX-2 polypeptide or a fragment thereof. Inhibitors that down regulate expression of a COX-2-encoding gene also include ribozyme and/or triple helix agents. Further details of a number of different classes of inhibitor, including small molecules, specific binding members and nucleic acids are described herein.

### Small Molecule Inhibitors of COX-2

A small molecule selective inhibitor of COX-2 may bind to a COX-2 polypeptide and prevent or decrease COX-2-mediated conversion of arachidonic acid into PGH₂. The small molecule may bind to the active catalytic site of a COX-2 polypeptide or a remote site, and may bind reversibly or irreversibly.

A large number of compounds that act as selective inhibitors of COX-2 have been described. One exemplary class of COX-2 selective inhibitors are drugs known as “coxibs”.

In some embodiments the small molecule selective inhibitor of COX-2 may comprise 4-(5-methyl-3-phenylisoxazol-4-yl)benzenesulfonamide (valdecoxib) or a pharmaceutically acceptable salt thereof; 4-[5-(4-methylphenyl)-3-(trifluoromethyl)pyrazol-1-yl]benzenesulfonamide (celecoxib) or a pharmaceutically acceptable salt thereof; and/or 4-(4-methylsulfonylphenyl)-3-phenyl-5H-furan-2-one (rofecoxib) or a pharmaceutically acceptable salt thereof.

A large number of COX-2 inhibitors, useful in accordance with the invention, have been described previously (see 94, the disclosure of which is expressly incorporated herein by reference, for a review of the pharmacology of COX, particularly COX-2, inhibition).

Further small molecule selective inhibitors of COX-2 may be identified using screening methods described further herein.

### Specific Binding Member Inhibitors of COX-2

In some embodiments, the selective inhibitor of COX-2 may be specific binding member which binds a COX-2 polypeptide as defined herein and prevents or reduces COX-2-mediated conversion of arachidonic acid into PGH₂.

The specific binding member inhibitor of COX-2 may be an antibody molecule. Different types of antibody molecules are described above in relation to specific binding member inhibitors of EP3R. The antibody molecule may be as described therein, except that the antibody molecule will bind a COX-2 polypeptide rather than an EP3R polypeptide. Preferably, the specific binding member inhibitor of COX-2 will not cross-react with a COX-1 polypeptide.

### Nucleic Acid Inhibitors of COX-2

The present invention also contemplates inhibitors that down regulate expression of a COX-2-encoding gene.

In humans, COX-2 is encoded by a gene having the nucleotide sequence of SEQ ID NO: 5. The human COX-2 amino acid sequence is shown in SEQ ID NO: 6. The nucleotide sequence may be employed in the design of nucleic acid molecules that are capable of down regulating expression of a COX-2-encoding gene, as further described above in relation to inhibitors of EP3R, except that nucleic acid molecules will down regulate expression of a COX-2-encoding gene rather than an EP3R-encoding gene. References to a sequence, partial sequence or complementary sequence of an EP3R-encoding gene, therefore, apply to a sequence, partial sequence or complementary sequence of a COX-2-encoding gene, mutatis mutandis.

### Therapy

The present invention contemplates both therapeutic and prophylactic treatment of breathing disorders as defined herein. The treatment may reduce susceptibility of a mammal to a breathing disorder and/or fully or partially reverse one or more clinical aspects of a breathing disorder in a mammal. For example, the invention contemplates regularising the breathing of a patient experiencing apnea. Also contemplated is the enhancement of autoresuscitation following a hypoxic event.

In preferred embodiments, the mammal may be a patient determined to be at risk of a breathing disorder as defined herein. For example, a human infant suffering from an infection, especially an infection causing elevated IL-1β levels, may be treated with an agent comprising: an inhibitor of EP3R; an inhibitor of mPGES-1; and/or a selective inhibitor of COX-2, in order to reduce the likelihood of and severity of apnea.

### Formulations

The present invention contemplates a variety of pharmaceutical compositions of an inhibitor as defined herein. A pharmaceutical composition will generally comprise one or more pharmaceutically acceptable salts, carriers or excipients. Furthermore, pharmaceutical compositions comprising more than one inhibitor as defined herein are contemplated. For example, a composition may comprise two or more agents selected from: an inhibitor of EP3R; an inhibitor of mPGES-1; and a selective inhibitor of COX-2. Alternatively, if more than one inhibitor is employed, the agents may be formulated in separate compositions for simultaneous or sequential delivery.

### Modes of Administration

Any suitable route of administration may be employed in accordance with the present invention. Typically, a composition comprising an inhibitor as defined herein may be administered orally, rectally, intranasally, by intravenous, intramuscular, subcutaneous, intraperitoneal or intracerebroventricular injection, transcutaneous patch or minipump. In the case of a composition comprising an inhibitor of EP3R that is not able to cross the blood brain barrier, intracerebroventricular injection may be preferred.

### Assessment and Diagnosis

The present invention contemplates methods of assessing susceptibility to, or presence of, a breathing disorder in a mammal by detecting one or more markers of the induced PGE₂ pathway in a sample from the mammal. A subject found to have a breathing disorder or an increased risk of a breathing disorder may then be treated with an inhibitor as defined herein.

A number of methods are contemplated for assessing whether a patient has increased activity of the induced PGE₂ pathway. In some embodiments the level of PGE₂ or a metabolite thereof is detected in a sample from the subject and is compared to a control level. The control level is preferably a pre-determined “normal” range. For example, the control level may be the level of PGE₂ or the metabolite thereof that is found in a similar sample from a healthy control. The control level may represent a range of values previously determined or reported for healthy control subjects, and may represent an average value obtained from a population.

PGE₂ and/or one or more of its metabolites may be measured in a biological sample as defined further herein. There are a number of PGE₂ metabolites, most of which can be detected by LC-MS/MS (Liquid chromatography triple quadrupole mass spectrometer) (105, the disclosure of which is incorporated herein by reference in its entirety).

Examples of PGE₂ metabolites in accordance with the invention include: 7alpha-hydroxy-5,11-diketo-2,3,4,5,20-penta-19-carboxyprostanoic acid and 13,14-dihydro-15-keto metabolites of the E and F series. PGE₂ and/or one or more PGE₂ metabolites (including 7alpha-hydroxy-5,11-diketo-2,3,4,5,20-penta-19-carboxyprostanoic acid and 13,14-dihydro-15-keto metabolites of the E and F series) may be measured by any suitable technique for the sample concerned.

PGE₂ metabolites, in accordance with the present invention, and techniques for detection and measurement thereof are also described in (106, the disclosure of which is incorporated herein by reference in its entirety).

Particular examples of assays for the measurement of PGE₂ and metabolites thereof include: enzyme immuno assays (EIA) as described in further detail in the Examples section below. EIA kits are available commercially and permit sensitive detection of individual compounds.

As a further example, measurement or detection of PGE₂ and/or one or more metabolites thereof (including 7alpha-hydroxy-5,11-diketo-2,3,4,5,20-penta-19-carboxyprostanoic acid and 13,14-dihydro-15-keto metabolites of the E and F series) may employ LC.MS/MS and/or triple quad mass spectrometry (also known as triple quadrupole (QQQ)). The use of triple quad mass spectrometry may be preferred in certain situations due to the ability of such analysis to detect femto/picomolar concentrations of compounds. A tandem quadrupole (triple quadrupole) instrument for quantification of known metabolites and peptides (such as PGE₂ and/or one or more metabolites thereof). This instrument can be used for quantitative pathway analysis of the arachidonic acid cascade. Furthermore, this instrument will be used for quantitative validation of peptides in clinical material and quantitative validation of metabolites identified in metabolomics as different between different clinical materials. The proposed instrumentation will be connected to an Ultra Performance Liquid Chromatograph (UPLC) via an electro spray ionization interface (ESI). The use of small particle size particles (<1.8 μm) in liquid chromatography dramatically narrows the chromatographic peak width, typically 3-5 seconds (UPLC) compared to 30-60 seconds (conventional LC). This enables better separation and hence more compounds can be separated in a shorter time. In a triple quadrupole mass spectrometer, the molecular ion of a particular metabolite is selected in the first quadrupole, fragmentation of the metabolite is induced in a collision cell with a collision gas. A particular “daughter ion” is selected in the second quadrupole yielding an electronic transition trace (reaction monitoring). This daughter ion constitutes a very compound specific tracer, since distinct metabolites/peptides will fragment differently. Typically ˜100 traces can be monitored simultaneously (multiple reaction monitoring, MRM) enabling specific and sensitive quantification of many metabolites in one analysis. Preferably, the method of the invention comprises measurement of one or more PGE₂ metabolites in a urine sample and employs triple quadrupole mass spectrometry. A particularly preferred assay for measurement of urinary PGE₂ metabolites (u-PGEM) is as described in Example 8. In some cases the method of the invention comprises measurement of one or more PGE₂ metabolites in a urine sample, which method further comprises determining the concentration of creatinine in the urine sample, wherein the urinary level of PGE₂ is the level relative to the urinary creatinine level.

Comparing the level of PGE₂ or a metabolite thereof in the sample with a control level may be accomplished by consulting a chart, database or literature reporting a predetermined control value or range of control values. In some cases, for example when no predetermined control value is available, comparing the sample level with a control level may comprise detecting the level of PGE₂ or a metabolite thereof in a control sample from a healthy subject sequentially or in parallel with detecting the level of PGE₂ or a metabolite thereof in the sample from the subject under investigation.

An elevated PGE₂ level, or PGE₂ metabolite level, compared with the control level is considered to indicate the presence of or an increased risk of a breathing disorder, such as increased apnea frequency.

Data described below provide evidence that PGE₂ metabolites may be used as useful indicator to estimate the degree of asphyxia an infant has experienced at around the time of birth (“perinatal asphyxia”) and/or the presence or severity of hypoxic ischemic encephalopathy (HIE) in a mammalian subject. An elevated PGE₂ level, or PGE₂ metabolite level, particularly in a sample taken from the subject within seven days, such as within 96, 48, 24, 12, 6, 4, 3 or 2 hours or within 60, 30, 20, 10 or 5 minutes, of birth of the subject, as compared with the control level has been found to be predictive of the presence of HIE in the mammalian subject and/or to indicate that the subject has been subjected to perinatal asphyxia. The degree of elevation of PGE₂ or a metabolite thereof compared with a control level has been found to correlate with the degree of perinatal asphyxia and/or the degree of severity of HIE and therefore the likely neurological outcome of the subject.

The methods of the invention are thus useful in the estimation of prognosis and long-term neurological outcome and thus valuable to help immediate decisions regarding treatment.

Experimental results indicate that the half-life of PGE₂ may, in some cases, be about 12-18 hours. PGE₂ and metabolites thereof may persist and may be measured even after more than 72 hours. Half time for PGE2 degradation various considerably pending on the cellular environment. Half-life of PGE₂ can vary from a few minutes to several hours. When evaluating PGE2 produced in the body and secreted in urine or other body fluids it is important to also measure its metabolites.

In some embodiments the level of PGE₂ or a metabolite thereof in the sample is compared with a reference level of PGE₂ or a metabolite thereof. The reference level may be other than a control level. For example, the reference level may be a value or range of values indicative of a breathing disorder as defined herein or perinatal asphyxia or HIE in a mammalian subject. In such cases, a level of PGE₂, or metabolite thereof, at about the reference level or within the reference range of values indicates: the presence of or an increased risk of a breathing disorder as defined herein; the degree of asphyxia an infant has experienced during birth and/or the presence or severity of HIE in the subject. The reference level may be a value or range of values associated with a particular severity or stage of: a breathing disorder; asphyxia an infant has experienced during birth; and/or HIE in the subject.

In some embodiments the method includes assessing whether a patient has increased activity of the induced PGE₂ pathway by detecting the expression of an mPGES-1-encoding gene. This may include measuring levels of mRNA of an mPGES-1-encoding gene, for example using quantitative, semi-quantitative or real time PCR-based methods. Elevated expression of an mPGES-1-encoding gene may indicate increased risk of a breathing disorder. Other methods for assessing whether a patient has increased activity of the induced PGE₂ pathway include detecting elevated PGH₂ levels, increased COX-2 gene expression and/or increased IL-1β levels. The present invention contemplates detecting one or more markers of increased induced PGE₂ pathway activity. For example, detecting of PGE₂ levels may be combined with detection of PGH₂ levels, mPGES-1 expression, COX-2 expression and/or IL-1β levels.

In some embodiments, the method may involve identifying one or more mutations in a gene encoding mPGES-1, COX-2 and/or EP3R. For example, a single nucleotide polymorphism (SNP) in a gene encoding mPGES-1, COX-2 and/or EP3R may be linked to an increased susceptibility to a breathing disorder as defined herein.

### Sample

The sample may be a liquid sample such as a CSF sample, a blood sample, a urine sample or a non-liquid sample such as a biopsy tissue sample. Preferably, the sample is a CSF, urine or blood sample. In certain embodiments, a urine sample is particularly preferred.

The sample may be taken from a mammalian subject, such as a human subject at a predetermined time point after an actual or suspected cause of or onset of a condition as specified herein. For example, a sample may be taken from a human infant within 96, 48, 24, 12, 6, 4, 3 or 2 hours or within 60, 30, 20, 10 or 5 minutes, of birth of the subject or of admission to hospital or presentation to a clinician. In some cases the sample may be a human urine sample which has been stored at reduced temperature (e.g. at around 4° C. or at between −80° C. and −20° C.).

### Infection Markers

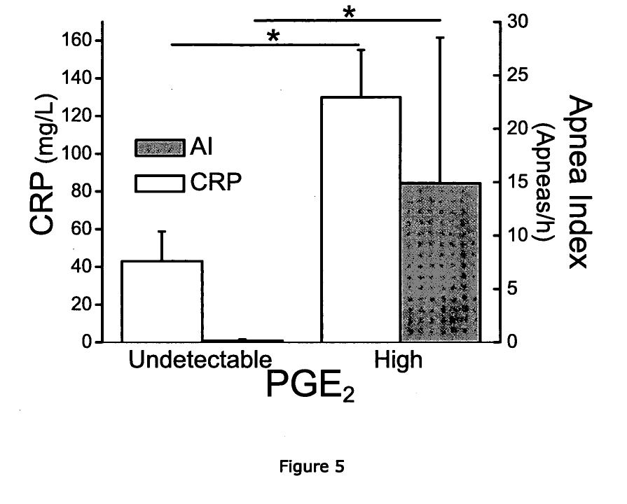

The present inventors have discovered that PGE₂ levels, CRP and apnea index are correlated (see FIG. 5). In some embodiments the method of diagnosis may additionally comprise detecting the level of an infection-related marker. For example, the level of CRP may be assessed in a sample, preferably a blood or urine sample, from the patient. An elevated level of an infection marker compared with a control level may indicate enhanced risk of a breathing disorder, particularly when combined with an elevated level of PGE₂ or other marker of increased activity of the induced PGE₂ pathway.

The control level is preferably a pre-determined “normal” range. For example, the control level may be the level of CRP that is found in a similar sample from a healthy control. The control level may represent a range of values previously determined or reported for healthy control subjects, and may represent an average value obtained from a population.

Comparing the level of CRP in the sample with a control level may be accomplished by consulting a chart, database or literature reporting a predetermined control value or range of control values. In some cases, for example when no predetermined control value is available, comparing the sample level with a control level may comprise detecting the level of CRP in a control sample from a healthy subject sequentially or in parallel with detecting the level of CRP in the sample from the subject under investigation.

An elevated CRP level compared with the control level is considered to indicate the presence of or an increased risk of a breathing disorder, such as increased apnea frequency.

In some embodiments the level of CRP in the sample is compared with a reference level of CRP. The reference level may be other than a control level. For example, the reference level may be a value or range of values indicative of a breathing disorder as defined herein. In which case, a level of CRP at about the reference level or within the reference range of values indicates the presence of or an increased risk of a breathing disorder as defined herein. The reference level may be a value or range of values associated with a particular severity or stage of a breathing disorder as defined herein.

Furthermore, measurement of PGE₂, or metabolites thereof, may be used to complement, or as an alternative to, the measurement of CRP or high-sensitive CRP (hsCRP) as an inflammatory marker.

### Screening Methods

The present invention contemplates identifying substances for use in treating a breathing disorder in a mammal. Accordingly, a method for identifying a substance for use in treating a breathing disorder in a mammal may comprise assaying a test substance for the ability to inhibit the induced PGE₂ pathway, for example a test substance which acts as an inhibitor of EP3R, an inhibitor of mPGES-1 and/or a selective inhibitor of COX-2,
wherein inhibition of the induced PGE₂ pathway indicates that the test substance is a substance for use in treating a breathing disorder in a mammal.

A test substance, which may be a candidate compound or composition, may inhibit the induced PGE₂ pathway by:
(a) directly interacting with a polypeptide that participates in the pathway (an “induced PGE₂ pathway polypeptide”), for example a COX-2 polypeptide, an mPGES-1 polypeptide and/or an EP3R polypeptide;
(b) indirectly interacting with a polypeptide that participates in the pathway, for example by binding to and inhibiting an activator of a COX-2 polypeptide, an mPGES-1 polypeptide and/or an EP3R polypeptide; and/or
(c) down regulating expression of a gene that encodes an induced PGE₂ pathway polypeptide, for example down regulating expression (e.g. transcription and/or translation) of a COX-2-encoding gene, an mPGES-1-encoding gene and/or an EP3R-encoding gene.

### Screening for Inhibitors of Polypeptides

Determination of the ability of a test substance to interact and/or bind with an induced PGE₂ pathway polypeptide may be used to identify that test substance as a possible inhibitor of the induced PGE₂ pathway. The method may comprise detecting or observing interaction or binding, and then using that test substance in a further assay method to determine whether it inhibits induced PGE₂ pathway polypeptide activity, for example enzyme activity or receptor-mediated signalling.

The precise format of assays of the invention may be varied by those of skill in the art using routine skill and knowledge. For example, interaction between polypeptides or peptides may be studied in vitro by labelling one with a detectable label and bringing it into contact with the other which has been immobilised on a solid support. Suitable detectable labels include ³⁵S-methionine which may be incorporated into recombinantly produced peptides and polypeptides. Recombinantly produced peptides and polypeptides may also be expressed as a fusion protein containing an epitope which can be labelled with an antibody.

The protein or peptide that is immobilized on a solid support may be immobilized using an antibody against that protein bound to a solid support or via other technologies which are known per se. A preferred in vitro interaction may utilise a fusion protein including glutathione-S-transferase (GST). This may be immobilized on glutathione agarose beads. In an in vitro assay format of the type described above a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis. Alternatively, the beads may be rinsed to remove unbound protein and the amount of protein which has bound can be determined by counting the amount of label present in, for example, a suitable scintillation counter.

Generally, the identification of ability of a test substance to bind or interact with an induced PGE₂ pathway polypeptide and its identification as a potential PGE₂ pathway inhibitor is followed by one or more further assay steps involving determination of whether or not the test substance is able to inhibit induced PGE₂ pathway polypeptide activity. Naturally, assays involving determination of ability of a test substance to inhibit an induced PGE₂ pathway polypeptide may be performed where there is no knowledge about whether the test substance can bind or interact with the induced PGE₂ pathway polypeptide, but a prior binding/interaction assay may be used as a screen to test a large number of compounds, reducing the number of potential inhibitors to a more manageable level for a functional assay involving determination of ability to inhibit the induced PGE₂ pathway polypeptide activity.

Assay methods for determining whether a test substance acts as an inhibitor of an induced PGE₂ pathway polypeptide, in particular COX-2, mPGES-1 and EP3R assays are described further herein.

Combinatorial library technology (78) provides an efficient way of testing a potentially vast number of different substances for ability to modulate activity of a polypeptide.

The amount of test substance or compound which may be added to an assay of the invention will normally be determined by trial and error depending upon the type of compound used. Typically, from about 0.1 nM to 10 μM concentrations of a test compound (e.g. putative inhibitor) may be used. Greater concentrations may be used when a peptide is the test substance. Compounds which may be used may be natural or synthetic chemical compounds used in drug screening programmes. Extracts of plants which contain several characterised or uncharacterised components may also be used. Other inhibitor or candidate inhibitor compounds may be based on modelling the 3-dimensional structure of a polypeptide or peptide fragment and using rational drug design to provide potential inhibitor compounds with particular molecular shape, size and charge characteristics.

### Screening for Inhibitors of Gene Expression

An inhibitor of the induced PGE₂ pathway may inhibit the pathway by interfering with expression of a gene that encodes an induced PGE₂ pathway polypeptide, for example a COX-2-encoding gene, an mPGES-1-encoding gene and/or an EP3R-encoding gene. Accordingly, assay methods of the invention may comprise identifying a test substance as a substance for use in treating a breathing disorder in a mammal, wherein the method comprises screening for a substance able to reduce or inhibit expression of a gene encoding an induced PGE₂ pathway polypeptide, comprising:
(a) contacting DNA containing the promoter of said gene with a test substance, wherein the promoter is operably linked to a gene;
(b) determining the level of gene expression from the promoter; and
(c) comparing said level of gene expression in the presence of the test substance with the level of gene expression in the absence of the test substance in comparable conditions,
wherein a reduced level of gene expression in the presence of the test substance indicates that the test substance is able to inhibit expression of the gene encoding an induced PGE₂ pathway polypeptide.

The method may further comprise identifying the test substance as an inhibitor of expression of the gene encoding an induced PGE₂ pathway polypeptide, i.e. as a substance for use in treating a breathing disorder in a mammal.

Thus, step (c) may comprise detecting a reduced level of gene expression in the presence of the test substance compared with the level of gene expression in the absence of the test substance in comparable conditions,
whereby the test substance is identified as a substance for use in treating a breathing disorder in a mammal.

The method may comprise contacting an expression system, such as a host cell containing the gene promoter operably linked to a gene with the test substance, and determining expression of the gene. The gene may be a gene that encodes an induced PGE₂ pathway polypeptide or it may be a heterologous gene, e.g. a reporter gene. A “reporter gene” is a gene whose encoded product may be assayed following expression, i.e. a gene which “reports” on promoter activity.

By “promoter” is meant a sequence of nucleotides from which transcription may be initiated of DNA operably linked downstream (i.e. in the 3′ direction on the sense strand of double-stranded DNA). The promoter of a gene may comprise or consist essentially of a sequence of nucleotides 5′ to the gene in the human chromosome, or an equivalent sequence in another species, such as a rat or mouse.

The level of promoter activity is quantifiable for instance by assessment of the amount of mRNA produced by transcription from the promoter or by assessment of the amount of protein product produced by translation of mRNA produced by transcription from the promoter. The amount of a specific mRNA present in an expression system may be determined for example using specific oligonucleotides which are able to hybridise with the mRNA and which are labelled or may be used in a specific amplification reaction such as the polymerase chain reaction (PCR).

Use of a reporter gene facilitates determination of promoter activity by reference to protein production. The reporter gene preferably encodes an enzyme which catalyses a reaction that produces a detectable signal, preferably a visually detectable signal, such as a coloured product. Many examples are known, including β-galactosidase and luciferase. β-galactosidase activity may be assayed by production of blue colour on substrate, the assay being by eye or by use of a spectrophotometer to measure absorbance. Fluorescence, for example that produced as a result of luciferase activity, may be quantified using a spectrophotometer. Radioactive assays may be used, for instance using chloramphenicol acetyltransferase, which may also be used in non-radioactive assays. The presence and/or amount of gene product resulting from expression from the reporter gene may be determined using a molecule able to bind the product, such as an antibody or fragment thereof. The binding molecule may be labelled directly or indirectly using any standard technique.

A promoter construct may be introduced into a cell line using any suitable technique to produce a stable cell line containing the reporter construct integrated into the genome. The cells may be grown and incubated with test compounds for varying times. The cells may be grown in 96 well plates to facilitate the analysis of large numbers of compounds. The cells may then be washed and the reporter gene expression analysed. For some reporters, such as luciferase the cells will be lysed then analysed.

Those skilled in the art are aware of a multitude of possible reporter genes and assay techniques which may be used to determine gene activity. For more examples, see Sambrook and Russell, Molecular Cloning: a Laboratory Manual: 3rd edition, 2001, Cold Spring Harbor Laboratory Press.

### COX-2 Assays

The present invention contemplates assay methods for determining whether a test substance, which may be a candidate compound or composition, has COX-2 selective inhibitory activity, whereby a test substance determined to have COX-2 selective inhibitory activity is identified as a substance for use in treating a breathing disorder.

In some embodiments the assay method comprises:
contacting a COX-2 polypeptide with a test substance and arachidonic acid, under conditions in which arachidonic acid would be converted to PGH₂ by COX-2 in the absence of the test substance; and
determining the level of PGH₂ production in the presence of the test substance compared with a control level of PGH₂ production in the absence of the test substance,
wherein a lower level of PGH₂ production in the presence of the test substance compared with said control level indicates that the test substance is a substance for use in treating a breathing disorder in a mammal.

Methods for identifying inhibitors of COX-2 include those described previously (89, 90, 91, all of which are expressly incorporated herein by reference). A candidate compound or composition found to inhibit COX-2 may be subjected to further testing as described herein, such as in vivo testing, in order to determine whether the compound or composition has the ability to treat a breathing disorder in a mammal.

A number of COX-2 inhibitor screening kits are commercially available. For example, Cayman Chemicals product No. 560131 “COX Inhibitor Screening Assay” provides the necessary cofactors of human COX-2 and the detection is based on SnCl₂ reduction of PGH₂ into mainly PGF2α. (see http://www.caymanchem.com/app/template/Product.vm/catalog/560131/a/z).

There are several other alternatives for detection of produced PGH₂, e.g. PGH₂ can, after treatment with iron chloride, be converted into 12-HHT and malondialdehyde, both of which can be measured in a high throughput manner or the peroxidase activity of COX-2 can be used, e.g. as described in the kit provided also by Cayman chemicals (see: http://www.caymanchem.com/app/template/Product.vm/catalog/760111/a/z).

The method normally comprises incubating the test substance or test substance with the enzyme and a substrate for the enzyme. The substrate may be a physiological substrate such as arachidonic acid, or it may be a modified or non-physiological substrate, such as a substrate designed to give rise to a detectable (e.g. coloured) product in the enzymatic reaction.

The order in which the COX-2 polypeptide is contacted with the test substance and with the substrate, such as arachidonic acid, may be varied. For example, the COX-2 polypeptide may be first incubated with the test substance and then contacted with substrate, or vice versa.

Thus, production of the product in the presence of the test substance may be compared with production of the product in the absence of the test substance. A lower level of product, or a lower rate of product formation indicates that the test substance inhibits the enzyme activity.

A further possibility for an assay for inhibitors is testing ability of a substance to affect PGH₂ production by a suitable cell line expressing COX-2 (either naturally or recombinantly). An assay according to the present invention may be performed in a cell line such as a yeast strain in which the relevant polypeptides or peptides are expressed from one or more vectors introduced into the cell.

A still further possibility for an assay is testing ability of a substance to affect PGH₂ production by an impure protein preparation including COX-2 (whether human or other mammalian). A preferred assay of the invention includes determining the ability of a test substance to inhibit COX-2 activity of an isolated/purified COX-2 polypeptide (including a full-length COX-2 or an active portion thereof).

In assay methods of the invention, production of product can be measured by quantifying level of substrate and/or by quantifying level of product. The greater the level of remaining substrate, the lower the level of production of the product.

In some embodiments the assay method may include determination of the selectivity of the test substance for inhibiting COX-2 as compared with another polypeptide, such as COX-1. For example, the assay method may comprise determining the inhibitory activity, e.g. IC₅₀, of the test substance against COX-1 as well as the inhibitory activity, e.g. IC₅₀ of the test substance against COX-2. Preferably, a test substance that is identified as a COX-2 selective inhibitor has 2-fold or more, such as 5 or 10-fold, greater inhibitory activity against COX-2 than COX-1. Thus, the IC₅₀ value of the test substance for inhibition of COX-2 may be 2-fold lower, preferably 5-fold or 10-fold lower than the IC₅₀ value of the same test substance for inhibition of COX-1.

Product determination may employ HPLC, UV spectrometry, radioactivity detection, or RIA (such as a commercially available RIA kit for detection of PGE). Product formation may be analysed by gas chromatography (GC) or mass spectrometry (MS), or TLC with radioactivity scanning.

In methods of the invention employing COX-2 protein, the entire (full-length) COX-2 protein sequence need not be used. Assays of the invention which test for binding between two molecules or test for COX-2 enzyme activity may use fragments or variants. Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art. Active portions of COX-2 may be used in assay methods.

An “active portion” of a COX-2 polypeptide may be used in methods of the invention. An active portion means a peptide which is less than the full length polypeptide, but which retains its essential biological activity. In particular, the active portion retains the ability to catalyse PGH₂ synthesis from arachidonic acid under suitable conditions.

mPGES-1 Assays

The present invention contemplates assay methods for determining whether a test substance, which may be a candidate compound or composition, has mPGES-1 inhibitory activity, wherein a test substance determined to have mPGES-1 inhibitory activity is identified as a substance for use in treating a breathing disorder in a mammal.

In some embodiments the assay method comprises:
contacting an mPGES-1 polypeptide with a test substance and a cyclic endoperoxide substrate of mPGES-1, under conditions in which the cyclic endoperoxide substrate of mPGES-1 would be converted by mPGES-1 into a product which is the 9-keto, 11α hydroxy form of the substrate in the absence of the test substance; and
determining the level of PGH₂ or its non-enzymatic degradations products (PGE₂, PGD₂ or PGF₂α) in the presence of the test substance compared with a control level of production of the product in the absence of the test substance,
wherein a lower level of production of the product in the presence of the test substance compared with said control level indicates that the test substance is a substance for use in treating a breathing disorder in a mammal.

The method normally comprises incubating the test substance or test substance with the enzyme and a substrate for the enzyme. The substrate may be a physiological substrate such as PGH₂, or it may be a modified or non-physiological substrate, such as a substrate designed to give rise to a detectable (e.g. coloured) product in the enzymatic reaction.

The order in which the mPGES-1 polypeptide is contacted with the test substance and with the substrate, such as PGH₂, may be varied. For example, the mPGES-1 polypeptide may be first incubated with the test substance and then contacted with substrate, or vice versa.

Thus, production of the product in the presence of the test substance may be compared with production of the product in the absence of the test substance. A lower level of product, or a lower rate of product formation indicates that the test substance inhibits the enzyme activity.

A further possibility for an assay for inhibitors is testing ability of a substance to affect PGE₂ production by a suitable cell line expressing mPGES-1 (either naturally or recombinantly). An assay according to the present invention may be performed in a cell line such as a yeast strain in which the relevant polypeptides or peptides are expressed from one or more vectors introduced into the cell.

A still further possibility for an assay is testing ability of a substance to affect PGE₂ production by an impure protein preparation including mPGES-1 (whether human or other mammalian). A preferred assay of the invention includes determining the ability of a test substance to inhibit mPGES-1 activity of an isolated/purified mPGES-1 polypeptide (including a full-length mPGES-1 or an active portion thereof).

A method of screening for a substance which inhibits activity of an mPGES-1 polypeptide (i.e. an inhibitor of mPGES-1) may include contacting one or more test substances with the polypeptide in a suitable reaction medium, testing the activity of the treated polypeptide and comparing that activity with the activity of the polypeptide in comparable reaction medium untreated with the test substance or substances. A difference in activity between the treated and untreated polypeptides is indicative of a modulating effect of the relevant test substance or substances.

The assay method may comprise:

(a) incubating an mPGES-1 polypeptide and a test compound in the presence of reduced glutathione and PGH₂ under conditions in which PGE₂ is normally produced; and
(b) determining production of PGE₂.

PGH₂ substrate for mPGES-1 may be provided by incubation of COX-2 and AA, so these may be provided in the assay medium in order to provide PGH₂.

Furthermore, mPGES-1 catalyses stereospecific formation of 9-keto, 11α hydroxy prostaglandin from the cyclic endoperoxide and so other substrates of mPGES-1 may be used in determination of mPGES-1 activity, and the effect on that activity of a test compound, by determination of production of the appropriate product.

| Substrate | Product |
|---|---|
| PGH₂ | PGE₂ |
| PGH₁ | PGE₁ |
| PGH₃ | PGE₃ |
| PGG₂ | 15(S)hydroperoxy PGE₂ |
| PGG₁ | 15(S)hydroperoxy PGE₁ |
| PGG₃ | 15(S)hydroperoxy PGE₃ |

As noted, the substrate may be any of those discussed above, or any other suitable substrate at the disposal of the skilled person. It may be PGH₂, with the product then being PGE₂.

In assay methods of the invention, production of product can be measured by quantifying level of substrate and/or by quantifying level of product. Any remaining substrate at the end of the assay or the time of terminating the assay reaction, can be converted into 12-hydroxyheptadeca trienoic acid and malon dialdehyde or PGF2α by adding iron chloride or stannous chloride, respectively. Thus, the amounts of these compounds then reflect indirectly the formation of PGE₂. Quantifying these compounds is a means of determining production of the product, by quantifying the amount of remaining substrate. The greater the level of remaining substrate, the lower the level of production of the product.

An inhibitor of mPGES-1 may be identified (or a candidate substance suspected of being a mPGES-1 inhibitor may be confirmed as such) by determination of reduced production of PGE₂ or other product (depending on the substrate used) compared with a control experiment in which the test substance is not applied. Thus, production of the product in the presence of the test substance may be compared with production of the product in the absence of the test substance. A lower level of product, or a lower rate of product formation indicates that the test substance inhibits mPGES-1 activity. Thus, the test substance may be identified as an agent for use in treating a breathing disorder in a mammal.

Product determination may employ HPLC, UV spectrometry, radioactivity detection, or RIA (such as a commercially available RIA kit for detection of PGE). Product formation may be analysed by gas chromatography (GC) or mass spectrometry (MS), or TLC with radioactivity scanning.

In methods of the invention employing mPGES-1 protein, the entire (full-length) mPGES-1 protein sequence need not be used. Assays of the invention which test for binding between two molecules or test for PGE synthase activity may use fragments or variants. Fragments may be generated and used in any suitable way known to those of skill in the art. Suitable ways of generating fragments include, but are not limited to, recombinant expression of a fragment from encoding DNA. Such fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art. Active portions of mPGES-1 may be used in assay methods.

An “active portion” of an mPGES-1 polypeptide may be used in methods of the invention. An active portion means a peptide which is less than the full length polypeptide, but which retains its essential biological activity. In particular, the active portion retains the ability to catalyse PGE₂ synthesis from PGH₂ in the presence of glutathione.

### EP3R Assays

The present invention contemplates assay methods for determining whether a test substance, which may be a candidate compound or composition, has EP3R inhibitory activity, wherein a test substance determined to have EP3R inhibitory activity is identified as a substance for use in treating a breathing disorder.

In some embodiments the method comprises:
contacting an EP3R polypeptide with a test substance and an EP3R agonist under conditions in which the EP3R agonist would activate the EP3R polypeptide in the absence of the test substance; and
determining the level of EP3R polypeptide activation in the presence of the test substance compared with a control level of EP3R polypeptide activation in the absence of the test substance,
wherein a lower level of EP3R polypeptide activation in the presence of the test substance compared with said control level indicates that the test substance is a substance for use in treating a breathing disorder in a mammal.

The EP3R agonist may be an natural agonist, such as PGE₂, or it may be a synthetic agonist. There are a number of EP3R agonists available commercially, e.g. from Biomol. One well-characterized example is Sulprostone (see: http://www.caymanchem.com/app/template/Product.vm/catalog/14765). EP3R polypeptide activation may be a conformational change in the receptor protein that results in coupling to a G-protein. EP3R polypeptide activation may be detected by monitoring an effect on adenylyl cyclase activity. For example, in a cell-based assay, activation of EP3R polypeptide present on the surface of the cell may be detected by monitoring an increase or decrease of cAMP concentration in the cell.

In some embodiments an EP3R polypeptide is present in the surface of a cell, wherein the EP3R is coupled to a reporting means. The reporting means provides an indication of receptor activation. For example, the reporting means may comprise a substance that is downstream of EP3R in an EP3R-mediated signalling pathway. By monitoring any change in the level of such a downstream substance, activation of the EP3R may be monitored. The reporting means may be monitored by any of a number of techniques including detection a fluorescent or radioactive label. In certain embodiments, the EP3R may be coupled via a G-protein to adenylyl cyclase, thereby modulating cAMP production. By monitoring cAMP levels in response to an EP3R agonist in the presence and in the absence of a test compound, the ability of the test compound to act as an antagonist of an EP3R polypeptide may be determined. Activation of human EP3R may cause a decrease in [cAMP]ᵢ and modest increases in [Ca⁺⁺]ᵢ. Therefore, an EP3R agonist may induce a decrease in intracellular [cAMP] and/or an increase in intracellular [Ca⁺⁺]. This may be monitored, for example using a FLIPR-based assay. An antagonist of EP3R may prevent or limit any EP3R agonist-induced a decrease in intracellular [cAMP] and/or an increase in intracellular [Ca⁺⁺].

### Screening In Vivo

The present invention contemplates methods for identifying a substance for use in treating a breathing disorder in a mammal. The method may employ one or more test substances that are known to inhibit or believed to inhibit the induced PGE₂ pathway.

Thus, the present invention contemplates a method for identifying a substance for use in treating a breathing disorder in a mammal, comprising:
administering a test substance to a test mammal, wherein the test substance is an inhibitor of EP3R, an inhibitor of mPGES-1 and/or a selective inhibitor of COX-2; and
determining the severity of a sign or symptom of a breathing disorder in the test mammal compared to the sign or symptom in a control mammal to which the test substance has not been administered,
wherein a lower severity of the sign or symptom of the breathing disorder in the test mammal than in the control mammal indicates that the test substance is a substance for use in treating a breathing disorder in a mammal.

For example, the test substance may be a substance that has been found to have the ability to inhibit one or more of the following:
(a) COX-2-mediated synthesis of PGH₂;
(b) mPGES-1-mediated conversion of a cyclic endoperoxide substrate of mPGES-1 into a product which is the 9-keto, 11α hydroxy form of the substrate; and
(c) EP3R agonist-mediated activation of an EP3R.

Methods for identifying a test substance that is an inhibitor of EP3R, an inhibitor of mPGES-1 inhibitor or a selective inhibitor of COX-2 are described further herein. Identifying a test substance as an inhibitor of EP3R, an inhibitor of mPGES-1 or a selective inhibitor of COX-2 may take place as an earlier stage prior to in vivo screening. In this way a plurality of compounds may be screened in vitro for the desired pharmacological activity, and those found to have the desired pharmacological activity then screened in vivo. Inhibitors of EP3R, inhibitors of mPGES-1 and selective inhibitors of COX-2 are described further herein.

The sign or symptom of a breathing disorder may include respiratory depression, apnea frequency, impaired autoresuscitation following hypoxia, decreased breathing frequency, decreased tidal volume and/or decreased gasping in response to hypoxia. Determining the severity of the sign or symptom may comprise measuring the sign or symptom following exposure of the test/control mammal to lowered oxygen tension, hypoxia and/or following administration of IL-1β, lipopolysaccharide (LPS) or PGE₂ to the test/control mammal.

As used herein, lower severity of sign or symptom of a breathing disorder means that the sign or symptom is less likely to cause harm to the mammal. For example, when the method involves determining apnea frequency following IL-1β administration, a lower frequency of apena and/or shorter apnea episodes would be considered a lower severity of the sign or symptom.

Suitable techniques for monitoring a sign or symptom of a breathing disorder are described further herein. For example, the method may employ plethysmography or impedance pneumography. The method may employ an air controlled chamber which allows for alteration of oxygen tension therein. Preferably, the chamber will be temperature controlled.

Alternatively, determining a sign or symptom of a breathing disorder may comprise monitoring brainstem respiratory activity, for example using a brainstem-spinal cord preparation isolated from the test/control mammal.

Brainstem respiratory activity may be monitored by means of an electrode as described further herein. When the method involves monitoring brainstem respiratory activity using a brainstem-spinal cord preparation isolated from the test/control mammal, the test substance may be administered prior to isolation of the brainstem-spinal cord or administered directly to the brainstem-spinal cord preparation following isolation from the test/control mammal.

The methods of the present invention may employ an ex vivo brainstem spinal cord en bloc preparation or a brainstem slice preparation. Said preparations may permit parallel monitoring of cellular, network and behavioural effects of agonists and/or antagonists, e.g. of the induced PGE₂ pathway, and environmental changes. The methods may be combined with in situ and in vivo methods as further defined herein. Induction of apnea may be achieved by environmental changes such as lowering of O₂ concentration, for example hypoxia. Alternatively or additionally, induction of apnea may be achieved by pharmaceutical or anaesthetic manipulation, such as opioid receptor agonists and/or cAMP elevating drugs, including forskolin.

The test mammal and control mammal may be rodents, and each is preferably a mouse or a rat. The method is preferably for identifying an agent for use in treating a breathing disorder in a human.

The methods of the present invention may comprise determining the severity of a sign or symptom of a breathing disorder using barometric or flow plethysmographic techniques. Such techniques may be preferred in the case of a test and control mammal being a rodent, such as a mouse or a rat. In certain embodiments, the test mammal may be a human. In such cases determining the severity of a sign or symptom of a breathing disorder may comprise using polysomnigraphic recording methods.

The test mammal and control mammal are preferably subject to identical conditions except for the absence of the test substance in the control mammal. Preferably, a control administration is given to the control mammal, such as a physiological saline solution, and is preferably administered to the control mammal by the same route as administration of the test substance to the test mammal.

In certain embodiments the test mammal and the control mammal may be the same animal. In this case determining the severity of a sign or symptom of a breathing disorder in the test mammal compared to the sign or symptom in a control mammal to which the test substance has not been administered may be performed by first determining the severity of a sign or symptom of a breathing disorder in the mammal prior to administration of the test substance (“control reading”) and secondly determining the severity of a sign or symptom of a breathing disorder in the mammal following administration of the test substance (“test reading”). The control reading and test reading may then be compared wherein a lower severity of the test reading than of the control reading indicates that the test substance is a substance for use in treating a breathing disorder in a mammal. Use of the same animal as the test mammal and control mammal may be preferred when the mammal is a human, for example in clinical study situations.

The following is presented by way of example and is not to be construed as a limitation to the scope of the claims.

### EXAMPLES

### Materials and Methods

### Animals

Neonatal mice of the inbred DBA/1lacJ strain (n=158) (Jackson Laboratory, Bar Harbor, Me.) and C57BL/6 strain (n=75) (generously provided by Dr. Beverly Koller, University of North Carolina, Chapel Hill, N.C.) were used. The microsomal prostaglandin E synthase 1 (mPGES-1) and EP3 receptor (EP3R) genes were selectively deleted in knockout mice as described previously (47, 48, both of which are expressly incorporated herein by reference). All animals were sacrificed via decapitation immediately following experimentation, and genotyping was performed using PCR and Southern blot analysis. Data from some of the wildtype DBA/1lacJ mice were included in the characterization of respiratory behavior in neonatal DBA/1lacJ mice (6). All mice were reared under standardized conditions with a 12-h light:12-h dark cycle. Food and water were provided ad libitum.

### Human Subjects

Infants (mean gestational age: 32±2 weeks) from the neonatal intensive care unit at Karolinska University Hospital were included (postnatal age mean 16±4 d) (n=12). Infants were eligible for inclusion if they underwent a lumbar puncture for clinical indications and informed written consent was obtained. These studies were performed in accordance with European Community guidelines and approved by regional ethics committees. Infants were eligible for inclusion if they underwent a lumbar puncture for clinical indications such as suspected infection, neurological changes, and cardiorespiratory problems. Infants were excluded if they had intraventricular hemorrhage (grade≧2), white matter disease (PVL-periventricluar leukomalacia), seizures, post-hemorrhagic hydrocephalus, or congenital abnormalities. Pertinent medical information was documented, including neonatal delivery data, medical conditions, infectious markers, respiratory therapy, and medications. Cardiorespiratory recordings were performed within 18 h after the lumbar puncture (mean: 4.8±1.7 h).

### Drugs

Recombinant mouse interleukin-1β (IL-1β) (Nordic Biosite AB, Täby, Sweden) was reconstituted in sterile NaCl to produce a 1 μg/ml working solution. Prostaglandin E₂ (PGE₂) (Cayman Chemicals, Ann Arbor, Mich., USA) was diluted in artificial CSF (aCSF) to a concentration of 2 nmol/μl for in vivo experiments and 20 μg/l (60 nM) for in vitro experiments.

### Unrestricted Whole-Body Flow Plethysmography

A Plexiglas chamber (35 ml) was connected to a highly sensitive direct airflow sensor (0-200 ml/min; TRN3100, Kent Scientific Corporation, Litchfield, Conn., USA). The flow signal was amplified by a four-channel amplifier (P/N 770 S/N 5; SENSElab, Somedic Sales, Hörby, Sweden), converted to digital signal, and recorded at 100 Hz by an online computer using DasyLab software (Datalog GmbH & Co. KG, Mönchengladbach, Germany). Respiratory frequency (f_{R}, breaths/min), tidal volume (V_{T}, μl/breath), and minute ventilation (V_{E}, μl/min) were calculated. Chamber temperature was maintained at 30.1±0.1° C. in accordance with the documented thermoneutral range for neonatal mice by immersing the chamber in a thermostat-controlled water bath (49). As described previously, the chamber was calibrated by repeatedly injecting standardized volumes of air (5-200 μl) with preset precision syringes (Hamilton Bonaduz AG, Switzerland) (6). 95% of gas exchange occurred within 35 s of administration, which was verified by CO₂ content analyses (Metek CD-3A and S-3A, PA, USA).

### Impedance Pneumography

Infant cardiorespiratory activity was measured non-invasively using impedance pneumography and recorded via an event monitoring system (KIDS, Hoffrichter GmbH, Schwerin, Germany). The monitor was programmed to record baseline respiratory rates as well as events exceeding the apnea threshold. Apnea was defined as a ≧10 sec reduction of the mean impedance signal amplitude during the preceding 0.5 s to less than 16% of the mean amplitude measured during the preceding 25 s. The 60 s periods before and after the event were also stored in the monitor's memory.

Plethysmography Following i.p. Injection of IL-1β or NaCl

Respiration was examined using flow plethysmography in 9 d-old DBA/1lacJ mice (n=143) and C57BL/6 mice (n=16) with variable expression of mPGES-1 and EP3R, respectively. Each mouse received an intraperitoneal injection (0.01 ml/g) of IL-1β (10 μg/kg) or vehicle. At 70 min, the mouse was placed unrestrained into the plethysmograph chamber. Respiration was assessed during 4 min of normoxia (21% O₂) followed by 1 min of hyperoxia (100% O₂). After a 5 min recovery period in normoxia, the respiratory response to anoxia (100% N₂) was examined. Finally, 100% O₂ was administered for 8 min, and the ability to autoresuscitate was evaluated. Skin temperature was recorded at baseline, at 70 min, and after removal from the chamber. Rectal temperature was not measured as rectal probe placement may alter respiratory behavior. The anogenital distance was measured to approximate gender.

### Plethysmography Following icv Injection of PGE₂ or Vehicle

Respiration was examined using flow plethysmography in 9 d-old C57BL/6 mice (n=38) with variable expression of EP3R. After the administration of sevoflurane anesthesia for approximately 60 s, PGE₂ (4 nmol in 2-4 μl aCSF) or vehicle was slowly injected into the lateral ventricle using a thin pulled glass pipette attached to polyeethylene tubing. The mouse was then placed immediately into the plethysmograph chamber. After a 10 min recovery period in normoxia, the mouse was exposed to hyperoxic and anoxic challenge as described above. Animal skin temperature was recorded at baseline and at each subsequent minute using a thermistor temperature probe.

### Brainstem Respiratory Activity

Brainstem-spinal cord preparations were rapidly isolated from 2 d-old C57BL/6 mice with EP3R^{+/+} and EP3R^{-/-} genotypes as described previously (n=11) (50, 51, both of which are expressly incorporated herein by reference). Respiratory-related activity corresponding to the inspiratory rhythm was monitored at the C4 ventral root through a glass suction electrode, recorded (5 kHz), and analyzed offline. Control recordings were performed for at least 20 min before perfusion with aCSF containing PGE₂ followed by an aCSF washout period.

Measurement of mPGES-1 Activity

Newborn mouse brains (n=33) were homogenized in 0.1M KPi (potassium inorganic phosphate) buffer containing 0.25M sucrose, 1× complete protease inhibitor (Roche Diagnostics) and 1 mM reduced glutathione followed by sonication. Membrane fraction was isolated by subcellular fractionation. mPGES-1 activity was measured in the membrane fraction as described previously (52, the disclosure of which is expressly incorporated herein by reference).

### Immunohistochemistry

Brainstems from 9 day old wildtype and EP3R-knockout pups were rapidly dissected after decapitation, fixed in 4% paraformaldehyde, and cryoprotected overnight in 15% sucrose in phosphate-buffered saline (PBS), pH 7.4. The brainstems were then rapidly frozen, and 14 μm transversal sections were serially collected in a cryostat (Leica CM3050 S, Leica Microsystems Nussloch GmbH). Sections were dried in air, rehydrated with PBS, and endogenous peroxidases were inhibited using 0.3% hydrogen peroxide for 10 min. After subsequent PBS washes, the sections were blocked and permeabilized in 5% goat serum (Jackson Immunoresearch Laboratories, West Grove, Pa.), 1% bovine serum albumin (Sigma-Aldrich), and 0.3% Triton X-100 (Sigma-Aldrich) in PBS for 45 min followed by overnight incubation with a rabbit NK-1R antibody (1:20,000 dilution; Sigma-Aldrich). The sections were then washed in PBS and incubated with a biotinylated secondary antibody (goat anti-rabbit; Vector Laboratories, Burlingame, Calif.) at a 1:50 dilution. After 1 h incubation, the sections were rinsed and incubated with peroxidase-conjugated Vectastain ABC (1:100 dilution; Vector Laboratories) for 30 min followed by Cy3-conjugated Tyramide signal amplification (TSA, 1:50; PerkinElmer, Boston, Mass.) for 2 min. The reaction was stopped in PBS and blocked with 5% donkey serum (Jackson), 1% bovine serum albumin (Sigma-Aldrich), and 0.3% Triton X-100 (Sigma-Aldrich) in PBS for 45 min. The sections were then incubated at 4C overnight with a rabbit EP3R antibody (Cayman Chemical, MI) at a 1:50 dilution. The following day, the sections were rinsed in PBS and incubated for 1 h with Alexa 488-conjugated secondary antibody (donkey anti-rabbit; Molecular probes). After following PBS washes, the sections were mounted in Vectashield Hard Set mounting medium (Vector Laboratories). To rule out the risk of possible cross-reactions, primary antibodies were titrated to determine the optimal dilutions, and control slides were included with the respective primary antibody omitted. Moreover, brainstem slices from EP3R knockout mice (n=4) were studied using the above protocols with normal NK1R staining, but no detectable EP3R. Images were processed using ImageJ software (NIH, Bethesda, Md.).

### CSF Analysis and Cardiorespiratory Recordings

Cerebrospinal fluid samples were analyzed for PGE₂ and PGE₂ metabolites using a standardized enzyme immunoassay (EIA) protocol (Cayman Chemicals, Ann Arbor, Mich., USA). Infants underwent a cardiorespiratory recording as soon as possible after the lumbar puncture (mean recording duration: 9.2±2.4 h). Blood concentrations of infectious markers (e.g., C-reactive protein, white blood cells) measured within 12 h before lumbar puncture were also recorded.

### Plethysmography Data Analysis

Periods of calm respiration without movement artefact were selected for analysis. Mean f_{R}, V_{T}, and V_{E} values during normoxia and hyperoxia as well as the anoxic response (i.e., hyperpnea, primary apnea, gasping, secondary apnea, and autoresuscitation) were analyzed as described previously (6, the disclosure of which is expressly incorporated herein by reference). Survival was recorded for all animals. Apnea was defined as cessation of breathing for three respiratory cycles. Regularity of breathing was quantified using the coefficient of variation (C.V.) (i.e., SD divided by mean of breath-by-breath interval during 60 periods).

### Infant Cardiorespiratory Data Analysis

The monitoring software was used to report baseline respiratory rates and to visualize all cardiorespiratory events. The apnea index (A.I., number apneas/h recording) was determined. The correlation between cardiorespiratory activity, infection status, and PGE₂ levels in the CSF was evaluated. All movement artifacts were excluded from analysis.

### Brainstem-Spinal Cord Preparation

The brainstem was rostrally decerebrated between the cranial nerve VI roots and the lower border of the trapezoid body so that the pons was removed. The preparation was continuously perfused in a 1.5 ml chamber with artificial cerebrospinal fluid (aCSF): 130 mM NaCl, 3.3 mM KCl, 0.8 mM KH₂PO₄, 0.8 mM CaCl₂, 1.0 mM MgCl₂, 26 mM NaHCO₃, and 30 mM D-glucose at 28° C. (flow rate, 3-4 ml/min). The solution was continuously equilibrated with 95% O₂ and 5% CO₂ to pH 7.4 (50, 51).

### Plethysmograph Data Analysis

As there is a variable response to anoxia based upon age (53), we attempted to perform all recordings at age P9; however, in an effort to minimize confounding age-related effects, weight was used as a correlate of age and only animals with weights within 1 SD of the population mean weight were included in the anoxia and survival analyses (6).

### Animal Characteristics

In the plethysmography experiments following i.p. injection of IL-1β or NaCl, the mPGES-1^{+/+} mice possessed a lower weight than mPGES-1^{-/-} mice (4.4±0.1 g vs. 4.9±0.1 g, respectively). There was no difference in animal gender. Animal skin temperature at baseline (34.7±0.1° C.) and 70 min after injection (34.8±0.1° C.) was similar between groups. After anoxia, mPGES-1^{+/+} mice possessed a higher skin temperature than mPGES-1^{-/-} mice (32.2±0.1° C. vs. 31.4±0.2° C., respectively). In the C57BL/6 mice, there was no difference in animal weight (4.5±0.1 g), animal gender, baseline temperature (34.4±0.2° C.), temperature at 70 min (34.5±0.5° C.), or after anoxia (30.4±0.1° C.). In the plethysmography experiments following icy injection of PGE₂ or vehicle, the C57BL/6 mice exhibited no difference in animal gender and post-anesthesia temperature (31.0±0.2° C.). However, EP3R^{+/+} mice weighed more than EP3R^{-/-} mice (4.9±0.1 g vs. 4.1±0.1 g, respectively). Skin temperature was measured in 9 d-old EP3R^{+/+} mice (n=13) and EP3R^{-/-} mice (n=26) at baseline and each min during normoxia, hyperoxia, and anoxia following icy injection of PGE₂ or vehicle. No difference in temperature was apparent until anoxic exposure at 23 min after injection. At that time, the EP3R^{-/-} mice possessed a lower skin temperature than EP3R^{+/+} mice (30.9±0.3° C. vs. 31.8±0.3° C., respectively). The temperature similarly differed during the post-anoxic period at 30-31 min (29.8±0.2° C. vs. 30.4±0.1° C., respectively).

### Statistics

One-way ANOVA compared those parameters with normal distribution and equal variance. Multiple comparisons were performed using the Student's t post-hoc test. Wilcoxon X² test was used for nonparametric measurements and data with non-Gaussian distributions. Change in variables over time was examined using MANOVA repeated measures design. The Spearman's Rho Correlation test determined correlations between variables. Data are presented as mean±SEM. A value of P<0.05 was considered statistically significant.

### Example 1

### Endogenous Brainstem mPGES-1 Activity and Tonic Respiratory Effect

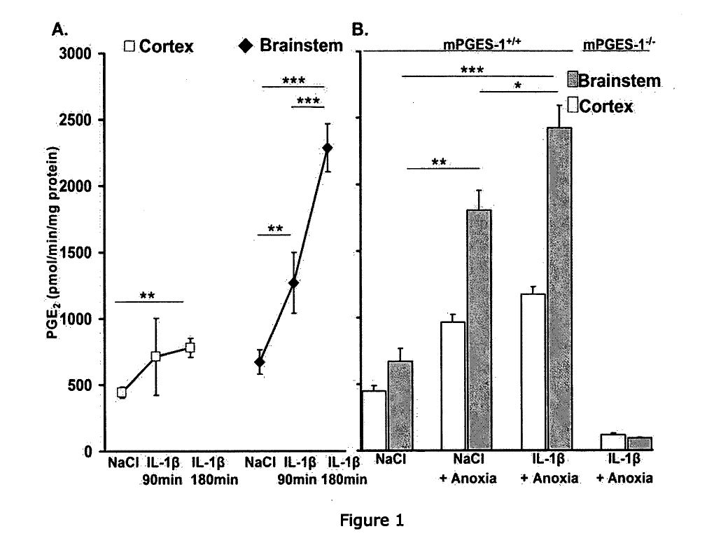

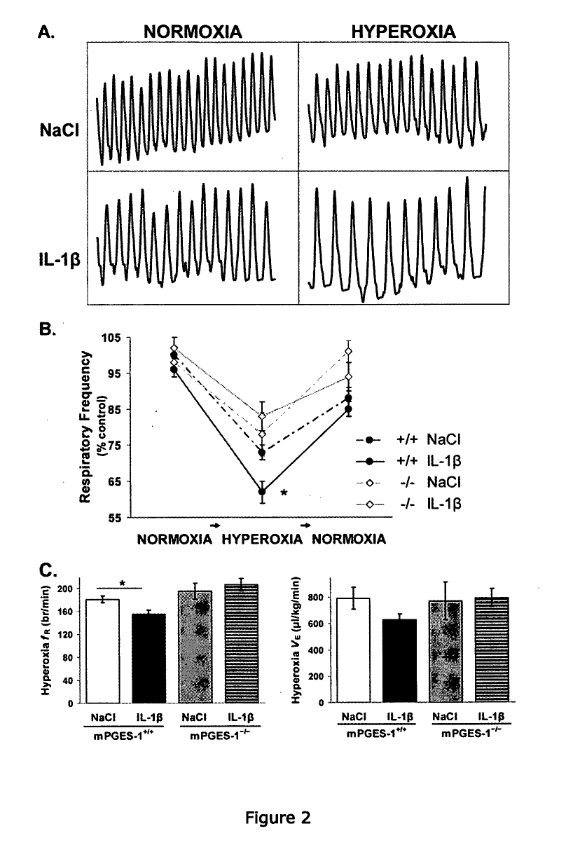

We first examined endogenous PGE₂ production and its effects on ventilation in 9 d-old mPGES-1^{+/+} and mPGES-1^{-/-} mice. Wildtype mice exhibited basal microsomal prostaglandin E synthase-1 (mPGES-1) activity that was higher in the homogenized brainstem than the homogenized cortex (FIG. 1). Breathing during normoxia was similar between genotypes, although f_{R} tended to be lower in mPGES-1^{+/+} mice than mPGES-1^{-/-} mice (Kruskal-Wallis, P=0.03; Student's t post-hoc test, P=0.18) (Table 1). The central respiratory drive was examined by a 1 min hyperoxic challenge (100% O₂, 1 min). Mice from both genotypes responded to hyperoxia with a reduction in respiratory frequency (f_{R}) (FIG. 2). However, the respiratory depression was greater in mPGES-1^{+/+} mice than mPGES-1^{-/-} mice (27±2% vs. 19±3%, respectively).

TABLE 1
Respiration during normoxia and hyperoxia in mPGES-1
mice following peripheral IL-1β administration.

| | | Normoxia | | | Hyperoxia | | |
|---|---|---|---|---|---|---|---|
| Genotype | Treatment | f_{R} | V_{T} | V_{E} | f_{R} | V_{T} | V_{E} |
| mPGES- | NaCl (n = 33) | 234 ± 6 | 3.2 ± 0.1 | 745 ± 30 | 181 ± 6 | 4.4 ± 0.4 | 791 ± 85 |
| 1^{+/+} | IL-1β (n = 33) | 224 ± 5 ^{#} | 3.2 ± 0.2 | 730 ± 38 | 155 ± 7 * | 3.9 ± 0.2 | 628 ± 43 |
| mPGES- | NaCl (n = 15) | 247 ± 7 | 2.8 ± 0.2 | 684 ± 45 | 195 ± 14 | 3.9 ± 0.5 | 771 ± 142 |
| 1^{-/-} | IL-1β (n = 19) | 245 ± 7 | 2.7 ± 0.1 | 660 ± 41 | 206 ± 11 | 3.9 ± 0.3 | 795 ± 67 |

Respiratory frequency (f_{R}, breaths/min), tidal volume (V_{T}, μl/br/g), and minute ventilation (V_{E}, μl/min/g) during normoxia and hyperoxia (100% O₂) were examined in 9 d-old mPGES-1^{+/+} and mPGES-1^{-/-} mice after intraperitoneal injection of IL-1β or vehicle. When comparing treatment effects within each genotype, IL-1β tended to reduce basal f_{R} in mPGES-1^{+/+} mice (Wilcoxon X², P=0.17), but not in mPGES-1^{-/-} mice. All mice responded to hyperoxia with a reduction in f_{R}. IL-1β depressed f_{R} during hyperoxia in mPGES-1^{+/+} mice, and this effect was not apparent in mPGES-1^{-/-} mice. mPGES-1^{+/+} mice exhibited a greater extent of respiratory depression during hyperoxia compared to mPGES-1^{-/-} mice. Data are presented as mean±SEM. * P<0.05. *P<0.05 when normalized by weight.

The present results demonstrate an endogenous expression of mPGES-1 activity, particularly in the brainstem. mPGES-1 is expressed mainly by endothelial cells along the blood-brain barrier (BBB) (25). A constitutive as well as rapidly inducible expression of mPGES-1 at endothelial cells overlying the brainstem, near crucial respiration-related centers, suggests an important role of PGE₂ in control of breathing. The significant respiratory depression in wildtype mice compared to mice lacking mPGES-1 during hyperoxia also provides evidence that endogenous PGE₂ has a tonic effect on respiratory rhythmogenesis during the perinatal period.

Previous studies have reported that prostaglandin synthesis inhibitors, which block endogenous prostaglandin production, increase fetal breathing movements as well as central respiration during early postnatal life (26-28). Developmental changes occur in the modulatory effects of prostaglandin with an initial inhibition of ventilation during the perinatal period (18, 26, 27, 29) followed by smaller changes in respiration with increasing age (19). However, PGE₂ may still disrupt regular breathing with induction of apnea at older ages (19). Developmental changes could be secondary to alterations in brainstem PGE₂ receptor expression beyond the perinatal period, although EP3R gene and protein are expressed in adult rodent RVLM (20, 21, 30). In addition, even though prostaglandin binding density may decrease, it is located in the same brainstem regions at all ages (31). Further investigation of the ontogenesis of EP3R expression and mechanisms underlying potential developmental changes in the respiratory effects of PGE₂—e.g., post-translational EP3R modification, suprapontine influences—is warranted.

### Example 2

### IL-18 and Anoxia Induced mPGES-1 Activity in the Mouse Brainstem

We also measured the effect of IL-1β and short anoxic exposure (100% N₂, 5 min) on mPGES-1 activity in the homogenized brainstem and cortex of 9-d old mPGES-1^{+/+}, mPGES-1^{-/-}, and EP3R^{+/+} mice (FIG. 1). IL-1β induced a time-dependent increase in mPGES-1 activity, particularly in the brainstem. Specifically, there was a two- and four-fold increase in brainstem mPGES-1 activity at 90 and 180 min, respectively, after IL-1β administration, whereas cortex activity remained unchanged between 90 and 180 min. Anoxic exposure also induced mPGES-1 activity in both brainstem and cortex. Notably, there was an additive effect of IL-β and short anoxic exposure on mPGES-1 activity, which was more pronounced in the brainstem. EP3R wildtype mice displayed similar mPGES-1 activity compared to the mPGES-1 wildtype mice at 90 min after IL-1β. Moreover, the EP3R mice also had higher mPGES-1 activity in the brainstem than the cortex (PGE₂: 1111±49 and 710±44pmol/min/mg protein, respectively).

PGE₂ also appears to play a crucial role in the respiratory response to anoxia. A short anoxic exposure increased mPGES-1 activity in the homogenized mouse brain. This rapid increase in mPGES-1 activity in vivo is a new finding. Previous studies have shown that anoxia induces PGE₂ production in mice cortical sections ex vivo and prostaglandin H synthase-2 mRNA expression in the piglet brain (32, 33). Transient asphyxia similarly increases PGE₂ concentrations in the newborn guinea pig brain, and this effect is inhibited by pretreatment with indomethacin (34).

No known mechanisms of mPGES-1 enzyme regulation may explain the rapid changes in mPGES-1 activity revealed here. Induced gene expression is unlikely to occur during such a short anoxic event. However, post-transcriptional regulation of constitutively expressed mPGES-1, e.g., phosphorylation, is a potential etiology. Stabilization of mPGES-1 mRNA is another possibility, as previously shown with COX-2 mRNA in a human cell system (35) and recently in cardiac myocytes (36). Further investigation is required to clarify the underlying mechanism.

### Example 3

### IL-1β Depressed Respiration in mPGES-1^{+/+} Mice, but not in mPGES-1^{-/-} or EP3R^{-/-} Mice

In order to examine the role of PGE₂ in mediating the ventilatory effects of IL-1β, we analyzed respiration during normoxia and hyperoxia (100% O₂, 1 min) using flow plethysmography after i.p. administration of IL-1β or vehicle in 9 d-old mPGES-1^{+/+}, mPGES-1^{-/-}, and EP3R^{-/-} mice (FIG. 2, Table 1). All mice, irrespective of treatment, responded to hyperoxic challenge with a reduction in f_{R}, but IL-1β-treated wildtype mice exhibited a greater respiratory depression than vehicle-treated wildtype mice. IL-1β also tended to reduce basal f_{R} in mPGES-1^{+/+} mice (Kruskal-Wallis, P=0.03; Student's t post-hoc test, P=0.17). Conversely, IL-1β did not alter ventilation during normoxia or hyperoxia in mPGES-1^{-/-} or EP3R^{-/-} mice.

The present results indicate that mPGES-1 activation is necessary for IL-1β to depress central respiration. First, IL-1β increased brainstem mPGES-1 activity in a time-dependent manner. Second, IL-1β depressed respiration in mPGES-1^{+/+} mice, but not in mPGES-1^{-/-} mice. Indomethacin, by blocking prostaglandin synthesis, has been shown to similarly attenuate the effects of IL-1β on basal respiration (5).

### Example 4

### IL-1β Worsened Anoxic Survival in Wildtype Mice, but not Mice Lacking mPGES-1 or EP3R

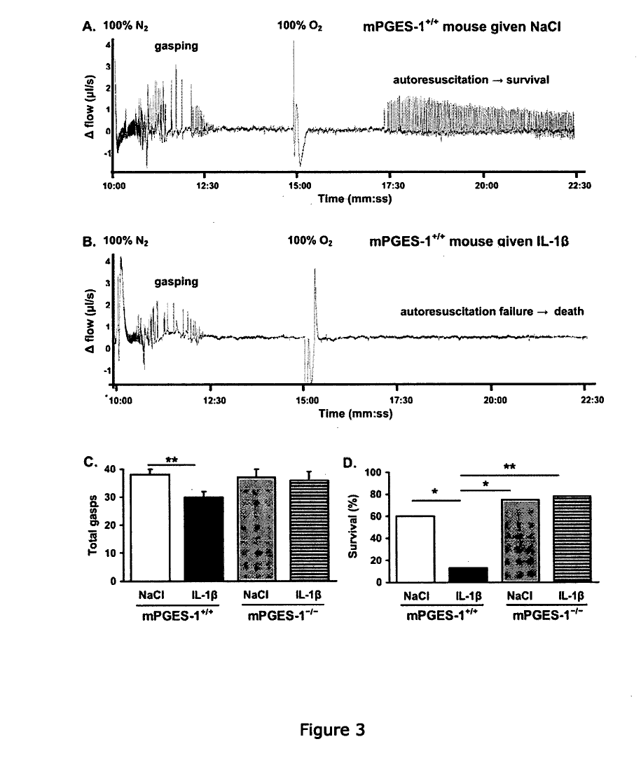

Next, we investigated whether IL-18 affects the hypoxic ventilatory response and autoresuscitation following hypoxic apnea via a PGE₂-mediated mechanism. Using flow plethysmography, respiration during anoxia (100% N₂, 5 min) followed by hyperoxia (100% O₂, 8 min) was examined beginning at 80 min after i.p. injection of IL-1β or vehicle in mPGES-1^{+/+}, mPGES-1^{-/-}, and EP3R^{-/-} mice (FIG. 3, Table 2). All mice exhibited a biphasic response to anoxia with an initial increase in ventilation (i.e., hyperpnea) followed by a hypoxic ventilatory depression (i.e., primary apnea, gasping, secondary apnea). IL-1β reduced the number of gasps in mPGES-1^{+/+} mice, but not in mPGES-1^{-/-} mice. IL-18-treated mPGES-1^{+/+} mice also tended to have a shorter gasping duration compared to IL-1β-treated mPGES-1^{-/-} mice (Kruskal-Wallis, P=0.19; Student's t post-hoc test, P=0.003). Fewer gasps and a shorter gasping duration were correlated with decreased anoxic survival. IL-1β significantly reduced anoxic survival in mPGES-1^{+/+} mice, but did not decrease survival in mice lacking the mPGES-1 or EP3R genes. IL-1β was unable to affect the hypoxic ventilatory response of EP3R^{-/-} mice.

TABLE 2
Biphasic ventilatory response to anoxia.

| | | Hyperpnea | | Gasping Response | | |
|---|---|---|---|---|---|---|
| Genotype | Treatment | f_{R} | Duration | Gasp # | Gasp f_{R} | Duration |
| mPGES- | NaCl (n = 20) | 368 ± 11 | 63 ± 2 | 38 ± 2 | 25 ± 1 | 94 ± 6 |
| 1^{+/+} | IL-1β (n = 17) | 390 ± 11 | 61 ± 2 | 30 ± 2 ** | 23 ± 1 | 82 ± 7 |
| mPGES- | NaCl (n = 8) | 339 ± 25 | 55 ± 4 | 37 ± 3 | 23 ± 3 | 113 ± 18 |
| 1^{-/-} | IL-1β (n = 12) | 338 ± 24 | 57 ± 2 | 36 ± 3 | 18 ± 2 | 146 ± 23 |

Newborn mice with variable expression of microsomal prostaglandin E synthase-1 (mPGES-1) were exposed to anoxia at 80 min after peripheral administration of IL-1β or vehicle. Mice exhibited an initial increase in f_{R}, V_{T}, and V_{E} during hyperpnea followed by gasping response during hypoxic ventilatory depression. When comparing treatment effects within each genotype, IL-1β decreased the number of gasps in wildtype mice, whereas this effect was not observed in mice with reduced expression of mPGES-1. Data are presented as mean±S.E.M. ** P<0.01.

This study demonstrates that PGE₂ also plays a crucial role in mediating the anoxic ventilatory effects of IL-1β. IL-1β inhibited autoresuscitation following hypoxic apnea in wildtype mice, but not in mice lacking mPGES-1 or EP3R. Previous studies have shown that indomethacin attenuates the adverse effects of IL-1β on hypoxic gasping and anoxic survival in neonatal rats (5).

### Example 5

### PGE₂ Decreased Brainstem Respiration-Related Activity and Induced Apnea Via EP3R

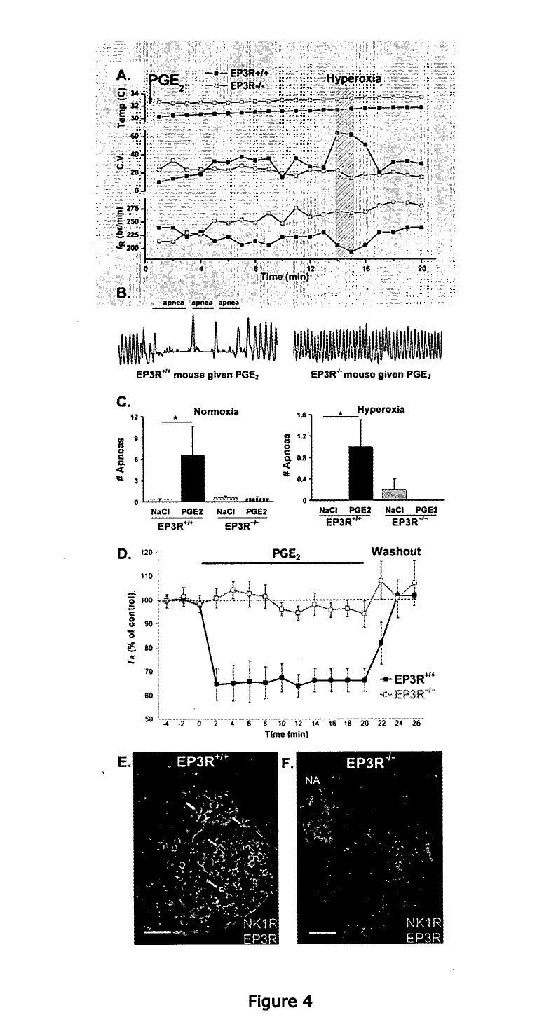

In order to better determine whether PGE₂ depresses respiration by binding specifically to brainstem EP3 receptors, central respiratory activity was measured using the en bloc brainstem-spinal cord preparation of 2-3 d-old EP3R^{+/+} and EP3R^{-/-} mice following administration of artificial cerebrospinal fluid or PGE₂. During control conditions, similar respiratory activity was recorded in preparations from EP3R^{+/+} and EP3R^{-/-} mice. However, PGE₂ reversibly inhibited respiration-related frequency in EP3R^{+/+} preparations, but had no affect on EP3R^{-/-} preparations (FIG. 4).

The ability of PGE₂ to alter breathing via EP3R was further assessed using flow plethysmography. Following icy injection of PGE₂ or vehicle in EP3R^{+/+} and EP3R^{-/-} mice, respiration during normoxia and hyperoxia was analyzed (FIG. 4 and Table 3). PGE₂ induced a significantly greater apnea frequency and irregular breathing pattern during normoxia and hyperoxia in EP3R^{+/+} mice, but not in not EP3R^{-/-} mice. The mice were subsequently exposed to anoxia followed by hyperoxia, which enabled them to autoresuscitate. All mice continued gasping beyond the 5 min anoxic exposure, and only one of 38 mice failed to autoresuscitate (PGE₂-treated EP3R^{-/-} mouse). PGE₂ did not alter the gasping response or anoxic survival of EP3R^{+/+} or EP3R^{-/-} mice compared to vehicle. Finally, we investigated whether respiration-related neurons in the rostral ventrolateral medulla (RVLM) express EP3R. Specifically, NK1R immunolabeling was used as a tool to identify respiration-related neurons located in the RVLM ventral to the nucleus ambiguous and including the pre-Bötzinger Complex (22-24). We show that these neurons co-expressed NK1R and EP3R (FIG. 4).

TABLE 3
Respiration during normoxia, hyperoxia, and anoxia in EP3R mice following central PGE2 administration.

| | | Normoxia | | | Hyperoxia | | | Hyperpnea |
|---|---|---|---|---|---|---|---|---|
| Genotype | Treatment | f_{R} | V_{T} | V_{E} | f_{R} | V_{T} | V_{E} | f_{R} |
| EP3R^{+/+} | NaCl (n = 7) | 281 ± 17 | 3.8 ± 0.4 | 1065 ± 75 | 234 ± 19 | 7.0 ± 3.0 | 1598 ± 642 | 327 ± 13 |
| | PGE₂ (n = 6) | 247 ± 13 * | 3.7 ± 0.4 | 901 ± 154 | 190 ± 16 | 4.4 ± 1.1 | 745 ± 102 | 267 ± 11 ** |
| EP3R^{-/-} | NaCl (n = 12) | 247 ± 15 | 5.3 ± 0.6 | 1322 ± 157 | 200 ± 23 | 5.4 ± 0.9 | 1057 ± 213 | 288 ± 11 |
| | PGE₂ (n = 13) | 256 ± 10 | 5.2 ± 0.5 | 1350 ± 129 | 229 ± 9 | 6.7 ± 1.3 | 1509 ± 299 | 290 ± 9 |

Respiratory frequency (f_{R}, breaths/min), tidal volume (V_{T}, μl/br/g), and minute ventilation (V_{E}, μl/min/g) during normoxia, hyperoxia (100% O₂), and anoxia (100% N₂) were examined in 9 d-old EP3R^{+/+} mice (n=13) and EP3R^{-/-} mice (n=25) after intracerebroventricular (icy) injection of PGE₂ or vehicle. When comparing treatment effects within each genotype, PGE₂ significantly depressed f_{R} during normoxia and hyperpnea in EP3R^{+/+} mice, but not in EP3R^{-/-} mice. PGE₂ also tended to reduce f_{R} during hyperoxia in EP3R^{+/+} mice (ANOVA, P=0.11), but not in EP3R^{-/-} mice. Data are presented as mean±SEM. * P<0.05, ** P<0.01.

The results presented in the preceding examples provide evidence that after mPGES-1 activation, newly synthesized PGE₂ exerts the respiratory actions of IL-1β centrally. We show here that PGE₂ hindered breathing in wildtype mice, consistent with studies demonstrating that PGE₂ depresses respiration in fetal and newborn animals (18, 29, 37). Moreover, these effects occur centrally since PGE₂ did not alter peripheral chemosensitivity in vivo and directly inhibited brainstem respiratory activity in vitro. Previous studies have shown that PGE₂ inhibits respiration-related neurons in neonatal rats (5) and similarly inhibits fetal breathing movements in sheep following sham-operation or denervation of the carotid sinus and vagus nerve (38).

Furthermore, the modulatory effects of PGE₂ occur via binding to brainstem EP3 receptors. IL-18 was unable to alter respiration in EP3R^{-/-} mice. PGE₂ induced apnea and irregular breathing in vivo in EP3R^{+/+} mice, but not in EP3R^{-/-} mice. Finally, the presence of EP3 receptors was required to inhibit brainstem respiration-related rhythmic activity in vitro. While the specific prostaglandin receptor subtype EP3R has been localized to the NTS and RVLM (20, 21), no prior studies have shown that the respiratory effects of prostaglandin occur via action at these receptors and that they are expressed in respiration related neurons.

The results of the preceding examples suggest that PGE₂ induced by IL-1β as well as hypoxia selectively modulates respiration-related neurons in the RVLM, including the pre-Bötzinger complex (preBötC), via EP3R. Other neuromodulators, including PGE₁, have been shown to inhibit preBötC neurons and slow respiration-related rhythm (22, 23), and preBötC lesions may disrupt anoxic gasping and evoke central apneas and ataxic breathing (39, 40). Moreover, these respiration-related neurons were recently shown to be critical for adequate response to hypoxia, maintaining brainstem homeostasis with gasping and autorescuscitation and thus restoring oxygen levels (41). PGE₂-induced depression of this vital brainstem neuronal network, e.g., during an infectious response, could result in gasping and autoresuscitation failure and ultimately death.

### Example 6

### Central PGE₂ Concentration Correlated with Increased Apnea Frequency in Human Infants

In order to further elucidate the mechanism underlying the association between infection and apnea in human newborns, we examined the association between the infectious marker C-reactive protein (CRP), cerebrospinal fluid PGE₂ levels, and apnea events in newborn infants. CRP was positively correlated with central PGE₂, and there was a positive association between PGE₂ concentrations in the CSF and apnea frequency (FIG. 5).

Apnea is a common presenting sign of sepsis in the neonatal population (1), yet the mechanism underlying this association remains unclear. Here, we show that the infectious marker CRP is correlated with elevated PGE₂ levels in the CSF of human neonates. Importantly, we also demonstrate that PGE₂ is associated with an increased apnea frequency. These findings suggest that infection depresses respiration in human neonates via systemic release of cytokines followed by the biosynthesis and central action of PGE₂. The mechanism described here could explain previous reports showing an independent association between CRP levels and the apnea/hypopnea index in children with sleep apnea (42) as well as a positive correlation between IL-1β concentrations in pharyngeal secretions of human infants and clinical severity of apnea (8). Transient apneas are also a common side effect of prostaglandin treatment in human neonates (43), which may be due to activation of EP3 receptors in brainstem respiration-related centers. Furthermore, our data provide an explanation for the positive correlation between central apneas and urine PGE metabolites in newborn infants (44).

Inflammatory mediators have been proposed as important markers for detecting infection and asphyxia in newborns. The rapid synthesis of PGE₂ in response to cytokine and hypoxic stimulation may make it particularly useful in the diagnosis and surveillance of infants with increased apneas due to suspected infection or asphyxia. Studies to evaluate the potential diagnostic benefits of monitoring PGE₂ compared to other infectious markers such as CRP are necessary.

The present results have important treatment implications for neonatal apnea related to infection since the adverse effects of IL-1β were attenuated by selectively deleting the mPGES-1 and EP3R genes. Indomethacin has been used previously to treat apnea of prematurity (45). However, indomethacin causes multiple adverse effects in the newborn population (46), and thus treatment modalities selectively targeting mPGES-1 or EP3 receptors could be more beneficial.

The foregoing examples demonstrate that systemic interleukin-1β depresses breathing and autoresuscitation via mPGES-1 activation and PGE₂ binding to EP3 receptors in respiration-related regions of the brainstem (FIG. 6). Additionally, severe hypoxia rapidly induces mPGES-1 activity, indicating that endogenous PGE₂ may modulate brainstem respiratory neurons during hypoxia in the newborn period. Lastly, a correlation is revealed between infection, central PGE₂, and apnea events in human neonates.

### Example 7

### PGE₂-Metabolite Correlation to Degree of Birth Asphyxia and HIE

The present inventors investigated the hypothesis that perinatal asphyxia in human infants causes rapid release of PGE2 and neurological damage.

### Patients

Sixty three term infants (>37 wk gestation) treated at Karolinska Hospital in Stockholm were enrolled in the study after parental consent, between October 1999 and September 2004. Forty three infants fulfilled the following criteria for birth asphyxia: 1) Signs of fetal distress as indicated by cardiotocographic pattern of late decelerations, absent variability or bradycardia, meconium staining of amniotic fluid, scalp pH<7.2 or Laktat>4.8 mmol/; 2) Postnatal stress as indicated by Apgar score <6 at 5 minutes and need for neonatal resuscitation in the delivery room for >3 minutes or pH<7.1, BE<−15 (or Laktat>4.8 mm/L) in cord blood or venous blood from the patient taken within 60 min from birth; 3) Neurological signs of encephalopathy within 6 hours of birth.

Exclusion criteria were congenital malformations, chromosomal abnormalities and encephalopathy unrelated to asphyxia; metabolic diseases, intrauterine/perinatal infections with confirmed meningitis.

The control group consisted of 20 infants with suspected infection but negative bacterial and viral cultures from blood and CSF, no leucocytes and normal amounts of proteins in CSF, and no findings suggesting CNS pathology.

### Clinical Assessment

Neurological assessment (95, the disclosure of which is expressly incorporated herein by reference) was done on the first few hours before enrolling the patient into the study, then at approximately 12, 36 and 72 hours after birth and on day 7 on patients in the neonatal intensive care. Hypoxic ischemic encephalopathy (“HIE”) was classified as mild, moderate or severe according to the criteria of Sarnat and Sarnat (96, the disclosure of which is expressly incorporated herein by reference). Continuous amplitude-integrated EEG was used to assess all patients for the first days of life. On all patients with moderate and severe HIE a CT- or MRI scan of the brain was done on the third day of life and EEG registration in the first week.

Neurological assessment of surviving patients was done at 3, 6 and 18 months of age by a neuropediatrician. Based on the outcome children were classified as (1) normal outcome, (2) mild motor impairment; mild symptoms of abnormal muscular tone or delayed motor development, or (3) adverse outcome; cerebral palsy (diplegia, hemiplegia, tetrplegia), mental retardation, seizures or death.

### Apgar Score

The Apgar score is a practical method of evaluating the physical condition of a newborn infant shortly after delivery. The Apgar score is a number arrived at by scoring the heart rate, respiratory effort, muscle tone, skin colour, and response to stimulation (e.g. a catheter in the nostril or rubbing the sole of the foot). Each of these objective signs can receive 0, 1, or 2 points. A perfect Apgar score of 10 means an infant is in the best possible condition. An infant with an Apgar score of 0-3 needs immediate resuscitation.

The Apgar score is done routinely 60 seconds after the birth of the infant (APGAR-1 min) and then it is commonly repeated 5 minutes after birth (APGAR-5 min). In the event of a difficult resuscitation, the Apgar score may be done again at 10, 15, and 20 minutes. An Apgar score of 0-3 at 20 minutes of age is predictive of high morbidity (disease) and mortality (death).

### CSF Sampling

CSF spinal tabs were performed on the first 24 hours (13.9+/−5.8) after birth and/or between 30 and 80 hours (57.8+/−9.9). Each spinal tab collected amount of 1-2 ml of CSF. The samples were spun at 3000 rpm at 4 degrees for 10 minutes and the supernatant stored at −80 degrees C. in aliquots of 0.5 ml until analyzed.

### PGE₂ Assays

PGE₂ and PGE₂ metabolites were analyzed in Cerebrospinal fluid samples using a standardized enzyme immunoassay (EIA) protocol (Cayman Chemicals, Ann Arbor, Mich., USA).

### Protein Analysis (BCA Assay)

BCA assay was done to determine protein levels in the samples.

### Statistical Analysis

Clinical data are presented as medians and interquartile ranges for descriptive purposes unless stated otherwise. Mann-Whitney test was applied to analyze differences between patients and controls. Kruskal-Wallis test was used to determine the association between PGE2-metabolite or cytokine level and degree of HIE or clinical outcome.

### Results

The patient group (n=43) was divided into three subgroups according to Sarnat and Sarnat classification of HIE. Thirteen infants had according to this classification mild HIE (HIE I) and all of them had normal outcome. Sixteen infants had moderate HIE (HIE II), eight of those infants had adverse neurological outcome with cerebral palsy, psychomotor retardation and seizure problems, additionally two infants had mild motor impairment and six had normal outcome. Fourteen infants had severe HIE (HIE III), eight of them died on first to 12^{th} day of life and 6 patients survived with adverse neurological outcome; spastic tetraplegic cerebral paresis, psychomotor retardation, microcephali and complex seizures.

Clinical data for patients and control groups are given in table 4 below. No differences were found between patients and controls regarding gestational age and birth weight, but there was a difference regarding 5 minutes Apgar score as well as umbilical artery or early patient pH (p<0.001). The Apgar score was obtained in response to stimulation (such as inserting a catheter into the infant's nose or rubbing the sole of the infant's foot). No difference was found between patient groups for any of the clinical data. Level of CRP in blood was non-significant for both controls and patients.

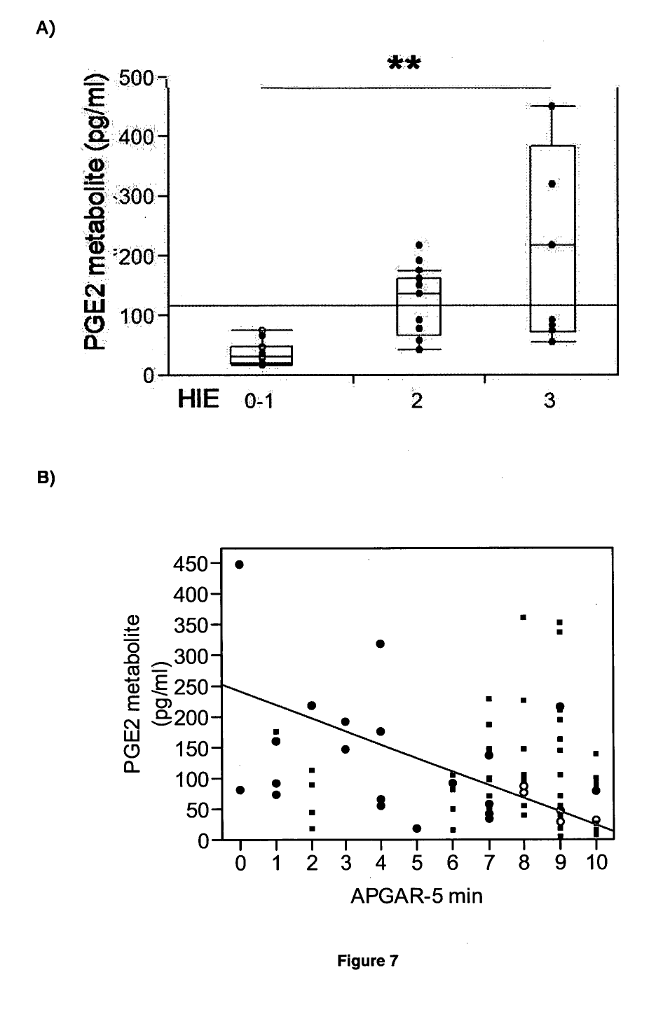

As shown in FIG. 7A, the degree of birth asphyxia (APGAR score at 5 and 10 min) as well as neurological outcome correlate to CSF PGE₂-metabolite levels in full term infants.

Similarly, as shown in FIG. 7B, the PGE2-metabolite also correlates to APGAR score at 5 minutes after birth, an indicator for the condition of the newborn child, and likely the degree of asphyxia during birth.

These results suggest that PGE₂ is rapidly released during severe hypoxia (asphyxia) in human infants and may, therefore, be used as a diagnostic tool and/or a target for therapeutic intervention in newborn asphyxiated babies.

TABLE 4
Clinical data of study cohort.

| | Controls | HIE-1 | HIE-2 | HIE-3 |
|---|---|---|---|---|
| Number of patients | 20 | 13 | 16 | 14 |
| Gestational age (wk) ¹ | 38.9 (38.2-41.1) | 41.2 (38.7-41.9) | 40.4 (38.9-41.1) | 39.3 (39.0-40.6) |
| Birth weight (g) ¹ | 3609 (3459-4004) | 3400 (3225-4150) | 3550 (3274-3975) | 3500 (3250-3600) |
| 5 min Apgar score ² | 10 (7-10) | 5 (1-7) | 4 (2-7) | 4 (0-7) |
| Arterial pH ¹ | 7.3 (7.25-7.35) | 7.01 (6.9-7.1) | 6.86 (6.69-6.98) | 6.82 (6.66-7.07) |
| Early CSF samples (LP1) ³ | 10 | 10 | 9 | 10 |
| Late CSF samples (LP2) ³ | 10 | 5 | 14 | 8 |
| Maternal infection | 0 | 2 | 2 | 2 |
| Outcome: | | | | |
| Normal | 20 | 13 | 6 | 0 |
| Adverse ⁴ | 0 | 0 | 10 | 6 |
| Death | 0 | 0 | 0 | 8 |

¹ Median (p25-p75),
² Median (Range),
³ Mean +/− SD,
⁴ other than death

### Example 8

### Urinary Prostaglandin Metabolites, Inflammation and Correlation to Respiratory Dysfunction

The present inventors have developed a sensitive and specific method for detection of urinary Prostaglandin E metabolites (u-PGEM) using a protocol for Triple quadrople Mass spectrometry-tetranor PGEM.

Validation studies indicate that the triple quadrople mass spectrometry-tetranor PGEM method exhibits <5% interexperimental variation between samples taken from same subject. Urine samples stored at room temperature were found to degrade PGE metabolites with a t_{1/2} estimated at approximately 2 hours. In contrast, direct storage at 4° C. significantly reduced sample degradation. Samples stored between −20° C. and -80° C. exhibited virtually no apparent degradation of PGE metabolites when comparing samples.

### Sample Preparation

Urine sample were acidified to ˜pH 3.0 by adding 2% (v/v) 1 M citric acid. An aliquot of 145 μl acidified urine was then spiked with 5 μl internal standard solution containing 9 pmol/μl tetranor PGEM-d6 and 0.45 pmol/μl 11β-PGF2α-d4 in ethanol. 100 μl were injected to the LC-MS/MS instrument. Samples for standard curves and quality controls were prepared in PBS acidified with 2% (v/v) 1 M citric acid. An aliquot of 140 μl acidified PBS was then spiked with 5 μl internal standard solution (as above) and 5 μl standard solution (30 to 900 pmol/μl tetranor PGEM and 3 to 90 pmol/μl 11β-PGF2α). 100 μl were injected to the LC-MS/MS instrument to obtain a standard curve from 100 to 3000 pmol tetranor PGEM and 10 to 300 pmol 11β-PGF2α.

LC-MS/MS conditions: The analytes were separated on a Phenomenex Synergi Hydro RP column (100 mm×2 mm i. d., 2.5 μm particle size and 100 Å pore size) using H₂O with 0.0005% FA and ACN with 0.0005% FA as mobile phase. Directly after injection of the sample a linear gradient from 15 to 60% ACN, 0.0005% FA was applied over 15 min, followed by washing with 95% ACN, 0.0005% FA and re equilibration. Total run time was 21 min. The mass spectrometer was operated in negative ion mode with an electrospray voltage of −3000 V at 350° C. For detection and quantification of prostaglandin metabolites multiple reaction monitoring (MRM) was used, recording the transition 327.1>255.3 for tetranor PGEM as well as 333.1>263.3 for tetranor PGEM-d6 (fragmentor energy 70 V, collision energy -20 V, dwell time 100 msec) and 353.3>309.3 for 11β-PGF2α; -PGF2α; as well as 357.3>313.3 for 11β-PGF2α-d4 (fragmentor energy 150 V, collision energy −15 V, dwell time 100 msec). All quadrupoles were working at unit resolution to obtain highest sensitivity.

The results described herein show that elevated levels of u-PGEM obtained from adults, children (1-16 years) and infants (0-1 year) provide a reliable indication of inflammation and are significantly associated with respiratory dysfunction (including apnea).

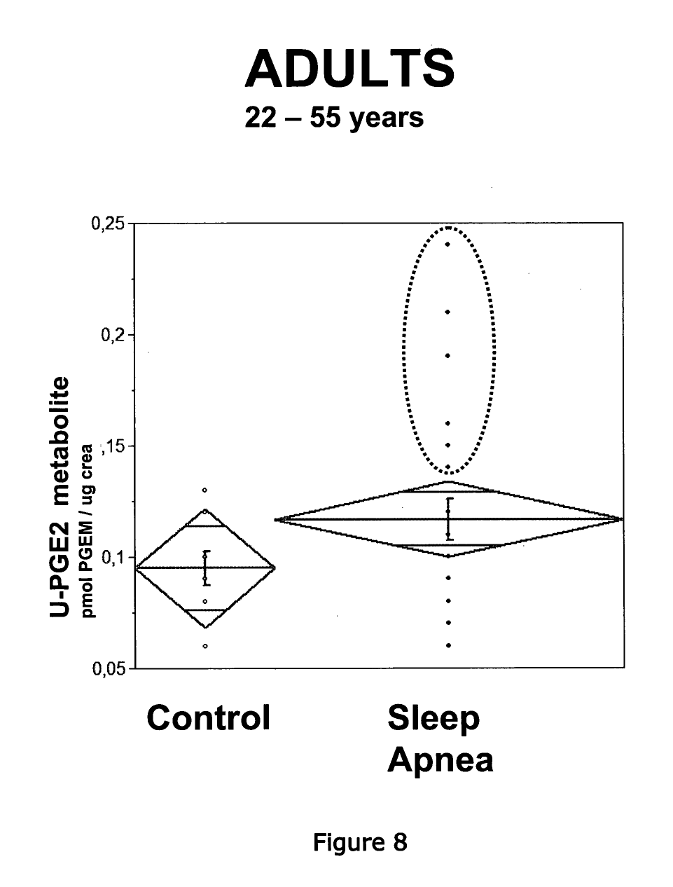

Urine samples from healthy adult controls (n=10) were compared with urine samples obtained from patients with “obstructive” sleep apnea syndrome (OSAS) (n=24; age 22-55 years). Sleep-related apnea syndrome (“Obstructive sleep apnea syndrome” (OSAS)-snorers) amount to around 3% of females and 5% of male adult population. The results are shown in FIG. 8, in which the y-axis shows urinary PGE metabolites in units of picomol PGEM/μg creatinine. All patients with the diagnosis of obstructive sleep apnea syndrome performed a night-time sleep polysomnographic recording Laboratory test including urinary samples obtained in the morning after the sleep polysomnographic (including respiratory and saturation) recording.

The group having sleep apnea (snorers) exhibits substantially greater diversity of u-PGEM levels in comparison with the control group (note the larger spread of values). The inventors have noted a clear tendency for elevated u-PGEM levels to correlate with apneic index, i.e. number of apneas/hour. Furthermore, the patients with severe OSAS have a significant correlation between apneic index and CRP (an indirect marker of inflammation and PGE₂).

Approximately one in three adults with sleep apnea have elevated u-PGEM which correlate to the severity of apnea. Comparison between groups shown in FIG. 8 indicated p=0.12. However, when including only those with severe apneic problems and excluding those with obstructive problems (BMI value>overweight), a significant association is seen between apnea and u-PGEM levels.

The present inventors have found that individuals with high apneic index are over-represented in elevated u-PGEM subjects (i.e. those with greater than control level—see dotted ellipse of FIG. 8).

The present inventors also investigated u-PGEM levels in Prader-Willi Syndrome (PWS)_children (3-16 years of age).

Patients with Prader-Willi syndrome, (deletions of 15q11-q13) have a disturbed respiratory and cardiovascular control system with apneas especially during sleep (115). Death due to cardiorespiratory disturbances usually occurs during sleep and even if a causative factor is not established minor infectious episodes are associated in 2 out of 3 deaths (107).

We hypothesize that activation of the mPGES-1 pathway is involved in the potentially fatal exaggerated respiratory disturbances that occur during infection (see also Nature Medicine 2007, Vol. 13, No. 7, p. 789, Research Highlights: “Baby's breath”).

Known infectious and inflammatory markers hs-CRP, CRP, WBC and cytokines (IL-1β) as well as urinary-metabolites of PGE₂ are examined in parallel with cardiovascular registration. This is performed infants and adults with Prader Willi Syndrome 1) during regular yearly physical examination and 2) 24 hours after signs of infection (Temperature >38.5° C.) and 3) at least one week after clinical infection has subsided. Analyses are performed at the regular clinical laboratories and at the research laboratories at the Karolinska core proteomic facilities using the triple quadrupole mass spectrometer for quantification of known metabolites and peptides.

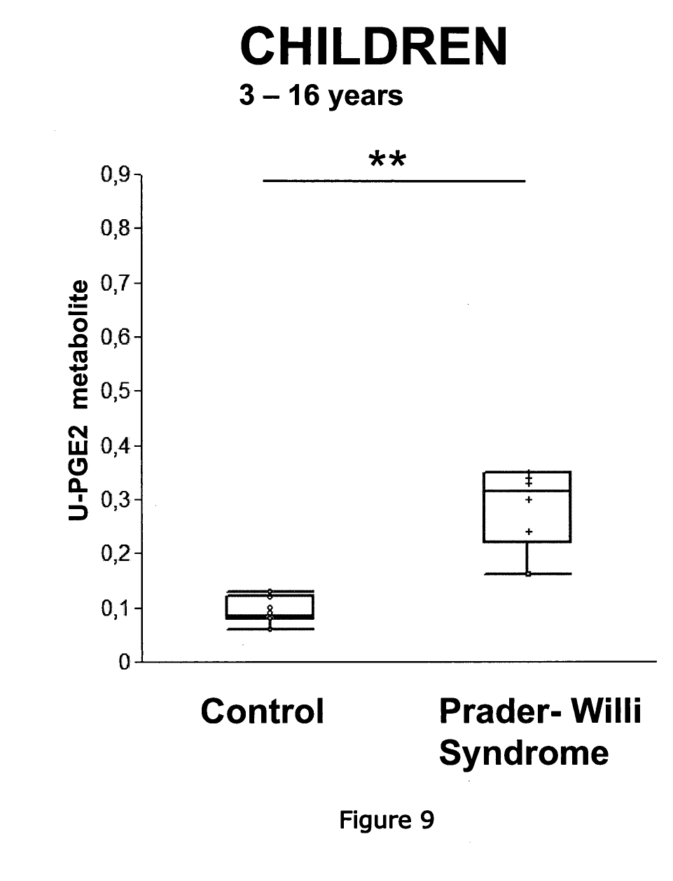

PWS children have a disturbed breathing pattern and autonomic control and are known to die suddenly (2-3% yearly prevalence) often in association with mild upper respiratory infection. As shown in FIG. 9, urinary PGEM levels in PWS children (n=6) were found to be significantly elevated in comparison with healthy control children. In FIG. 9 the y-axis shows urinary PGE metabolites in units of picomol PGEM/μg creatinine. The elevation of u-PGEM levels in this patient group (PWS) provides further evidence for the association between breathing disorders (particularly apnea), inflammation and PGE₂ (e.g. u-PGEM levels). It is presently believed that the presence of elevated prostaglandin metabolites in a sample (e.g. u-PGEM) obtained from a child (with or without PWS) may be indicative of increased likelihood of having or developing a breathing disorder, e.g. apnea, OSAS, SIDS and/or inflammation-related breathing disorder. Furthermore, a sub-population of children that have respiratory dysfunction that correlates with infection may, in particular, exhibit significant correlation between a breathing disorder and elevated prostaglandin metabolites in a sample (e.g. u-PGEM). This sub-population comprises children having: a) OSAS; and/or b) signs of autonomic dysfunction correlated with, for example PWS, Rett's syndrome or CCHS (Congenital hypoventilation syndrome, also known as “Ondine's curse”).

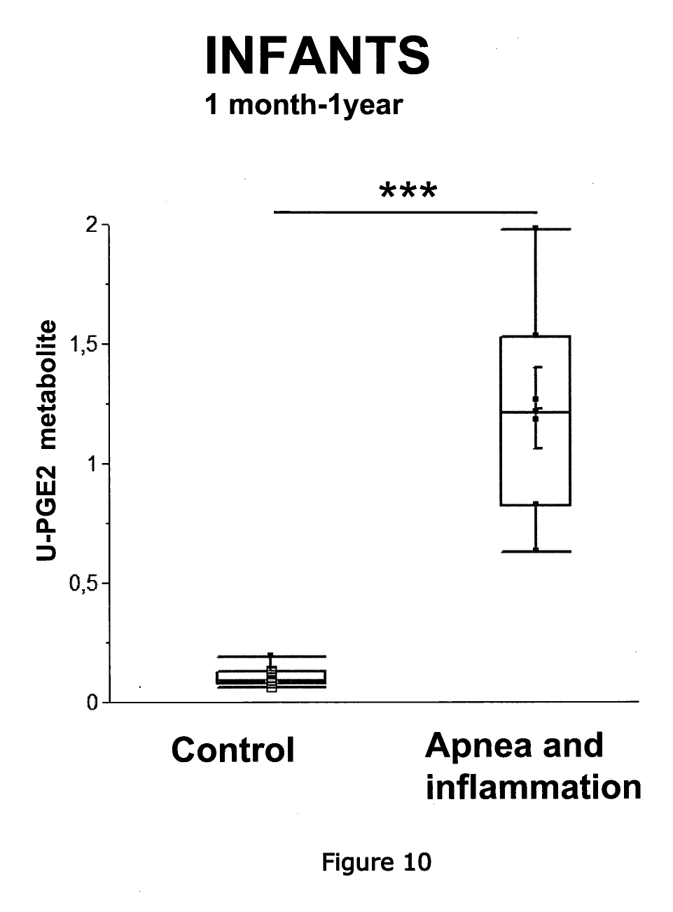

Furthermore, the present inventors have investigated u-PGEM levels in infants with ongoing inflammation (n=10) virus bronchiolitis and associated apnea. The results are shown in FIG. 10, in which the y-axis shows urinary PGE metabolites in units of picomol PGEM/μg creatinine. The infant group having ongoing inflammation and associated apnea displayed very high levels of u-PGEM compared with controls (n=10, infants and children without ongoing inflammation or apneas). Moreover, the CRP(C-reactive protein) levels, which are commonly used for assessment of infection in daily clinical care were only slightly elevated. Thus, measurement of u-PGEM levels may offer advantages in comparison with measuring CRP to evaluate ongoing inflammation, and also offers a potential mechanism for the dysregulated respiratory control seen in some young infants. Inflammation in sensitive children aged 1-6 months appears to be associated with irregular breathing and apneas primarily during sleep.

Viral infection (e.g. viral bronchioloitis) can cause severe breathing obstruction and central depression of the “breathing pacemaker” in the brainstem. However, such infection typically causes only a mild increase in CRP, a conventional marker for presence of an ongoing inflammatory disorder. Therefore, the measurement of prostaglandin metabolites (e.g. u-PGEM levels) is expected to provide indication of potential inflammation and/or breathing disorder at an earlier stage of the infection. Thus, an assay for levels of prostaglandin metabolites would be attractive in a clinical setting, and may enable a clinician to determine the severity of inflammation, prognosis and possible therapeutic intervention “at the bed”.

All references cited herein are incorporated herein by reference in their entirety and for all purposes to the same extent as if each individual publication or patent or patent application was specifically and individually indicated to be incorporated by reference in its entirety.

The specific embodiments described herein are offered by way of example, not by way of limitation. Any sub-titles herein are included for convenience only, and are not to be construed as limiting the disclosure in any way.

### REFERENCES

1. Fanaroff, A. A., et al. (1998) Pediatr Infect Dis 117, 593-598.
2. Prandota, J. (2004) Am J Ther 11, 517-546.
3. Guntheroth, W. (1989) Med Hypotheses 28, 121-123.
4. Dantzer, R. (2001) Ann N Y Acad Sci 933, 222-234.
5. Olsson, A., et al. (2003) Pediatr Res 54, 326-331.
6. Hofstetter, A. O. & Herlenius, E. (2005) Respir Physiol Neurobiol 146, 135-146.
7. Froen, J. F., et al. (2000) Pediatrics 105, E52.
8. Lindgren, C., Grogaard, 3 (1996) Acta Paediatr 85, 798-803.
9. Stoltenberg, L., et al. (1994) J Perinat Med 22, 421-432.
10. Vege, Å., et al. (1998) Acta Paediatr 87, 819-824.
11. Ericsson, A., et al. (1994) Journal of Neuroscience 14, 897-913.
12. Ericsson, A., et al. (1995) J Comp Neurol 361, 681-698.
13. Engblom, D., Ek, M, Saha, 5, Ericsson-Dahlstrand, A, Jakobsson, P J, Blomqvist, A (2002) J Mol Med 80, 5-15.
14. Coceani, F. & Akarsu, E. S. (1998) Ann N Y Acad Sci 856, 76-82.
15. Crestani, F., Seguy, F, Dantzer, R (1991) Brain Res 542, 330-335.
16. Ericsson, A., Arias, C, Sawchenko, P E (1997) J Neurosci 17, 7166-7179.
17. Guerra, F., Savich, R D, Wallen, L D, Lee, C H, Clyman, R I, Mauray, F E, Kitterman, J A (1988) Journal of Applied Physiology 64, 2160-2166.
18. Kitterman, J., Liggins, G C, Fewell, J E, Tooley, W H (1983) J Appl Physiol 54, 687-692.
19. Tai, T. C. & Adamson, S. L. (2000) Am J Physiol 278, 1460-1473.
20. Ek, M., Arias, C., Sawchenko, P., & Ericsson-Dahlstrand, A. (2000) J Comp Neurol 428, 5-20.
21. Nakamura, K., Kaneko, T., Yamashita, Y., Hasegawa, H., Katoh, H., & Negishi, M. (2000) J Comp Neuro/421, 543-569.
22. Gray, P. A., Rekling, J. C., Bocchiaro, C. M., & Feldman, J. L. (1999) Science 286, 1566-1568.
23. Ballanyi, K., Lalley, P. M., Hoch, B., & Richter, D. W. (1997) J Physiol 504, 127-134.
24. Pagliardini, S., Ren, J., & Greer, J. J. (2003) J Neurosci 23, 9575-9584.
25. Yamagata, K., Matsumura, K., Inoue, W., Shiraki, T., Suzuki, K., Yasuda, S., Sugiura, H., Cao, C., Watanabe, Y., & Kobayashi, S. (2001) J Neurosci 21, 2669-2677.
26. Kitterman, J., Liggins, G C, Clements, J A, Tooley, W H (1979) J Dev Physiol, 453-466.
27. Guerra, F. A., Savich, R. D., Clyman, R. I., & Kitterman, J. A. (1989) J Dev Physiol 11-6.
28. Long, W. (1988) J Appl Physiol 64, 409-418.
29. Guerra, F. A., Savich, R. D., Wallen, L. D., Lee, C. H., Clyman, R. I., Mauray, F. E., & Kitterman, J. A. (1988) J Appl Physiol 64, 2160-2166.
30. Lein, E. S., Hawrylycz, M. J., Ao, N., Ayres, M., Bensinger, A., Bernard, A., Boe, A. F., Boguski, M. S., Brockway, K. S., Byrnes, E. J., et al. (2007) Nature 445, 168-176.
31. Tai, T., MacLusky, N J, Adamson, S L (1994) Brain Res 652, 28-39.
32. Degi, R., Bari, F., Thore, C., Beasley, T., Thrikawala, N., & Busija, D. W. (1998) Neurobiology (Bp) 6, 467-468.
33. Shohami, E. & Gross, J. (1986) J Neurochem 47, 1678-1681.
34. Allen, L. G., Louis, T. M., & Kopelman, A. E. (1982) Biol Neonate 42, 8-14.
35. Ristimaki, A., Garfinkel, S., Wessendorf, J., Maciag, T., & Hla, T. (1994) Biol Chem 269, 11769-11775.
36. Degousee, N., Angoulvant, D., Fazel, S., Stefanski, E., Saha, S., Iliescu, K., Lindsay, T. F., Fish, J. E., Marsden, P. A., Li, R. K., et al. (2006) J Biol Chem 281, 16443-16452.
37. Savich, R. D., Guerra, F. A., Lee, C. C., & Kitterman, J. A. (1995) J Appl Physiol 78, 1477-1484.
38. Mural, D. T., Wallen, L. D., Lee, C. C., Clyman, R. I., Mauray, F., & Kitterman, J. A. (1987) J Appl Physiol 62, 271-277.
39. Feldman, J. L. & Del Negro, C. A. (2006) 7, 232-241.
40. McKay, L. C., Janczewski, W. A., & Feldman, J. L. (2005) Nature Neuroscience 8, 1142-1144.
41. Paton, J. F., Abdala, A. P., Koizumi, H., Smith, J. C., & St-John, W. M. (2006) Nat Neurosci 9, 311-313.
42. Tauman, R., Ivanenko, A., O'Brien, L. M., & Gozal, D. (2004) Pediatrics 113, e564-569.
43. Singh, G., Fong, L V, Salmon, A P, Keeton, B R (1994) Eur Heart J 15, 377-381.
44. Hoch, B. & Bernhard, M. (2000) Acta Paediatr 89, 1364-1368.
45. Hammerman, C. & Zangen, D. (1993) Crit Care Med 21, 154-155.
46. Schmidt, B., Davis, P., Moddemann, D., Ohlsson, A., Roberts, R. S., Saigal, S., Solimano, A., Vincer, M., & Wright, L. L. (2001) N Engl J Med 344, 1966-1972.
47. Trebino, C. E., Stock, J. L., Gibbons, C. P., Naiman, B. M., Wachtmann, T. S., Umland, J. P., Pandher, K., Lapointe, J. M., Saha, S., Roach, M. L., et al. (2003) Proc Natl Acad Sci USA 100, 9044-9049.
48. Fleming, E. F., Athirakul, K., Oliverio, M. I., Key, M., Goulet, J., Koller, B. H., & Coffman, T. M. (1998) Am J Physiol 275, F955-961.
49. Jacobi, M. S. & Thach, B. T. (1989) J Appl Physiol 66, 2384-2390.
50. Herlenius, E., Lagercrantz, H, Yamamoto, Y (1997) Pediatr Res 42, 46-53.
51. Suzue, T. (1984) J Physiol 354, 173-183.
52. Engblom, D., Saha, S., Engstrom, L., Westman, M., Audoly, L. P., Jakobsson, P. J., & Blomqvist, A. (2003) Nat Neurosci 6, 1137-1138.
53. Fewell, J. E., Smith, F. G., Ng, V. K., Wong, V. H., & Wang, Y. (2000) Am Physiol Regul Integr Comp Physiol 279, R39-46.
54. Hofstetter, A. O., et al. (2007) PNAS USA 104(23), 9894-9899.
55. Ward, E. S., et al. (1989) Nature 341, 544-546.
56. Bird et al. (1988) Science, 242, 423-426.
57. Huston et al. (1988) PNAS USA, 85, 5879-5883.
58. P. Holliger et al. (1993) PNAS USA, 90, 6444-6448.
59. Y. Reiter et al. (1996) Nature Biotech, 14, 1239-1245.
60. S. Hu et al. (1996) Cancer Res., 56, 3055-3061.
61. John et al. (2004) PLoS Biology, 11(2), 1862-1879.
62. Myers (2003) Nature Biotechnology, 21, 324-328.
63. Shinagawa et al. (2003) Genes and Dev., 17, 1340-1345.
64. Fire, A., et al. (1998) Nature 391, 806-811.
65. Fire, A., (1999) Trends Genet. 15, 358-363.
66. Sharp, P. A. (2001) Genes Dev. 15, 485-490.
67. Hammond, S. M., et al. (2001) Nature Rev. Genet. 2, 1110-1119.
68. Tuschl, T. (2001) Chem. Biochem. 2, 239-245.
69. Hamilton, A., et al. (1999) Science 286, 950-952.
70. Hammond, S. M., et al. (2000) Nature 404, 293-296.
71. Zamore, P. D., et al. (2000) Cell 101, 25-33.
72. Bernstein, E., et al. (2001) Nature 409 363-366.
73. Elbashir, S. M., et al. (2001) Genes Dev. 15, 188-200.
74. Elbashir, S. M., et al. (2001) Nature 411, 494-498.
75. Thoren et al. (2000) Eur. J. Biochem. 267, 6428.
76. Mancini et al. (2001) J. Biol. Chem. 276, 4469.
77. Quraishi et al. (2002) Biochem. Pharmacol. 63, 1183.
78. Schultz, J. S., (1996) Biotechnol. Prog. 12, 729-743.
79. Cutz, E. et al. (1997) Am. J. Respir. Crit. Care Med. 155(1), 358-363.
80. Festen, D. A. et al. (2006) J. Clin. Endocrinol. Metab. 91(12), 4911-4915.
81. Debra, E. et al. (2007) Am. J. Med. Gen. Part A, 771-788.
82. Paterson, D. S. et al. (2006) JAMA 296(17), 2124-2132.
83. Onodera, H. et al. (2000) Lancet 356(9231), 739-740.
84. Yang, 3. et al. (1994) Biochem. Biophys. Res. Commun. 198(3), 999-1006.
85. Herlenius, E. and Lagercrantz, H. (1999), J. Physiol. 518 (Pt 1), 159-172.
86. Ballanyi, K. et al. (1997) J. Physiol. 504(1), 127-134.
87. Katoh, H. et al. (1996) Journal of Biological Chemistry 271(47), 29780-29784.
88. van Rodijnen, W. F. et al. (2007) Am. J. Physiol. Renal Physiol. 292(3), F1094-1101.
89. Rowland, S. E. et al. (2007) Eur. J. Pharmacol. 560(2-3), 216-224.
90. Habib, A. et al. (2007) Faseb. J. 21(8), 1665-1674.
91. Yao, J. C. et al. (2007) Yakugaku Zasshi 127(3), 527-532.
92. Morrell, M. J., and Twigg, G. (2006) Adv. Exp. Med. Biol. 588, 75-88.
93. Rigatto, H., (2003) “Periodic Breathing”. In: Mathew O P, editor. Respiratory control and disorders in the newborn, Vol 173. New York: Marcel Dekker, Inc; 237-72.
94. Simmons, D. L. et al. (2004) Pharmacol Rev. 56(3), 387-437.
95. Dubowitz, L. and Dubowitz, V. (1981) Clinical Developmental Medicine 79, London, England: Mac Keith Press.
96. Sarnat, H. B. and Sarnat, M. S. (1976) Arch Neurol. 33, 696-705.
97. Volpe, J. J. (1994) Hypoxic-ischaemic encephalopathy: Neuropathology and pathogenesis In Neurology of the Newborn, pp. 279-314. Philadelphia: WB Saunders Co.
98. Roth, S. C., et al. (1992) Dev Med and Child Neurol. 34:285-295.
99. Hanrahan, D., et al. (1999) Dev Med and Child Neurol. 41:76-82.
100. Mehmet, H., et al. (1994) Neurosci Lett. 181:121-125.
101. Greher, J. K. and Nelson, K. B. (1997). J. Am. Med. Association. 278:207-211.
102. Nelson, K. B., et al. (1998) Ann Neurol. 44(4):665-75.
103. Foster-Barber, A., and Ferriero, D. M. (2002) Mental retard and dev disabilities. 8:20-24.
104. Silveira, R. C., and Procianoy, R. S. (2003). J. Pediatr. 625-629.
105. Deems et al. (2007) Methods Enzymol. 432, 59-82.
106. Welsh, T. N. et al. (2007) Prostaglandins & other Lipid Mediators 83, 304-310.
107. Tauber, M. et al. (2008) Am J Med Genet A. 146(7):881-887.
108. Perez, I. A. and Ward, S. L. (2008) Pediatr Ann. 37(7):465-470.
109. Wildhaber, J. H. and Moeller, A. (2007) Swiss Med. Wkly. 137(49-50):689-694.
110. Tarasiuk, A. (2007) Am J Respir Crit Care Med. 175(1):55-61.
111. Weber, M. A. (2008) Lancet 371:1848-1853.
112. Hoch, B. et al. (2000) Prostaglandins Other Lipid Mediat. 60(1-3):9-14.
113. Hoch, B. and Bernhard, M. (2000) Acta Paediatr. 89(11):1364-1368.
114. Hofstetter, A. O. et al. (2007) PNAS 104(23):9894-9899.
115. Festen, D. A. et al. (2006) Clin Endocrinol Metab. 91(12):4911-4915.

### SEQUENCES REFERRED TO HEREIN

```
SEQ ID NO: 1
      1 gatcgtgtag gccggccgca ccatgggggg cagcccagcc cagccgcggt aaacgccgac
    61 ctccgccgcc gcccgcgccg cgtctgcccc ctccgctgcg gctctctgga cgccatcccc
  121 tcctcacctc gaagccaaca tgaaggagac ccggggctac ggaggggatg cccccttctg
  181 cacccgcctc aaccactcct acacaggcat gtgggcgccc gacggttccg ccgaggcgcg
  241 gggcaacctc acgcgccctc cagggtctgg cgaggattgc ggatcggtgt ccgtggcctt
  301 cccgatcacc atgctgctca ctggtttcgt gggcaacgca ctggccatgc tgcttgtgtc
  361 gcgcagctac cggcgccggg agagcaagcg caagaagtcc ttcctgctgt gcatcggctg
  421 gctggcgctc accgacctgg tcgggcagct tctcaccacc ccggtcgtca tcgtcgtgta
  481 cctgtccaag cagcgttggg agcacatcga cccgtcgggg cggctctgca cctttttcgg
  541 gctgaccatg actgttttcg ggctctcctc gttgttcatc gccagcgcca tggcggtcga
  601 gcgggcgctg gccatcaggg cgccgcactg gtatgcgagc cacatgaaga cgcgtgccac
  661 ccgcgctgtg ctgctcggcg tgtggctggc cgtgctcgcc ttcgccctgc tgccggtgct
  721 gggcgtgggc cagtacaccg tccagtggcc cgggacgtgg tgcttcatca gcaccgggcg
  781 agggggcaac gggactagct cttcgcataa ctggggcaac cttttcttcg cctctgcctt
  841 tgccttcctg gggctcttgg cgctgacagt caccttttcc tgcaacctgg ccaccattaa
  901 ggccctggtg tcccgctgcc gggccaaggc cacggcatct cagtccagtg cccagtgggg
  961 ccgcatcacg accgagacgg ccattcagct tatggggatc atgtgcgtgc tgtcggtctg
1021 ctggtctccg ctcctgataa tgatgttgaa aatgatcttc aatcagacat cagttgagca
1081 ctgcaagaca cacacggaga agcagaaaga atgcaacttC ttcttaatag ctgttcgcct
1141 ggcttcactg aaccagatct tggatccttg ggtttacctg ctgttaagaa agatccttct
1201 tcgaaagttt tgccagatca ggtaccacac aaacaactat gcatccagct ccacctcctt
1261 accctgccag tgttcctcaa ccttgatgtg gagcgaccat ttggaaagat aatgaaagaa
1321 cggagttgga cattttattg caattcctgc ttccctgaat ttgcatattt cttcccacct
1381 gagaaggata attatatatt ttaatttgga ttatttcttc attttatctt ttatttaatg
1441 attgttttgt cagtaatacc catggagatc aaatttatta ttataatcca tgcctctgaa
1501 tattagattg gtttc
SEQ ID NO: 2
MKETRGYGGDAPFCTRLNHSYTGMWAPDGSAEARGNLTRPPGSGEDCGSVSVAFPITMLLTGFVGNALAM
LLVSRSYRRRESKRKKSFLLCIGWLALTDLVGQLLTTPVVIVVYLSKQRWEHIDPSGRLCTFFGLTMTVF
GLSSLFIASAMAVERALAIRAPHWYASHMKTRATRAVLLGVWLAVLAFALLPVLGVGQYTVQWPGTWCFI
STGRGGNGTSSSHNWGNLFFASAFAFLGLLALTVTFSCNLATIKALVSRCRAKATASQSSAQWGRITTET
AIQLMGIMCVLSVCWSPLLIMMLKMIFNQTSVEHCKTHTEKQKECNFFLIAVRLASLNQILDPWVYLLLR
KILLRKFCQTRYHTNNYASSSTSLPCQCSSTLMWSDHLER
SEQ ID NO: 3
atgcctgccc acagcctggt gatgagcagc ccggccctcc cggccttcct gctctgcagc 60
acgctgctgg tcatcaagat gtacgtggtg gccatcatca cgggccaagt gaggctgcgg 120
aagaaggcct ttgccaaccc cgaggatgcc ctgagacacg gaggccccca gtattgcagg 180
agtgaccccg acgtggaacg ctgcctcagg gcccaccgga acgacatgga gaccatctac 240
cccttccttt tcctgggctt cgtctactcc tttctgggtc ctaacccttt tgtcgcctgg 300
atgcacttcc tggtcttcct cgtgggccgt gtggcacaca ccgtggccta cctggggaag 360
ctgcgggcac ccatccgctc cgtgacctac accctggccc agctcccctg cgcctccatg 420
gctctgcaga tcctctggga agcggcccgc cacctgtga 459
SEQ ID NO: 4
Met Pro Ala His Ser Leu Val Met Ser Ser Pro Ala Leu Pro Ala Phe
    1 5 10 15
Leu Leu Cys Ser Thr Leu Leu Val Ile Lys Met Tyr Val Val Ala Ile
                          20 25 30
Ile Thr Gly Gln Val Arg Leu Arg Lys Lys Ala Phe Ala Asn Pro Glu
                  35 40 45
Asp Ala Leu Arg His Gly Gly Pro Gln Tyr Cys Arg Ser Asp Pro Asp
          50 55 60
Val Glu Arg Cys Leu Arg Ala His Arg Asn Asp Met Glu Thr Ile Tyr
  65 70 75 80
Pro Phe Leu Phe Leu Gly Phe Val Tyr Ser Phe Leu Gly Pro Asn Pro
                                  85 90 95
Phe Val Ala Trp Met His Phe Leu Val Phe Leu Val Gly Arg Val Ala
                        100 105 110
His Thr Val Ala Tyr Leu Gly Lys Leu Arg Ala Pro Ile Arg Ser Val
                115 120 125
Thr Tyr Thr Leu Ala Gln Leu Pro Cys Ala Ser Met Ala Leu Gln Ile
          130 135 140
Leu Trp Glu Ala Ala Arg His Leu
145 150
SEQ ID NO: 5
      1 caattgtcat acgacttgca gtgagcgtca ggagcacgtc caggaactcc tcagcagcgc
    61 ctccttcagc tccacagcca gacgccctca gacagcaaag cctacccccg cgccgcgccc
  121 tgcccgccgc tcggatgctc gcccgcgccc tgctgctgtg cgcggtcctg gcgctcagcc
  181 atacagcaaa tccttgctgt tcccacccat gtcaaaaccg aggtgtatgt atgagtgtgg
  241 gatttgacca gtataagtgc gattgtaccc ggacaggatt ctatggagaa aactgctcaa
  301 caccggaatt tttgacaaga ataaaattat ttctgaaacc cactccaaac acagtgcact
  361 acatacttac ccacttcaag ggattttgga acgttgtgaa taacattccc ttccttcgaa
  421 atgcaattat gagttatgtc ttgacatcca gatcacattt gattgacagt ccaccaactt
  481 acaatgctga ctatggctac aaaagctggg aagccttctc taacctctcc tattatacta
  541 gagcccttcc tcctgtgcct gatgattgcc cgactccctt gggtgtcaaa ggtaaaaagc
  601 agcttcctga ttcaaatgag attgtggaaa aattgcttct aagaagaaag ttcatccctg
  661 atccccaggg ctcaaacatg atgtttgcat tctttgccca gcacttcacg catcagtttt
  721 tcaagacaga tcataagcga gggccagctt tcaccaacgg gctgggccat ggggtggact
  781 taaatcatat ttacggtgaa actctggcta gacagcgtaa actgcgcctt ttcaaggatg
  841 gaaaaatgaa atatcagata attgatggag agatgtatcc tcccacagtc aaagatactc
  901 aggcagagat gatctaccct cctcaagtcc ctgagcatct acggtttgct gtggggcagg
  961 aggtctttgg tctggtgcct ggtctgatga tgtatgccac aatctggctg cgggaacaca
1021 acagagtatg cgatgtgctt aaacaggagc atcctgaatg gggtgatgag cagttgttcc
1081 agacaagcag gctaatactg ataggagaga ctattaagat tgtgattgaa gattatgtgc
1141 aacacttgag tggctatcac ttcaaactga aatttgaccc agaactactt ttcaacaaac
1201 aattccagta ccaaaatcgt attgctgctg aatttaacac cctctatcac tggcatcccc
1261 ttctgcctga cacctttcaa attcatgacc agaaatacaa ctatcaacag tttatctaca
1321 acaactctat attgctggaa catggaatta cccagtttgt tgaatcattc accaggcaaa
1381 ttgctggcag ggttgctggt ggtaggaatg ttccacccgc agtacagaaa gtatcacagg
1441 cttccattga ccagagcagg cagatgaaat accagtcttt taatgagtac cgcaaacgct
1501 ttatgctgaa gccctatgaa tcatttgaag aacttacagg agaaaaggaa atgtctgcag
1561 agttggaagc actctatggt gacatcgatg ctgtggagct gtatcctgcc cttctggtag
1621 aaaagcctcg gccagatgcc atctttggtg aaaccatggt agaagttgga gcaccattct
1681 ccttgaaagg acttatgggt aatgttatat gttctcctgc ctactggaag ccaagcactt
1741 ttggtggaga agtgggtttt caaatcatca acactgcctc aattcagtct ctcatctgca
1801 ataacgtgaa gggctgtccc tttacttcat tcagtgttcc agatccagag ctcattaaaa
1861 cagtcaccat caatgcaagt tcttcccgct ccggactaga tgatatcaat cccacagtac
1921 tactaaaaga acgttcgact gaactgtaga agtctaatga tcatatttat ttatttatat
1981 gaaccatgtc tattaattta attatttaat aatatttata ttaaactcct tatgttactt
2041 aacatcttct gtaacagaag tcagtactcc tgttgcggag aaaggagtca tacttgtgaa
2101 gacttttatg tcactactct aaagattttg ctgttgctgt taagtttgga aaacagtttt
2161 tattctgttt tataaaccag agagaaatga gttttgacgt ctttttactt gaatttcaac
2221 ttatattata agaacgaaag taaagatgtt tgaatactta aacactatca caagatggca
2281 aaatgctgaa agtttttaca ctgtcgatgt ttccaatgca tcttccatga tgcattagaa
2341 gtaactaatg tttgaaattt taaagtactt ttggttattt ttctgtcatc aaacaaaaac
2401 aggtatcagt gcattattaa atgaatattt aaattagaca ttaccagtaa tttcatgtct
2461 actttttaaa atcagcaatg aaacaataat ttgaaatttc taaattcata gggtagaatc
2521 acctgtaaaa gcttgtttga tttcttaaag ttattaaact tgtacatata ccaaaaagaa
2581 gctgtcttgg atttaaatct gtaaaatcag atgaaatttt actacaattg cttgttaaaa
2641 tattttataa gtgatgttcc tttttcacca agagtataaa cctttttagt gtgactgtta
2701 aaacttcctt ttaaatcaaa atgccaaatt tattaaggtg gtggagccac tgcagtgtta
2761 tctcaaaata agaatatttt gttgagatat tccagaattt gtttatatgg ctggtaacat
2821 gtaaaatcta tatcagcaaa agggtctacc tttaaaataa gcaataacaa agaagaaaac
2881 caaattattg ttcaaattta ggtttaaact tttgaagcaa actttttttt atccttgtgc
2941 actgcaggcc tggtactcag attttgctat gaggttaatg aagtaccaag ctgtgcttga
3001 ataacgatat gttttctcag attttctgtt gtacagttta atttagcagt ccatatcaca
3061 ttgcaaaagt agcaatgacc tcataaaata cctcttcaaa atgcttaaat tcatttcaca
3121 cattaatttt atctcagtct tgaagccaat tcagtaggtg cattggaatc aagcctggct
3181 acctgcatgc tgttcctttt cttttcttct tttagccatt ttgctaagag acacagtctt
3241 ctcatcactt cgtttctcct attttgtttt actagtttta agatcagagt tcactttctt
3301 tggactctgc ctatattttc ttacctgaac ttttgcaagt tttcaggtaa acctcagctc
3361 aggactgcta tttagctcct cttaagaaga ttaaaagaga aaaaaaaagg cccttttaaa
3421 aatagtatac acttatttta agtgaaaagc agagaatttt atttatagct aattttagct
3481 atctgtaacc aagatggatg caaagaggct agtgcctcag agagaactgt acggggtttg
3541 tgactggaaa aagttacgtt cccattctaa ttaatgccct ttcttattta aaaacaaaac
3601 caaatgatat ctaagtagtt ctcagcaata ataataatga cgataatact tcttttccac
3661 atctcattgt cactgacatt taatggtact gtatattact taatttattg aagattatta
3721 tttatgtctt attaggacac tatggttata aactgtgttt aagcctacaa tcattgattt
3781 ttttttgtta tgtcacaatc agtatatttt ctttggggtt acctctctga atattatgta
3841 aacaatccaa agaaatgatt gtattaagat ttgtgaataa atttttagaa atctgattgg
3901 catattgaga tatttaaggt tgaatgtttg tccttaggat aggcctatgt gctagcccac
3961 aaagaatatt gtctcattag cctgaatgtg ccataagact gaccttttaa aatgttttga
4021 gggatctgtg gatgcttcgt taatttgttc agccacaatt tattgagaaa atattctgtg
4081 tcaagcactg tgggttttaa tatttttaaa tcaaacgctg attacagata atagtattta
4141 tataaataat tgaaaaaaat tttcttttgg gaagagggag aaaatgaaat aaatatcatt
4201 aaagataact caggagaatc ttctttacaa ttttacgttt agaatgttta aggttaagaa
4261 agaaatagtc aatatgcttg tataaaacac tgttcactgt tttttttaaa aaaaaaactt
4321 gatttgttat taacattgat ctgctgacaa aacctgggaa tttgggttgt gtatgcgaat
4381 gtttcagtgc ctcagacaaa tgtgtattta acttatgtaa aagataagtc tggaaataaa
4441 tgtctgttta tttttgtact attta
SEQ ID NO: 6
```

```
    1 MLARALLLCA VLALSHTANP CCSHPCQNRG VCMSVGFDQY KCDCTRTGFY GENCSTPEFL
  61 TRIKLFLKPT PNTVHYILTH FKGFWNVVNN IPFLRNAIMS YVLTSRSHLI DSPPTYNADY
121 GYKSWEAFSN LSYYTRALPP VPDDCPTPLG VKGKKQLPDS NEIVEKLLLR RKFIPDPQGS
181 NMMFAFFAQH FTHQFFKTDH KRGPAFTNGL GHGVDLNHIY GETLARQRKL RLFKDGKMKY
241 QIIDGEMYPP TVKDTQAEMI YPPQVPEHLR FAVGQEVFGL VPGLMMYATI WLREHNRVCD
301 VLKQEHPEWG DEQLFQTSRL ILIGETIKIV IEDYVQHLSG YHFKLKFDPE LLFNKQFQYQ
361 NRIAAEFNTL YHWHPLLPDT FQIHDQKYNY QQFIYNNSIL LEHGITQFVE SFTRQIAGRV
421 AGGRNVPPAV QKVSQASIDQ SRQMKYQSFN EYRKRFMLKP YESFEELTGE KEMSAELEAL
481 YGDIDAVELY PALLVEKPRP DAIFGETMVE VGAPFSLKGL MGNVICSPAY WKPSTFGGEV
541 GFQIINTASI QSLICNNVKG CPFTSFSVPD PELIKTVTIN ASSSRSGLDD INPTVLLKER
601 STEL
```

1. A method of treating a breathing disorder in a mammalian subject, comprising administering to the subject a composition comprising: an inhibitor of E-prostanoid receptor subtype 3 (EP3R); an inhibitor of microsomal prostaglandin E synthase-1 (mPGES-1); and/or a selective inhibitor of cyclooxygenase-2 (COX-2).

2. A method according to claim 1, wherein the composition comprises an inhibitor of EP3R.

3. A method according to claim 2, wherein the inhibitor of EP3R is a specific binding member that binds an EP3R polypeptide or a nucleic acid that down regulates expression of an EP3R-encoding gene.

4. A method according to claim 2, wherein the inhibitor of EP3R is (2E)-N-[(5-bromo-2-methoxyphenyl)sulfonyl]-3-[5-chloro-2-(2-naphthylmethyl)phenyl]-acrylamide (L826266) or a pharmaceutically acceptable salt thereof.

5. A method according to claim 1, wherein the composition comprises an inhibitor of mPGES-1.

6. A method according to claim 5, wherein the inhibitor of mPGES-1 is a specific binding member that binds an mPGES-1 polypeptide or a nucleic acid that down regulates expression of an mPGES-1-encoding gene.

7. A method according to claim 5, wherein the inhibitor of mPGES-1 is 3-[tert-Butylthio-1-(4-chlorobenzyl)-5-isopropyl-1H-indol-2-yl]-2,2-dimethylpropionic acid (MK-886) or a pharmaceutically acceptable salt thereof.

8. A method according to claim 1, wherein the composition comprises a selective inhibitor of COX-2.

9. A method according to claim 8, wherein the selective inhibitor of COX-2 is a specific binding member that binds a COX-2 polypeptide or a nucleic acid that down regulates expression of a COX-2-encoding gene.

10. A method according to claim 8, wherein the selective inhibitor of COX-2 is: 4-(5-methyl-3-phenylisoxazol-4-yl)benzenesulfonamide (valdecoxib) or a pharmaceutically acceptable salt thereof; 4-[5-(4-methylphenyl)-3-(trifluoromethyl)pyrazol-1-yl]benzenesulfonamide (celecoxib) or a pharmaceutically acceptable salt thereof; or 4-(4-methylsulfonylphenyl)-3-phenyl-5H-furan-2-one (rofecoxib) or a pharmaceutically acceptable salt thereof.

11. A method of assessing susceptibility to, or presence of, a breathing disorder in a mammalian subject, comprising
detecting the level of prostaglandin-E₂ (PGE₂), or a metabolite thereof, in a sample from the subject, and
comparing the level in the sample with a control level of PGE₂, or the metabolite thereof,
wherein an elevated level of PGE₂, or the metabolite thereof, in the sample compared with the control level of PGE₂, or the metabolite thereof, indicates susceptibility to, or presence of, a breathing disorder in the subject.

12. A method according to claim 11, wherein the sample comprises a urine sample or a cerebrospinal fluid (CSF) sample.

13. A method according to claim 11, further comprising
detecting the level of C-reactive protein (CRP) in a sample from the subject, and
comparing the level in the sample with a control level of CRP,
wherein an elevated level of CRP in the sample compared with the control level of CRP indicates susceptibility to, or presence of, a breathing disorder in the subject.

14. A method according to claim 11, wherein the breathing disorder is apnea, periodic breathing or failure to autoresuscitate following a hypoxic event.

15. A method according to claim 11, wherein the breathing disorder is a breathing disorder that occurs during sleep, particularly obstructive sleep apnea syndrome.

16. A method according to claim 11, wherein the breathing disorder is an infection-associated breathing disorder.

17. A method according to claim 16, wherein the infection-associated breathing disorder is an IL-1β-related breathing disorder.

18. A method according to claim 14, wherein the breathing disorder is apnea following a hypoxic event.

19. A method according to claim 15, wherein the apnea is induced by the hypoxic event.

20. A method according to claim 11, wherein the mammalian subject is a human subject.

21. A method according to claim 20, wherein the human subject is less than 5 years of age.

22. A method according to claim 21, wherein the breathing disorder is a disorder that results in, or increases the likelihood of, sudden infant death syndrome (SIDS).

23. A method according to claim 18, wherein the hypoxic event is perinatal asphyxia.

24. A method according to claim 20, wherein the human subject is greater than 18 years of age.

25. A method according to claim 24, wherein the breathing disorder is adult sleep apnea.

26. A method of assessing susceptibility to, or presence of, hypoxic ischemic encephalopathy (HIE) in a mammalian subject, comprising
detecting the level of prostaglandin-E₂ (PGE₂), or a metabolite thereof, in a sample from the subject, and
comparing the level in the sample with a control level of PGE₂, or the metabolite thereof,
wherein an elevated level of PGE₂, or the metabolite thereof, in the sample compared with the control level of PGE₂ indicates susceptibility to, or presence of, HIE in the subject.

27. A method according to claim 26, comprising grading the severity of HIE in the subject by measuring the degree of elevation of the level of PGE₂, or the metabolite thereof, in the sample compared with the control level of PGE₂, or the metabolite thereof.

28. A method of assessing perinatal asphyxia to which a mammalian subject has been subjected, comprising
detecting the level of prostaglandin-E₂ (PGE₂), or a metabolite thereof, in a sample from the subject, and
comparing the level in the sample with a control level of PGE₂, or the metabolite thereof,
wherein an elevated level of PGE₂, or the metabolite thereof, in the sample compared with the control level of PGE₂ indicates that the subject has been subjected to perinatal asphyxia.

29. A method according to claim 28, comprising grading the severity of the perinatal asphyxia to which the subject has been subjected by measuring the degree of elevation of the level of PGE₂, or the metabolite thereof, in the sample compared with the control level of PGE₂, or the metabolite thereof.

30. A method according to claim 26, wherein the sample is a cerebrospinal fluid (CSF), urine or blood sample taken within 7 days of birth of the subject.

31. A method according to claim 30, wherein the sample is taken within 24 hours of birth of the subject.

32. A method according to claim 26, wherein the mammalian subject is a human subject.

33. A method according to claim 32, further comprising measuring the Apgar score of the human subject within 30 minutes of birth.

34. A method according to claim 33, wherein the Apgar score is measured at about 1, 5, 10, 15 and/or 20 minutes after birth.

35. A method for identifying a substance for use in treating a breathing disorder in a mammal, comprising assaying a test substance for the ability to inhibit one or more of the following:
(a) COX-2-mediated synthesis of PGH₂;
(b) mPGES-1-mediated conversion of a cyclic endoperoxide substrate of mPGES-1 into a product which is the 9-keto, 11α hydroxy form of the substrate; and
(c) EP3R agonist-mediated activation of EP3R,
wherein inhibition of one or more of (a), (b) and (c) indicates that the test substance is a substance for use in treating a breathing disorder in a mammal.

36. A method according to claim 35, comprising:
contacting a COX-2 polypeptide with a test substance and arachidonic acid, under conditions in which arachidonic acid would be converted to PGH₂ by COX-2 in the absence of the test substance; and
determining the level of PGH₂ production in the presence of the test substance compared with a control level of PGH₂ production in the absence of the test substance,
wherein a lower level of PGH₂ production in the presence of the test substance compared with said control level indicates that the test substance is an agent for use in treating a breathing disorder in a mammal.

37. A method according to claim 36, comprising detecting a lower level of PGH₂ production in the presence of the test substance compared with the control level, and thereby identifying the test substance as a substance for use in treating a breathing disorder in a mammal.

38. A method according to claim 35, comprising:
contacting an mPGES-1 polypeptide with a test substance and a cyclic endoperoxide substrate of mPGES-1, under conditions in which the cyclic endoperoxide substrate of mPGES-1 would be converted by mPGES-1 into a product which is the 9-keto, 11α hydroxy form of the substrate in the absence of the test substance; and
determining the level of production of the product in the presence of the test substance compared with a control level of production of the product in the absence of the test substance,
wherein a lower level of production of the product in the presence of the test substance compared with said control level indicates that the test substance is a substance for use in treating a breathing disorder in a mammal.

39. A method according to claim 38, comprising detecting a lower level of production of the product in the presence of the test substance compared with the control level, and thereby identifying the test substance as a substance for use in treating a breathing disorder in a mammal.

40. A method according to claim 35, comprising:
contacting an EP3R polypeptide with a test substance and an EP3R agonist under conditions in which the EP3R agonist would activate the EP3R polypeptide in the absence of the test substance; and
determining the level of EP3R polypeptide activation in the presence of the test substance compared with a control level of EP3R polypeptide activation in the absence of the test substance,
wherein a lower level of EP3R polypeptide activation in the presence of the test substance compared with said control level indicates that the test substance is a substance for use in treating a breathing disorder in a mammal.

41. A method according to claim 40, comprising detecting a lower level of EP3R polypeptide activation in the presence of the test substance compared with the control level, and thereby identifying the test substance as a substance for use in treating a breathing disorder in a mammal.

42. A method for identifying a substance for use in treating a breathing disorder in a mammal, comprising:
administering a test substance to a test mammal, wherein the test substance is an inhibitor of EP3R, an inhibitor of mPGES-1 and/or a selective inhibitor of COX-2; and
determining the severity of a sign or symptom of a breathing disorder in the test mammal compared to the sign or symptom in a control mammal to which the test substance has not been administered,
wherein a lower severity of the sign or symptom of the breathing disorder in the test mammal than in the control mammal indicates that the test substance is a substance for use in treating a breathing disorder in a mammal.

43. A method according to claim 42, wherein the sign or symptom is selected from: respiratory depression, decreased breathing frequency, decreased tidal volume and decreased gasping in response to hypoxia.

44. A method according to claim 42, comprising administering IL-1β or LPS before determining the severity of the sign or symptom.

45. A method according to claim 35, wherein the test substance is identified as a substance for use in treating a breathing disorder in a mammal, and wherein the method further comprises formulating the test substance into a composition comprising a pharmaceutically acceptable excipient.

46. A method of assessing the presence of and/or severity of hypoxia and/or apnea in a human subject, comprising
detecting the level of one or more PGE₂ metabolites in a urine sample obtained from the subject, and
comparing the level in the sample with a control level of said one or more PGE₂ metabolites,
wherein a level of said one or more PGE₂ metabolites that is at least 20%, at least 50%, at least 100% or at least 200% greater in the sample compared with the control level of said one or more PGE2 metabolites indicates the presence of and/or greater severity of hypoxia and/or apnea in the subject.

47. A method according to claim 46, wherein the human subject has obstructive sleep apnea syndrome (OSAS), an autonomic dysfunction disorder, such as Prader-Willi Syndrome, Congenital Hypoventilation Syndrome or Rett's Syndrome.

48. A method according to claim 46, wherein the human subject is greater than 16 years of age.

49. A method according to claim 46, wherein the human subject is between 1 and 16 years of age.

50. A method according to claim 46, wherein the human subject is between 0 and 1 year of age.
